SEQUENCE LISTING (86 sequences)

SEQ ID NO: 1 - DNA, 573 bp, Bacillus subtilis
atgaaagaagttaataaagagcaaatcgaacaagctgttcgtcaaattttagaagcgatcggagaagacccgaatagagaagggcttcttgatactccgaaaagagtcgcaaagatgtatgccgaagtattctccggcttgaatgaagatccaaaagaacatttccagactatcttcggtgaaaaccatgaggagcttgttcttgtaaaagatatagcgtttcattctatgtgtgagcatcaccttgttcccttttatggaaaagcacatgttgcatatatcccgcgaggcggaaaggtcacaggactcagcaaactggcacgtgccgttgaagccgttgcaaagcgcccgcagcttcaggaacgcatcacttctacaattgcagaaagcatcgtagaaacgcttgatccgcatggcgtaatggtagtggttgaagcggaacacatgtgcatgacgatgcgcggtgtaagaaaaccgggtgcgaaaactgtgacttcagcagtcagaggcgtttttaaagatgatgccgctgcccgtgcagaagtattggaacatattaaacgccaggactaa

SEQ ID NO: 2 - DNA, 363 bp, Bacillus subtilis
atggataaagtttatgtagaaggtatggagttttacggatatcacggtgtgttcacagaagaaaacaaacttggccagcggtttaaagtcgatttaaccgctgagctggatttaagcaaagctggacagacagacgaccttgagcaaacgatcaactatgctgagctctatcacgtatgtaaagatatcgtggaaggggagcctgtgaaattggtggaaacgctggcggaacgtattgctggcactgttctcggaaaatttcagcctgttcagcaatgtacggtgaaagtgattaagccagacccgccaattcccggacactataaatcagtagcaattgaaattacgagaaaaaagtcatga

SEQ ID NO: 3 - DNA, 504 bp, Bacillus subtilis
atgaacaacatagcttatattgcacttggatctaatattggagatagagaaacgtatttaaggcaagcagtggctttactgcatcagcatgctgcggtgacagtcactaaagtgtcgtctatttacgaaactgacccggtcggatacgaagatcaagctcaatttttgaatatggctgttgaaatcaagacatcattgaacccttttgaactccttgaactgacgcagcagatagaaaatgaattaggcagaacaagggaagtaagatgggggccgcggacggcagaccttgacattttgttatttaatcgtgaaaatattgaaacagagcaactaattgttccgcatccgagaatgtatgagcgtttgtttgtccttgcgccgcttgcggaaatttgccagcaggttgaaaaagaggctacaagcgccgaaacagaccaagaaggtgtaagagtatggaagcagaaatctggggtagacgaattcgtgcattcagaaagctga

SEQ ID NO: 4 - DNA, 858 bp, Bacillus subtilis
atggcgcagcacacaatagatcaaacacaagtaatccacactaagcccagcgctttatcatataaagagaagacgctggtgatgggaattttaaacgtaacgcctgactctttctcggacggcggaaaatatgacagcttggacaaggcgctgctgcacgcgaaagagatgatcgatgatggtgcccatatcattgatattggaggggaatcgacaaggcctggcgctgagtgcgtatctgaggatgaggagatgtccagagtcattccggtgattgagcggattacgaaagagcttggtgttcctatttctgtagacacgtacaaggcttctgtcgcagatgaagcagtgaaagccggtgcatccattatcaatgatatttggggagccaaacatgatccgaagatggcttccgttgcagctgaacataatgttccaattgtactcatgcataaccgccctgaaagaaactacaatgacttattgccggatatgctgtcggacttaatggagagtgtaaaaattgctgttgaggccggagtagacgagaagaacattattcttgatcctggtatcggtttcgcgaaaacctatcacgataacttggcagtgatgaacaaactagagattttcagcggattgggatatccggttcttctggcaacctcccgaaaaagattcatcggacgtgttctggatcttccgcctgaggagcgggctgagggcacaggcgcgactgtgtgtctcggcattcaaaaaggctgtgacattgtcagggtccatgatgtaaagcaaattgccagaatggcgaaaatgatggacgcgatgctgaataagggaggggtgcaccatggataa

SEQ ID NO: 5 - DNA, 1293 bp, Bacillus subtilis
ttatttcaacctttttcgaatgtcagaaataaagtaaagagatccggtaatcagcacaatttcatttgagccctttttactttctatgaatttgattacatcgtctggatcttcactccagcttttattgctgatttcacttgcatcatagagatcttttgcaagggaagcacgcgggaaatcaaaagaagcaaaatgaatcgcatgagcaatggtttccagtcttttaatcatgttctgatacggtttgtcctttaacgcgctaaacaccacagaaatgcgtgaattggcgaaacgctgcttcatcgtttccgccagcttttcaacaccttcttcgttatgcgcaccgtctaaatataccggaggatgttcctgaacaagctctaaccgtcccggccaagcagccttcacaagcccgctccttaacgcttcgtcactgatatgggcgatattctccttattgagccactccgcagccaaaatggacaaagcagcattttgtctttgatgggtgccaatcagagatgtccgaatatcttcatagcatttctcttccgttttgaatgaaaactgttctcctgcaggcagagcctcttcattgaaaataacacatgcatcatgcaatgactggaacggcgcagcatgccgttcggcttcatggcggatgacctgtaaagcttccggctgggtaactgctgtaacgattggaataccttctttaataatgccggccttttctcctgcaatttcttcaatggtgtttcccaaaatgttcatatgatcgtgtccgatgcttgtaatcacagttaagagcggttcaaccacattagtagaatcgaatctaccgcccagacctgtttcaaaaataacaaaatcgaccttatgaaactctgcaaaatataaaaatgcacaagctgtcataatttcaaattctgtcggctgtccgtattccgtttgatcaagggcttcaacgtgcggtttcatttgattgacgagtgctgtccattcctcatctgaaatcggtatcccgtttacgctgatccgttcattaaacgtaataatataaggcgatgtaaatgttccaaccgtatatccggcttcctgcagcatagaacggataaaagcgacagttgatcctttaccgtttgttcctgcgacgtggaacgctcggatttttttttcaggatgtcctaaccgcgccatcagctgtttcattcgaccaagtccgggcttcaccccgaatttcagcctcccgtgaatccagctgcgcgcatcttgatatgcagtaaacaa

SEQ ID NO: 6 - DNA, 507 bp, Bacillus subtilis
atgatttcattcatttttgcgatggatgccaacaggcttatcggcaaagacaatgatttgccgtggcatttgcccaatgatcttgcatactttaagaaaataacatcgggccattcaatcattatgggccggaaaacatttgaatcgatcggacgtccgcttccaaatcggaaaaatattgtcgttacctcagcgccggattcagaatttcagggatgcacggttgtcagttcattaaaggatgtactggacatttgttcaggccctgaagaatgctttgtgatcggaggggctcagctctatacggacctgttcccttatgcggacagactgtatatgacgaaaattcatcacgagtttgagggtgaccgtcactttcctgaatttgatgaatccaattggaagctggtttcttctgagcaggggaccaaagacgaaaaaaacccgtatgattacgaatttctaatgtatgaaaaaaagaattcttctaaagcgggaggattttaa

SEQ ID NO: 7 - protein, 190 aa, Bacillus subtilis
MKEVNKEQIEQAVRQILEAIGEDPNREGLLDTPKRVAKMYAEVFSGLNEDPKEHFQTIFGENHEELVLVKDIAFHSMCEHHLVPFYGKAHVAYIPRGGKVTGLSKLARAVEAVAKRPQLQERITSTIAESIVETLDPHGVMVVVEAEHMCMTMRGVRKPGAKTVTSAVRGVFKDDAAARAEVLEHIKRQD

SEQ ID NO: 8 - protein, 120 aa, Bacillus subtilis
MDKVYVEGMEFYGYHGVFTEENKLGQRFKVDLTAELDLSKAGQTDDLEQTINYAELYHVCKDIVEGEPVKLVETLAERIAGTVLGKFQPVQQCTVKVIKPDPPIPGHYKSVAIEITRKKS

SEQ ID NO: 9 - protein, 167 aa, Bacillus subtilis
MNNIAYIALGSNIGDRETYLRQAVALLHQHAAVTVTKVSSIYETDPVGYEDQAQFLNMAVEIKTSLNPFELLELTQQIENELGRTREVRWGPRTADLDILLFNRENIETEQLIVPHPRMYERLFVLAPLAEICQQVEKEATSAETDQEGVRVWKQKSGVDEFVHSES

SEQ ID NO: 10 - protein, 285 aa, Bacillus subtilis
MAQHTIDQTQVIHTKPSALSYKEKTLVMGILNVTPDSFSDGGKYDSLDKALLHAKEMIDDGAHIIDIGGESTRPGAECVSEDEEMSRVIPVIERITKELGVPISVDTYKASVADEAVKAGASIINDIWGAKHDPKMASVAAEHNVPIVLMHNRPERNYNDLLPDMLSDLMESVKIAVEAGVDEKNIILDPGIGFAKTYHDNLAVMNKLEIFSGLGYPVLLATSRKRFIGRVLDLPPEERAEGTGATVCLGIQKGCDIVRVHDVKQIARMAKMMDAMLNKGGVHHG

SEQ ID NO: 11 - protein, 430 aa, Bacillus subtilis
MFTAYQDARSWIHGRLKFGVKPGLGRMKQLMARLGHPEKKIRAFHVAGTNGKGSTVAFIRSMLQEAGYTVGTFTSPYIITFNERISVNGIPISDEEWTALVNQMKPHVEALDQTEYGQPTEFEIMTACAFLYFAEFHKVDFVIFETGLGGRFDSTNVVEPLLTVITSIGHDHMNILGNTIEEIAGEKAGIIKEGIPIVTAVTQPEALQVIRHEAERHAAPFQSLHDACVIFNEEALPAGEQFSFKTEEKCYEDIRTSLIGTHQRQNAALSILAAEWLNKENIAHISDEALRSGLVKAAWPGRLELVQEHPPVYLDGAHNEEGVEKLAETMKQRFANSRISVVFSALKDKPYQNMIKRLETIAHAIHFASFDFPRASLAKDLYDASEISNKSWSEDPDDVIKFIESKKGSNEIVLITGSLYFISDIRKRLK

SEQ ID NO: 12 - protein, 168 aa, Bacillus subtilis
MISFIFAMDANRLIGKDNDLPWHLPNDLAYFKKITSGHSIIMGRKTFESIGRPLPNRKNIVVTSAPDSEFQGCTVVSSLKDVLDICSGPEECFVIGGAQLYTDLFPYADRLYMTKIHHEFEGDRHFPEFDESNWKLVSSEQGTKDEKNPYDYEFLMYEKKNSSKAGGF

SEQ ID NO: 13 - DNA, 683 bp, Bacillus subtilis
cgcagcatacgcagcgaaatcagcatcaccggagaatcccaacgaagccaactagtatgaaagaagtcaataaagaacaaattgaacaggcagtgagacagattcttgaagcaattggagaagatccgaacagagagggcttacttgatacaccgaaaagagttgctaaaatgtatgcggaagtcttttcaggcttaaatgaagatccgaaagagcattttcagacaattttcggagaaaaccatgaagagctggtccttgtgaaagatattgcgtttcactcaatgtgcgaacatcacctggtgccgttttacggcaaggcacacgttgcgtatattcctagaggcggaaaagttacaggcttgtcaaaattagcacgcgcagttgaagctgttgcaaaaagaccgcaattacaggaacgcattacatctacaattgcggaatcaattgtcgagacattagaccctcatggcgttatggttgtcgttgaagctgaacacatgtgcatgacaatgcgcggcgtcagaaaacctggcgcaaaaacagtcacatcagcagtcagaggcgtgtttaaagatgatgcggcagctcgtgcggaagtcctggaacatattaaacgccaggactgaaaaagaggggagggaaacattaatgacgacctggctaacgagtctcgccgatct

SEQ ID NO: 14 - DNA, 454 bp, Bacillus subtilis
ttctttttgcgccaggtagccatagctggtcatatgatggataaagtttatgtggaaggaatggaattttatggctatcatggcgtcttcacagaagagaacaaattgggacaacgcttcaaagtagatctgacagcagaactggatttatcaaaagcaggacaaacagacgaccttgaacagacaattaactatgcagagctttaccatgtctgtaaagacattgtcgaaggcgagccggtcaaattggtagagacccttgctgagcggatagctggcacagttttaggtaaatttcagccggttcaacaatgtacggtgaaagttattaaaccagatccgccgattcctggccactataaatcagtagcaattgaaattacgagaaaaaagtcataaattaattctagagtcgatccccgggttcgccagcaatgactaccggcagcccgcc

SEQ ID NO: 15 - DNA, 595 bp, Bacillus subtilis
ggcggggcttcttttatcgaatccagcgtgacatatgatgaacaacattgcgtacattgcgcttggctctaatattggagatagagaaacgtatctgcgccaggccgttgcgttactgcatcaacatgctgcggtcacagttacaaaagtcagctcaatttatgaaacagatccggtcggctatgaagaccaagcccagtttttaaatatggcggttgaaattaaaacaagcctgaatccgtttgaacttctggaactgacacagcaaatcgaaaacgaactgggccgcacacgcgaagttagatggggcccgagaacagcggatttagacattctgctgtttaacagagaaaacattgaaacagagcagttaattgtcccgcatcctcgcatgtatgaacgcctgtttgttcttgcgccgcttgcggaaatttgccagcaggtcgagaaagaagcgacaagcgcggaaacggatcaagaaggagttagagtttggaaacaaaaatcaggcgttgacgaatttgtacatagcgaaagctgaaaaagaggggagggaaacattaatgaccgaccctcatggaaacccttcctggcg

SEQ ID NO: 16 - DNA, 948 bp, Bacillus subtilis
gaccgaccctcatggaaacccttcctggcgcatatgatggcgcagcacacaatagatcaaacacaagtcattcatacgaaaccgagcgcgctttcatataaagaaaaaacactggtcatgggcattcttaacgttacacctgattcttttagcgatggtggaaaatatgacagcttggacaaggcgcttctgcatgccaaagaaatgatcgacgacggcgcgcacattattgacataggaggcgagagcacaagaccgggagctgaatgcgtcagcgaagacgaagaaatgtctcgggtcattccggtcattgaacgcatcacaaaggaactcggcgtcccgatttcagtggatacatataaagcatctgtggcagacgaagcagtcaaagcgggcgcatctattatcaatgacatttggggagcgaaacatgatccgaagatggcaagcgtcgcagcggaacataacgttccaattgtcctgatgcacaatcggccagaacggaattataacgaccttcttccggatatgctgagcgaccttatggaatcagtcaaaattgcggttgaggcgggcgtggatgagaaaaatattattttagatccgggcatcggcttcgcgaagacataccatgataatcttgcagtgatgaataagttagagatcttcagcggacttggctatcctgtcctgctggctacatctcgtaaaagatttatcggaagagttcttgatttaccgcctgaagagagagcagagggcacaggagcgacagtctgcttgggcattcagaaaggatgcgacatagtgcgtgttcatgatgtcaagcaaattgccagaatggcgaaaatgatggacgcgatgctgaataagggaggggtgcaccatggatgaaaaagaggggagggaaacattaatttctttttgcgccaggtagccatagctggt

SEQ ID NO: 17 - DNA, 598 bp, Bacillus subtilis
gacgacctggctaacgagtctcgccgatctcatatgatgatttcatttattttcgcaatggacgcgaatagactgataggcaaagacaatgatctgccgtggcatttaccgaatgacctggcttattttaaaaaaattacaagcggccatagcatcattatgggacgtaaaacatttgagtcaattggcagacctcttccgaacagaaaaaacattgttgtcacatctgcgccggattcagaatttcagggctgcacagtcgtctcaagccttaaagacgttcttgatatttgcagcggaccggaagagtgttttgtcattggcggagcgcaattatacacagatctttttccgtacgcggatagactgtatatgacaaaaatccaccatgaatttgaaggcgacagacactttcctgaatttgacgagagcaactggaaactcgtgtctagcgaacagggcacgaaggatgagaaaaatccgtatgactatgaatttcttatgtatgaaaagaaaaacagcagcaaagcgggaggcttttgaaaaagaggggagggaaacattaatggcggggcttcttttatcgaatccagcgtga

SEQ ID NO: 18 - DNA, 1460 bp, Artificial sequence
  DNA fragments
gccttttaatcccggcaacagcttaatcagtacatccatcattccgaagcatccgacattcgatcattacaaggaattatttgcgggcaaggaaagccttcaatatgtgcagtggtatgtcaactctatgaagatcagcctgtttacaatggcagggtctttgctctgtgtgacgtttacggcctatgcgttttcgcgctttcggtttaaagggaggaaatacgctttaacgctctttttattgctgcagatgattcctcagttttcagctttaattgccttgtttgtgctggcgcaaatcttgggaatgatcaatagccactggctgctaatcttgctttatatcggcggcctgatcccgatgaatacgtatttgatgaaagggtacatggattccattccgatggatttagacgaaagcgccaagattgacggagccagcagcaccagaatcttcttccagatcattctgccattatcaaaaccgatggcggcagtcgtggccatgaacggctttaccggtccgctcggagattttgtgctgtcctcaaccatattgagaacgcctgaatcatatacattgcccgtcggtctattcaatttagtgaatgatgtcatgggggccagctatacgacatttgcggccggagccctgcttatcagcataccggttgccgtcatctttattatgctgcaaaagaattttgtgtccggattaaccgcaggcggaacgaagggctaagagaacaaggaggagaatgtgatgtcaaagcttgaaaaaacgcacgtaacaaaagcaaaatttatgctccatgggggagactacaaccccgatcagtggctggatcggcccgatattttagctgacgatatcaaactgatgaagctttctcatacgaatacgttttctgtcggcatttttgcatggagcgcacttgagccggaggagggcgtatatcaatttgaatggctggatgatatttttgagcggattcacagtataggcggccgggtcatattagcaacgccgagcggagcccgtccggcctggctgtcgcaaacctatccggaagttttgcgcgtcaatgcctcccgcgtcaaacagctgcacggcggaaggcacaaccactgcctcacatctaaagtctaccgagaaaaaacacggcacatcaaccgctacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagttggcttcgttgggattctccggtgatgctgatttcgctgcgtatgctgcg

SEQ ID NO: 19 - DNA, 1038 bp, Artificial sequence
  DNA fragments
tctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcc

SEQ ID NO: 20 - DNA, 5390 bp, artificial sequence
  expression cassette
tcccggcaacagcttaatcagtacatccatcattccgaagcatccgacattcgatcattacaaggaattatttgcgggcaaggaaagccttcaatatgtgcagtggtatgtcaactctatgaagatcagcctgtttacaatggcagggtctttgctctgtgtgacgtttacggcctatgcgttttcgcgctttcggtttaaagggaggaaatacgctttaacgctctttttattgctgcagatgattcctcagttttcagctttaattgccttgtttgtgctggcgcaaatcttgggaatgatcaatagccactggctgctaatcttgctttatatcggcggcctgatcccgatgaatacgtatttgatgaaagggtacatggattccattccgatggatttagacgaaagcgccaagattgacggagccagcagcaccagaatcttcttccagatcattctgccattatcaaaaccgatggcggcagtcgtggccatgaacggctttaccggtccgctcggagattttgtgctgtcctcaaccatattgagaacgcctgaatcatatacattgcccgtcggtctattcaatttagtgaatgatgtcatgggggccagctatacgacatttgcggccggagccctgcttatcagcataccggttgccgtcatctttattatgctgcaaaagaattttgtgtccggattaaccgcaggcggaacgaagggctaagagaacaaggaggagaatgtgatgtcaaagcttgaaaaaacgcacgtaacaaaagcaaaatttatgctccatgggggagactacaaccccgatcagtggctggatcggcccgatattttagctgacgatatcaaactgatgaagctttctcatacgaatacgttttctgtcggcatttttgcatggagcgcacttgagccggaggagggcgtatatcaatttgaatggctggatgatatttttgagcggattcacagtataggcggccgggtcatattagcaacgccgagcggagcccgtccggcctggctgtcgcaaacctatccggaagttttgcgcgtcaatgcctcccgcgtcaaacagctgcacggcggaaggcacaaccactgcctcacatctaaagtctaccgagaaaaaacacggcacatcaaccgctacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagttggcttcgttgggattctccggtgatgctgatttcgctgcgtatgctgcgatgaaagaagtcaataaagaacaaattgaacaggcagtgagacagattcttgaagcaattggagaagatccgaacagagagggcttacttgatacaccgaaaagagttgctaaaatgtatgcggaagtcttttcaggcttaaatgaagatccgaaagagcattttcagacaattttcggagaaaaccatgaagagctggtccttgtgaaagatattgcgtttcactcaatgtgcgaacatcacctggtgccgttttacggcaaggcacacgttgcgtatattcctagaggcggaaaagttacaggcttgtcaaaattagcacgcgcagttgaagctgttgcaaaaagaccgcaattacaggaacgcattacatctacaattgcggaatcaattgtcgagacattagaccctcatggcgttatggttgtcgttgaagctgaacacatgtgcatgacaatgcgcggcgtcagaaaacctggcgcaaaaacagtcacatcagcagtcagaggcgtgtttaaagatgatgcggcagctcgtgcggaagtcctggaacatattaaacgccaggactgaaaaagaggggagggaaacattatgatgatttcatttattttcgcaatggacgcgaatagactgataggcaaagacaatgatctgccgtggcatttaccgaatgacctggcttattttaaaaaaattacaagcggccatagcatcattatgggacgtaaaacatttgagtcaattggcagacctcttccgaacagaaaaaacattgttgtcacatctgcgccggattcagaatttcagggctgcacagtcgtctcaagccttaaagacgttcttgatatttgcagcggaccggaagagtgttttgtcattggcggagcgcaattatacacagatctttttccgtacgcggatagactgtatatgacaaaaatccaccatgaatttgaaggcgacagacactttcctgaatttgacgagagcaactggaaactcgtgtctagcgaacagggcacgaaggatgagaaaaatccgtatgactatgaatttcttatgtatgaaaagaaaaacagcagcaaagcgggaggcttttgaaaaagaggggagggaaacattatgatgaacaacattgcgtacattgcgcttggctctaatattggagatagagaaacgtatctgcgccaggccgttgcgttactgcatcaacatgctgcggtcacagttacaaaagtcagctcaatttatgaaacagatccggtcggctatgaagaccaagcccagtttttaaatatggcggttgaaattaaaacaagcctgaatccgtttgaacttctggaactgacacagcaaatcgaaaacgaactgggccgcacacgcgaagttagatggggcccgagaacagcggatttagacattctgctgtttaacagagaaaacattgaaacagagcagttaattgtcccgcatcctcgcatgtatgaacgcctgtttgttcttgcgccgcttgcggaaatttgccagcaggtcgagaaagaagcgacaagcgcggaaacggatcaagaaggagttagagtttggaaacaaaaatcaggcgttgacgaatttgtacatagcgaaagctgaaaaagaggggagggaaacattatgatggcgcagcacacaatagatcaaacacaagtcattcatacgaaaccgagcgcgctttcatataaagaaaaaacactggtcatgggcattcttaacgttacacctgattcttttagcgatggtggaaaatatgacagcttggacaaggcgcttctgcatgccaaagaaatgatcgacgacggcgcgcacattattgacataggaggcgagagcacaagaccgggagctgaatgcgtcagcgaagacgaagaaatgtctcgggtcattccggtcattgaacgcatcacaaaggaactcggcgtcccgatttcagtggatacatataaagcatctgtggcagacgaagcagtcaaagcgggcgcatctattatcaatgacatttggggagcgaaacatgatccgaagatggcaagcgtcgcagcggaacataacgttccaattgtcctgatgcacaatcggccagaacggaattataacgaccttcttccggatatgctgagcgaccttatggaatcagtcaaaattgcggttgaggcgggcgtggatgagaaaaatattattttagatccgggcatcggcttcgcgaagacataccatgataatcttgcagtgatgaataagttagagatcttcagcggacttggctatcctgtcctgctggctacatctcgtaaaagatttatcggaagagttcttgatttaccgcctgaagagagagcagagggcacaggagcgacagtctgcttgggcattcagaaaggatgcgacatagtgcgtgttcatgatgtcaagcaaattgccagaatggcgaaaatgatggacgcgatgctgaataagggaggggtgcaccatggatgaaaaagaggggagggaaacattatgatggataaagtttatgtggaaggaatggaattttatggctatcatggcgtcttcacagaagagaacaaattgggacaacgcttcaaagtagatctgacagcagaactggatttatcaaaagcaggacaaacagacgaccttgaacagacaattaactatgcagagctttaccatgtctgtaaagacattgtcgaaggcgagccggtcaaattggtagagacccttgctgagcggatagctggcacagttttaggtaaatttcagccggttcaacaatgtacggtgaaagttattaaaccagatccgccgattcctggccactataaatcagtagcaattgaaattacgagaaaaaagtcataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagat

SEQ ID NO: 21 - DNA, 2124 bp, artificial sequence
  Tetracycline resistance cassette
aattcttactgcagatagtgtacgtaaaaagattaaattattgcttggtgaaaaaagtcttgcaatggtgcaggttgttctcaatgtcgaaaatatgtatttgtatttaacgcacgagagcaaggacgctattgctaagaagaaacatgtttatgataaggctgatataaagctaatcaataattttgatattgaccgttatgtgacgttagatgtcgaggaaaagaccgaacttttcaatgtggttgtatcgcttattcgtgcgtacactctccaaaatatttttgatttgtatgatttcattgacgaaaatggagaaacttatgggttgactataaatttggttaacgaagttattgcagggaaaactggttttatgaaattgttgtttgacggagcttatcaacgtagtaagcgtggaacaaagaacgaagagagataaaaagttgatctttgtgaaaactacagaaagtaaagaatgaaaagagtaatgctaacatagcattacggattttatgaccgatgatgaagaaaagaatttgaaacttagtttatatgtggtaaaatgttttaatcaagtttaggaggaattaattatgaagtgtaattaatgtaacagggttcaattaaaagagggaagcgtatcattaaccctataaactacgtctgccctcattattggagggtgaaatgtgaatacatcctattcacaatcgaatttacgacacaaccaaattttaatttggctttgcattttatctttttttagcgtattaaatgaaatggttttgaacgtctcattacctgatattgcaaatgattttaataaaccacctgcgagtacaaactgggtgaacacagcctttatgttaaccttttccattggaacagctgtatatggaaagctatctgatcaattaggcatcaaaaggttactcctatttggaattataataaattgtttcgggtcggtaattgggtttgttggccattctttcttttccttacttattatggctcgttttattcaaggggctggtgcagctgcatttccagcactcgtaatggttgtagttgcgcgctatattccaaaggaaaataggggtaaagcatttggtcttattggatcgatagtagccatgggagaaggagtcggtccagcgattggtggaatgatagcccattatattcattggtcctatcttctactcattcctatgataacaattatcactgttccgtttcttatgaaattattaaagaaagaagtaaggataaaaggtcattttgatatcaaaggaattatactaatgtctgtaggcattgtattttttatgttgtttacaacatcatatagcatttcttttcttatcgttagcgtgctgtcattcctgatatttgtaaaacatatcaggaaagtaacagatccttttgttgatcccggattagggaaaaatataccttttatgattggagttctttgtgggggaattatatttggaacagtagcagggtttgtctctatggttccttatatgatgaaagatgttcaccagctaagtactgccgaaatcggaagtgtaattattttccctggaacaatgagtgtcattattttcggctacattggtgggatacttgttgatagaagaggtcctttatacgtgttaaacatcggagttacatttctttctgttagctttttaactgcttcctttcttttagaaacaacatcatggttcatgacaattataatcgtatttgttttaggtgggctttcgttcaccaaaacagttatatcaacaattgtttcaagtagcttgaaacagcaggaagctggtgctggaatgagtttgcttaactttaccagctttttatcagagggaacaggtattgcaattgtaggtggtttattatccatacccttacttgatcaaaggttgttacctatggaagttgatcagtcaacttatctgtatagtaatttgttattacttttttcaggaatcattgtcattagttggctggttaccttgaatgtatataaacattctcaaagggatttctaaatcgttaagggatcaactttgggagagagttcaaaattgatcctttctgcagaag

SEQ ID NO: 22 - protein, 419 aa, Lactobacillus reuteri
MRTYEQINAGFNRQMLGGQRDRVKFLRRILTRLGNPDQRFKIIHIAGTNGKGSTGTMLEQGLQNAGYRVGYFSSPALVDDREQIKVNDHLISKKDFAMTYQKITEHLPADLLPDDITIFEWWTLIMLQYFADQKVDWAVIECGLGGQDDATNIISAPFISVITHIALDHTRILGPTIAKIAQAKAGIIKTGTKQVFLAPHQEKDALTIIREKAQQQKVGLTQADAQSIVDGKAILKVNHKIYKVPFNLLGTFQSENLGTVVSVFNFLYQRRLVTSWQPLLSTLATVKIAGRMQKIADHPPIILDGAHNPDAAKQLTKTISKLPHNKVIMVLGFLADKNISQMVKIYQQMADEIIITTPDHPTRALDASALKSVLPQAIIANNPRQGLVVAKKIAEPNDLIIVTGSFYTIKDIEANLDEK

SEQ ID NO: 23 - protein, 406 aa, Ashbya gossypii
MELGLGRITQVLRQLHSPHERMRVLHVAGTNGKGSVCAYLAAVLRAGGERVGRFTSPHLVHPRDAITVDGEVIGAATYAALKAEVVAAGTCTEFEAQTAVALTHFARLECTWCVVEVGVGGRLDATNVVPGGRKLCAITKVGLDHQALLGGTLAVVAREKAGIVVPGVRFVAVDGTNAPSVLAEVRAAAAKVGAEVHETGGAPVCTVSWGAVAASALPLAGAYQVQNAGVALALLDHLQQLGEISVSHAALERGLKAVEWPGRLQQVEYDLGGVHVPLLFDGAHNPCAAEELARFLNERYRGPGGSPLIYVLAVTCGKEIDALLAPLLKPHDRVFATSFGAVESMPWVAAMASEDVAAAARRYTAHVSAVADPLDALRAAAAARGDANLVVCGSLYLVGELLRREH

SEQ ID NO: 24 - DNA, 1399 bp, Lactobacillus reuteri
ttttactagtatgagaacatacgaacaaattaatgcaggatttaatcgccagatgctgggcggccagagagacagagtcaagttccttagacgcatccttacgagacttggaaaccctgatcagcgctttaaaattattcatatcgcgggaacgaacggcaaaggatcaacaggcactatgttagaacagggccttcagaatgcgggataccgcgtcggctactttagctctcctgcgctggttgatgatcgcgaacaaattaaagtcaatgatcaccttatcagcaagaaagattttgcgatgacctatcagaaaattacggagcatctgcctgctgaccttctgcctgatgatattacaatctttgagtggtggacgttaatcatgcttcaatactttgcggatcaaaaggttgactgggcggtgattgaatgtggtcttggcggccaagacgatgcgacaaacatcatctcagcgccgttcatttcagtcattacccatatcgctcttgaccacacccgtatcctgggccctacaattgcgaagattgcgcaagctaaggcaggcattataaagacagggactaaacaggttttcctggcaccacatcaagagaaggatgcgttaacaatcattcgcgaaaaagcgcaacagcaaaaggtcggactgacgcaggcagatgcacagagcattgtggacggaaaagctattttaaaagtgaatcacaagatttacaaggtcccttttaatctgctgggcacatttcagtcagaaaacctgggaacggttgttagcgtctttaactttctgtatcagcgccgtcttgtcacgtcatggcaaccgttacttagcacactggcaacagttaaaattgcaggaagaatgcaaaaaatcgcggatcatcctccgatcattcttgatggcgcacataatccggatgctgcaaagcagcttacaaagacaattagcaaactcccacataataaagtcataatggtgttaggcttccttgctgacaaaaacatttcacagatggtcaagatttaccaacagatggcggatgaaattatcattacaacgcctgaccatcctacaagagcgctggacgcctcagcccttaaatcagtcttaccgcaagcaattattgcgaataatcctcgtcagggactggttgttgctaagaaaattgcagagccgaacgatcttatcatcgtcacgggcagcttctacacaatcaaggatattgaggcaaatttagatgagaaataagcagaggctgtgatcagtctctgcttttttttctgcgttctatttctttttcacgttcacggatgacgtcagtccgatcccgcaaacggtgtttgtcgataagaaatatgaattcgcgtgcgcattgga

SEQ ID NO: 25 - DNA, 1360 bp, Ashbya gossypii
ttttactagtatggagttaggcttaggccgcatcacacaagtgctgagacaattacatagccctcatgaaagaatgcgtgtcttacatgttgcaggaacaaatggcaaaggaagcgtctgtgcgtatttagcggctgttttaagagcgggcggagaaagagttggcagatttacaagccctcacttagttcatccgcgcgatgctatcacagtcgacggcgaagttattggagcggcgacatatgctgcacttaaagctgaagtcgttgcggcaggcacatgcacggagtttgaagcacaaacggcggttgcgcttacgcattttgcaagacttgaatgcacatggtgtgtcgtcgaagtgggcgtcggcggcagattagacgctacaaatgtcgtccctggcggacgcaaactgtgtgcaattacaaaggttggattagatcatcaggcgttacttggcggaacactggctgttgttgcaagagagaaggccggcattgtggttccgggagtgcgctttgtcgctgtcgacggcacgaacgcaccttcagttctggcggaggttcgggcggctgcagcgaaagttggcgcagaggtccatgagacaggaggcgcgccggtttgcacagtcagctggggtgcggttgctgcaagcgcacttccgttagcgggagcttaccaggtacaaaacgcgggcgttgcacttgcactgcttgatcatcttcaacaactgggagagatctcagtcagccatgcagcactggaaagaggactgaaagcagtcgaatggcctggcagacttcaacaagttgagtatgaccttggaggcgtccatgtcccgctgttatttgacggagcacacaatccgtgtgcagcggaagagcttgcaagattcttaaacgagagataccgcggaccgggaggatctccgctgatctatgtgctggctgtcacgtgtggcaaagagatcgacgcacttcttgcacctcttctgaaaccgcacgatagagtcttcgcaaccagctttggcgcggttgagtctatgccgtgggtcgcagcgatggcaagcgaggatgtcgcagcggcggcgagacgctacacagcccacgtttcagcggttgcggacccgctggacgcgttacgcgccgcagcggcagcacgcggcgatgctaatctggtcgtctgcggatcattatatcttgtcggcgaacttctgcgccgcgaacattaagcagaggctgtgatcagtctctgcttttttttctgcgttctatttctttttcacgttcacggatgacgtcagtccgatcccgcaaacggtgtttgtcgataagaaatatgaattcgcgtgcgcattgga

SEQ ID NO: 26 - DNA, 20 bp, artificial sequence
  primer pair
gcagcgaaatcagcatcacc

SEQ ID NO: 27 - DNA, 20 bp, artificial sequence
  primer pair
gactcgttagccaggtcgtc

SEQ ID NO: 28 - DNA, 20 bp, artificial sequence
  primer pair
tcgataaaagaagccccgcc

SEQ ID NO: 29 - DNA, 20 bp, artificial sequence
  primer pair
ggtttccatgagggtcggtc

SEQ ID NO: 30 - DNA, 20 bp, artificial sequence
  primer pair
gctacctggcgcaaaaagaa

SEQ ID NO: 31 - DNA, 20 bp, artificial sequence
  primer pair
cggtagtcattgctggcgaa

SEQ ID NO: 32 - DNA, 19 bp, artificial sequence
  primer pair
acgacctggctaacgagtc

SEQ ID NO: 33 - DNA, 20 bp, artificial sequence
  primer pair
ggcggggcttcttttatcga

SEQ ID NO: 34 - DNA, 20 bp, artificial sequence
  primer pair
gaccgaccctcatggaaacc

SEQ ID NO: 35 - DNA, 20 bp, artificial sequence
  primer pair
ttctttttgcgccaggtagc

SEQ ID NO: 36 - DNA, 20 bp, artificial sequence
  primer
ggagaatcccaacgaagcca

SEQ ID NO: 37 - DNA, 152 bp, artificial sequence
  promotor
gcatcactatctgcagtaaaatcggaattcaattttgtcaaaataattttattgacaacgtcttattaacgttgatataatttaaattttatttgacaaaaatgggctcgtgttgtacaataaatgttactagagtaaaggaggaaactagt

SEQ ID NO: 38 - DNA, 228 bp, artificial sequence
  p15
gtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagt

SEQ ID NO: 39 - DNA, 25 bp, artificial sequence
  primer
atctacattccctttagtaacgtgt

SEQ ID NO: 40 - DNA, 32 bp, artificial sequence
  primer
aaatctagaaattaagaaggagggattcgtca

SEQ ID NO: 41 - DNA, 34 bp, artificial sequence
  primer
aaaggatccatctacattccctttagtaacgtgt

SEQ ID NO: 42 - DNA, 20 bp, artificial sequence
  primer
tcccggcaacagcttaatca

SEQ ID NO: 43 - DNA, 20 bp, artificial sequence
  primer
ggagccgattctctgcgtta

SEQ ID NO: 44 - DNA, 20 bp, artificial sequence
  primer
aaaatgctccctgcggctat

SEQ ID NO: 45 - DNA, 21 bp, artificial sequence
  primer
caatgagaggggttgctatga

SEQ ID NO: 46 - DNA, 20 bp, artificial sequence
  primer
tcgaacggtcaagcacgtta

SEQ ID NO: 47 - DNA, 34 bp, artificial sequence
  primer
tttgctagcatgataattggaatatgggcagaag

SEQ ID NO: 48 - DNA, 34 bp, artificial sequence
  primer
tttgcggccgccctcctcgtcatttcttcaaaag

SEQ ID NO: 49 - DNA, 6975 bp, Lactococcus lactis
atgataattggaatatgggcagaagatgaagcaggtcttatcggtgaagctgataaaatgccttggtctttacctgctgaacaacaacattttaaagaaacaaccatgaatcaagtgattttgatgggacgaaaaacgtttgaaggcatgaataaacgtgtattgccagggagaataagtattattttaactcgcgatgaaacttatcaatcagataatgaaaaagtgctcatcatgcacagccctaaggaagttctagattggtaccataagcaaaataaagacttatttatcacaggaggagctgaaattttagccctttttgaatctgaacttgaattgctctatcgaacagttgttcatgaaaaatttaaaggagatacttattttccaagtacatttgactttggaagatttaagctagtctctgaaaaatttcacgataaagatgagcggaattcttatacttttacaattaaaaaatatgaaaaagtgaaacaaccatgacaaaatcaatttttgggcttttcacagctctcctttgttggattagcattgtcatcgctattcaatgctttagaaaaaaacgttggggtctgggagtattgtttttactcaatgcttttacgaacctcgtaaatacaattcacgctttttctggaactttattttaaaaaataaaaaaagtgccttttaagtaagccaataacacttactttttatgttagtgaaatcaggaataaaataactatgtcaaatacacaaaatccaaatatacattgttctttctgtggaaagagtcaagatgatgtaaaaaaattgattgccggttcagacgtttatatttgtaatgaatgtattgaactttcaactcgaatcttagaagaagaattaagagaagaacaagattcagaaatgcttgaagttaaaacacctaaagaaatgtttgaccatttaaatgaatatgtgataggtcaagaaaaagcaaaacgtgcacttgcagttgccgtttataatcattacaaacgaattaattttgcagcaagtaaaattgctgaagatattgaactacaaaaatcaaatattctattaatcggacctaccggttctggtaagacttttctcgctcaaactttagcgaaatcactcaacgttccatttgcgattgcagatgcgacaagtttaactgaagctggttatgttggagaagacgttgaaaatattctcttaaaacttttacaagcgagtgatttcaatattgaacgtgctgaacgtggaattatctatatcgatgaaattgataaaattgctaaaaaatctgaaaatgtatcaattactcgtgacgtttccggggaaggtgttcaacaagcccttttgaaaattattgaaggaacggtagctagtgttccaccacaaggtggacgtaaacatcctaatcaagaaatgattcaaattgataccaaaaatatcttatttattgttggtggagcttttgacgggattgaagaaattgtcaaacaacgtttaggtgaaaaaattattggttttggtgccaataataaaaaattaaatgacgatgattcttatatgcaagaaattattgccgaggacattcaaaaattcggattaattcctgaatttattggtcgtctgccaattgttgctgctttggaacgtttgaccgaagaggatttgattcaaattttgacagaacctaaaaatgctttgattaaacaatataaacaactccttttatttgataatgttgaacttgaatttgaagatgaagccctcatggcaattgctagaaaagcaattgagcgcaaaacaggagcgcgtggacttcgttcaattattgaagaagtaatgatggatatcatgtttgaagttccaagtcatgaagaaattacaaaagttattattaatgaagcagttgttgacggaaaagctgagccacaaatgattcgagaggccaagaaaaaatgaccataaatacaaataatctgacaataacaatttcagcagcatcaaaaaaacaatatccagaaaatgattggccagaaattgccttagctgggcgttcaaatgtcggtaaatcaagttttattaatactttacttaatcgtaaaaactttgccagaacttctggtcaacctggtaaaacacagttgctcaatttttataatattgatgatcaacttcatttcgttgacgtacctggttacggctacgctcgtgtttctaaaaaggaacgcgaaaaatggggtaaaatgattgaggaatatttgacaacaagagaaaatttaaaagcagttgtcagcttagttgatattcgtcatgaaccctcagaagatgatttgatgatgtatgagtttttgaaatactaccatattccagtgattttagttgcgaccaaagccgataaagttccacgtggtaagtggaataaacatgaatctattatcaaaaaagcaatgaaatttgatagtacagatgattttattatcttttcttctactgataagacaggatttgaagaagcttgggaagcgattttaagatatctctgaaaatagtgctatgaagagattcatagccttttctacacttaaaaagaggaaatatgtacaaaataaaacttaataatataaaatttagggcacatattggtgttctgccagaagaaaaagttctcggacaaaatctcgaaattgatttaatcgtggaaacaaattttgatttttcaggaaaagacgaattagatgaaactttgtcttatgttgatttctatgaggcaacaaaagcagttgtagaatcttcaaaagctgatttaattgaacatgttgcctttgaaattattcaagcagtaaaggctacttcagagcgtatatcaacggttgaagtccatcttagaaaattagccgtaccgattgaaggaatttttgattcagctgaaattgagatgagaggctaaagctggtttttaagataaatattttaaagagatagaagagaaacaaaatcataaaagattatgtctaaatggaggacttatgcaaacaacttacttaagcatgggaagtaatattggtgaccgtcagtattatttacatgaagccattcgtttattgggaaaacaccctaaaattatgattgaaaaagtatcaaatttttatgaaagtactccagtcggcggcgtcaaacaagatgattttactaatttggcattaaaggtggcaacgctacttgaacctttggaattattatcttttattcatgaagttgagttatctttgaaccgtgagcgaaaaattcattgggggccaagaacaattgatattgatattattttctatgacgacttagaaatgcaagtagaaaacttggttattccacataaagaagcttttaatcgtctttttgtcttgaaacctatttttgaacttattgataaagactttaaatattatgcgtcaatagaaaaagcaatagccgaactttcagtaagtgaacaagagctccatgtaataaaagaagaaaaaacaccgagaaatcgtattgaagatgccgttaaagagattctctttgcagtaggtgaaaatccaaatcgagaaggattacttgaaactccagcaagagtagctaaaatgtatgaagaaattctttcgtcacaacgcttaagcaagtttaatgagtataaactttttgaaattgattcttctaaaacggattcaatcgtgttgattaaagatattcctttttattcaatgtgtgagcatcatatgttaccattttttgggaaagctcatgttgcatatattccagctgatggaaaaattattggcttgtcaaaaattccccgtttagttgattatgtttcgcgcaaactctcggttcaagaaaatatcactcatgatattggagatattttgactgatattttgaatcctaaaggagtggcagttcttgttgaaggacgtcatatgtgcgttgaaatgcgtggagtaaaaaaagtaaattctattactaaaacttcttattttttaggtgaatttaaagaaaataatgaaaaaagaatggaatttttagaaagtcttttatgaaaatcttagaacttaatcaagaatctttttctcttaaaaatattatcctaaaatttgatgagttaaatcacaatgaaatgatttctcttcaaaaaaaactttatcgaaatggtagtttgacaagactggctccagactccttgttagtagttttaacaattgatgacttagcaaaattgattaatctttttgaaaatgatgaagataaaaaaatgcttgaagtgatttataagcgtcatcaaatcatttggtcaggtaaaaatttcaattttgatttaactagaaagtcaattgtctattcaatcgtcaatgttacaccagactctttttatgatggaaatccagataatttaaacctctctcatattttaaaaagagtagaagctgatttagaaaatggagcttctgttcttgagctgggagggaaatcatcgaaaccaggatatgacgatattagcccagaagaggaatggaacagactgaaagaacctattcttgagttgaaaaaaaactttcctaaagcgatttttgctgtcgatacggatgaagcttatgtcatggaacgagttttagacgctggggttgatattattaacgatattgatggttttgatacaaatgataaattaaaagtggtagaaaagtatcaaccggctttagttgctatgaataatgggcgagctggttttagttatgctgataatgtttatgaagaacttccattattttttgaaaataaaaaagaagagttacttcaacttggtttaaaagctgagcaaatcgttattgatcctggagttggtttttttaatggagattcaggttcagatagtcttgagcgggttaaagcaactgaaattttaagcagaataggtttacctcttatgattgcaatctctcgtaagtcatttatgggaaaactcttcaatgcccaaggagatgagcggcttttttcaagccttgtcctagaagcgcaaatggttgctgatgggggacggattttgcgtgttcatgatgttaaggagactaaacgtttactcgatgcaattgaaatttataaggaattttaaaaatgaatgaagacctaattgctgaaattcaagctttatctgctattggaagtgaagaaaaattttccgagattattcgattattgaaaaattcgactttagagcttcgggggaaaaagaatccagatttacaattgtcagcaagtgcattagtttttaaaaaacataaactattttttattgaacacccttatcaaaaggagcttttgcttccagcaggtcatgttgaactaggagaaaagccattggaaactgcgattcgtgagttccatgaagaaacaggtttttcagcgtcagaatcaggcaagttggtagatgttaacttgattaatattccttacaacaaaattaagaatgagaaagaacatcaacacattgattttcgttttctattggaactaaaagaaaaagaagcaggccttgctgaattgccttttttccttcttgatagaactgaagctcctgatgaatttaaaaaatattatcaatacaaaagataaagtagaaaaggtcacaaaatgtctatagaagaagcattggaatggatacattcacgtttaaaatttaatattcgcccaggcctaagtcgtgtttcggcccttttagaattgcttggtcatccagaagagtctttgtcaatgattcacgttgctggaacaaatggaaaaggctccacagtcgctttcacacgctcaatctttatgcaggcaggtctgaaggttgcttctttcacaagtcctttcatcaccacttttggtgagcggatgtcgattaatgcactcccgattgctgatgataaattaatttattatgtagaaatgatccaaccacttgttgctgaacttgataaagatgctgaactgactggaattaccgaatttgaaattatcacggcaatggcttttaaatatttctctgatgagcaggttgatttagcggttattgaagttggtttaggtggacttcttgattcaacaaatgtgattaaacctgttgtttctggaattacaacaattggtttagatcatattgatattcttggttcgaccattgaagaaatcgcagctcaaaaggctggaattattaaaccaggaattccagtagttgttggaaatattgaattaaaagcacttcgggttatatgggaagtggctagaaaaaatacagcgcgtgtttatcaatttccatatgattatcgtacggaagtggaagaacacgaacattttaatttcttttctggtcaagaagcaatattggatattgaaaaatctttagttggcttacatcaaatagaaaatgctggtatggctattgaactttctctggtttatgcaagtaaggttgggattgaattgactgaggatgtgattcgctctggaattcgtgaggctttttggccagctcgtatggaaaaattgggtgaaaaaccactcattttactggatggtgctcataatgttcatgcgatgaatcgtttgcttgaaaatcttagctctgagtttccagataaaaaaattacaatcattttttcagccattaccacaaaagatattagtcaaatgataaaaatgcttcaaactgtgaaaaattcgcatctgattttgacaacttttgattatccaaaagctttgaatttgggagattttcaaagattggaagaagaaggggttgaattggctccaagttgggaattagctttagttcgtgcgcaaaaaaatttagctgaagatgatttgttattagttacaggctctctctatttctcatctcaagttcgtgagtttttgaaaaaagagaagtaa

SEQ ID NO: 50 - DNA, 2463 bp, Ashbya gossypii
atgcagtcccttggattcaagtgtttgctgtctcgcaggagcctgagcaggatatcaatctgtacaagaggaatgagtagtgctaacggtggacgaagtaatgatactgtgcatatacagagacaggcactgaaagttgttgctgggcttgacggatggggtcaattgcaggcgcaggatgtgaaattgaccatgaatatgaacacagattttcgtgcttcctcgcagacggatgatctgaagtactccttgaattatgcggtgatttcacgtggggtgcataggttcgttgagggctgtggacggtaccgctctcttggtcacttggccagggaggtaaagaagttttccatgaatgagtatccgggtatccaaactatagaggtgggtgcggaggcggacgcggcccatttgcgatgcggaagtctgggcgtcgtggtgaacagcgatgggcatcgtcctgatgagattttgctttctggaatgaagcttctgacactaataggggtgttcacttttgaacggcgtcggaagcagtacgttgacttgaagctgtcatttccgtggccgaaggaggctggtgaatttccggattgccaggaattattggacgatgttgtgagctatgtagagagagcgaattttaaaacggcagagtctcttgctgagagtgtagctcacgttgttaccttgagagagtattttcagctgcatcgtgggttaccggtaaaagtcaaggtaattaagcttaatgccattactgagactgagggagttggtgtgagctgtgtaagaagtgcggatgaatttacggggaaaccgcccttctgggaagatattccaaacgatcgagcagacgtgtttaaccttcctgtattccagcagccacatgcatctgtcagtgagtggaatcgtgtgtttctggcgtttggatctaatataggggataggtttgctcacattgagcgaagcttacgtctacttgcggaagatcctaaagttaaactacttcgctcgtcgtctctgttcgagagtgaaccaatgtactttaaggagcagtccccgtttatgaatggcgttgtagaagtgcagacacggtatagcccgcacgagttactagagctatgcaaaaggatagaatatgaacatttaaaacgtgtcaaagagtttgataacggccctcgcagcattgatttagatattttattgtaccaaaatgcaaactttgagcatgtggtactgaactccgaggatttagttattcctcatccaaggatgttggagagatcgtttgttttagagcctctctgtgaattgttggctttccatgaagtgcaccccatttcggctgaatctgtccaaagtcacctaaaagaattgtaccgtaaggggaataaggaagacattcttgttaaacttgtacctttgccgggtattccgtcaaatatacctacaacgcgatttctgaagtttagacgggagtatgaggaggatcaatcgacaagcgaattggttcttaggaccaagtcaaatacatatgtcatgggcatcgtgaatgtgacacctgattctttttctgatggatctcctatgtggaatgatgttaatcatttcctcttaaaagtacaaaggatgatccttgacgttttgaagttacatgaaaacgttatcattgatattggaggctgttcgactaggcctggtagtcagcaaccatcagtggaagaagaacttagtcgtactattcccctaataacagcgatcaggggttgcagagatttttcgcaagagaatgtgatcatatctatagacacttacagaagtgctgttgctgaaaaggccataacagcaggggctgatattgtgaacgatatttcaggaggtagttttgatacaaatatgtttaaggttatcagcgcgtatccgaatgttggttatgtgctatcacacataaggggagatatgactaccatgacgagcctgaataagtatgatgatacagttggtttggatggcgttgaagaattcatttacggtaagaaacagcactcagaacggactaaggtgatccggaacatttgtagggaacttgcggagcgataccagcttgcccttgctagcggaattaagcgctggcagattattttggatccgggtattggttttgcgaagaatgctaaacagaacttagatatcatcaagcataccccgtcaattaagggttatagttgtgtgacacatggacaatttgtaaattttgccaaccttcctgtgttgcttgggccttccaggaagaactttattgggactataattcaagaggcacaggtcgagcgaagggactttgcaacggggactattgtaggctcctgtgttggttatgatgcggatatcatcagggtacatgatgtaactaactgtagcaaaagtgctaggttagcggatgagctttataggaaatag

SEQ ID NO: 51 - DNA, 732 bp, Ashbya gossypii
atgtgtcaggggggcagtaaaggactagttaggcaggacacgcccctaaagacgaggcctgtctcgccatatacgctccaggccccagttgaggcggacggactgtcctggccgagtgcaggggcacgtgtgcgggtcgaggagggcacggaggaagaggcggcacgcgcagcccggatagctgatgcagtcaagacgattttgacggagctgggcgaagacgtgacgcgggagggcctgctggacaccccgcaacggtacgccaaagcgatgctgtacttcaccaagggctaccaagacaatattttgaacgatgtgatcaataatgctgtgtttgacgaagatcatgacgagatggtaattgtgcgggatattgagatccattcgctgtgcgagcaccacctggtacccttcttcgggaaggtgcatattggctacatacctcggaggagagtcctcgggttgtcgaagctcgcccggctagcggaaatgtacgcgcgcaggctgcaggtgcaggagcggctgacgaagcagattgcgatggcattgcaggatatactgcgccctagaggagtagccgttgtggtggaggccacgcatatgtgcatggtgtcacggggggtccagaagtccgggtcctcaactgtcacctcgtgtatgctgggctgcttcagagacatgcacaagacccgggaagaattcttgaacctcttgagaaatagaagtgtatag

SEQ ID NO: 52 - DNA, 2484 bp, artificial sequence
  FOL1-AG
ttttactagtatgcaatcactgggctttaaatgtcttctgagcagaagaagcctgagccgcattagcatctgcacgagaggaatgagctcagcgaatggaggaagaagcaatgatacagttcatattcagcgccaggcacttaaggtcgttgcgggccttgatggctggggacagctgcaggcgcaagacgttaagctgacaatgaacatgaacacagactttcgtgcgtcaagccaaacagatgaccttaaatacagccttaattacgctgtgattagccgtggagtccaccgttttgtcgagggatgcggaagataccgtagcctgggacatctggcgagagaggtcaaaaagttttcaatgaatgagtaccctggcattcagaccattgaggtgggtgccgaggcggacgcggcacacctgagatgcggatctttaggcgttgttgtgaatagcgatggacacagacctgatgagatcttattgtcaggcatgaaacttctgacgctgattggagtctttacattcgagcgtcgcagaaagcaatacgtcgatctgaaactgagcttcccgtggcctaaagaagcaggagagttcccggattgtcaggaacttctggatgacgttgtgagctacgtcgagagagcgaacttcaaaacggcagagtctctggcggagtctgtggcacacgtggtcacacttcgcgaatattttcaacttcatcgtggcttgcctgtcaaagtgaaagtcattaagctgaacgcgatcacagaaacggagggcgtcggagttagctgtgtcagatctgccgatgaatttacaggcaagcctccattttgggaagacatcccgaacgatagagcggacgtctttaatttacctgtgttccagcaacctcatgcaagcgtttcagagtggaatagagtgtttctggcgtttggctccaacattggagatagattcgcgcatatcgagagatctttacgtctgcttgctgaagatcctaaagtcaaactgcttagaagcagcagccttttcgaatctgagcctatgtatttcaaggagcagtccccgtttatgaacggagtcgttgaggtccaaacgagatattcaccgcatgaacttttagagttgtgcaaacgtatcgaatatgaacacctgaaacgtgttaaagagtttgataatggcccgcgttcaattgacctggatatcttactgtaccagaacgcgaactttgagcatgtggtccttaattccgaagacctggtaattccgcatcctagaatgctggaacgcagcttcgtgctggagcctttatgcgagctgcttgcgtttcacgaggttcaccctatatcagccgagtcagtgcagagccatctgaaagaattatacagaaaaggcaataaagaggacattttagtcaagttagtccctctgcctggaatcccttctaatattccgacgacgagatttcttaaatttagacgcgaatatgaagaggaccagtctacatcagaattagtcctgcgtacgaaaagcaacacatacgttatgggaattgtcaatgttacgcctgactcatttagcgacggctcacctatgtggaacgacgtcaatcatttccttctgaaggtgcaacgcatgatcctggatgtcctgaaactgcatgagaatgtcattattgatatcggaggctgctctacaagacctggctctcagcaaccgagcgttgaagaagagttatcacgcacgattcctcttattacagctattcgcggctgcagagatttttcacaagagaatgttattatctcaattgacacataccggtcagcggtcgctgagaaagcaattacggcaggagcggatattgttaatgatatttctggcggatctttcgatacaaatatgtttaaagttatttcagcgtatcctaatgtcggctacgttctgtcccatatccgtggcgatatgacaacgatgacgtcactgaacaaatatgatgacacagtcggcttagatggcgttgaggaatttatctatggcaaaaaacaacattcagaacgtacaaaagtcatccgtaacatctgtcgcgaacttgcagaacgctaccagcttgcacttgcttcaggcattaaacgctggcaaattatccttgatcctggcattggcttcgctaaaaatgctaaacaaaacctggatattattaaacacacgccgagcattaaaggatactcatgtgtgacgcatggacaatttgtgaatttcgcgaatttaccggtactgctgggcccgtctcgcaagaatttcatcggcacaattattcaggaggcgcaagtagaacgcagagatttcgcaacaggcacgattgtgggctcatgtgtcggctatgacgctgatattatccgcgttcacgatgtcacgaattgtagcaagagtgcacgcctggcggatgaactgtatcgcaaataaggatccatttt

SEQ ID NO: 53 - DNA, 1090 bp, artificial sequence
  FOL2-AG
tattggatcctatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgtgtcaaggcggaagcaaaggactggttagacaagacacaccgctgaaaacaagacctgtctcaccttatacactgcaagcacctgtcgaagcagacggattaagctggccgagcgcgggcgcgagagttagagtggaagagggaacggaggaagaagcagcgcgcgcggctagaattgcggatgcagtcaaaacaatattaacagagctgggcgaagacgtgacaagagaaggtcttctggacacaccgcagcggtatgcgaaagctatgctgtactttacgaagggataccaagacaacatcctgaacgatgtcattaacaatgcggtttttgacgaggatcatgatgagatggttatcgttcgcgacatagagatacacagcctgtgtgagcatcacctggtcccatttttcggcaaggtccacataggctacattccgagaagacgtgtcctgggactttctaaactggcgcgcttagctgaaatgtacgcacgcagactccaggtccaagaacgtttaaccaaacagatcgcaatggcactgcaagatatccttcgccctagaggcgtggcagtcgttgttgaggctacgcacatgtgcatggtctctcgcggagtgcaaaagagcggatcatcaacggtaacatcatgtatgctgggatgtttcagagacatgcacaagacgagagaggaatttcttaatttacttagaaacagaagcgtttaagcagaggctgtgatcagtctctgcttttttttctgcgttctatttctttttcacgttcacggatgacgtcagtccgatcccgcaaacggtgtttgtcgataagaaatattacgtaatatggcctcgagtttta

SEQ ID NO: 54 - DNA, 25 bp, artificial sequence
  primer
tattggatcctatggttcactgtcc

SEQ ID NO: 55 - DNA, 20 bp, artificial sequence
  primer
gcggtagtggtgcttacgat

SEQ ID NO: 56 - DNA, 23 bp, artificial sequence
  primer
tgcagggtctttattcttcaact

SEQ ID NO: 57 - DNA, 20 bp, artificial sequence
  primer
gcggtagtggtgcttacgat

SEQ ID NO: 58 - DNA, 1038 bp, artificial sequence; identical to SEQ ID NO: 19
  cassette

SEQ ID NO: 59 - DNA, 20 bp, artificial sequence
  primer pair
taggaggcgagagcacaaga

SEQ ID NO: 60 - DNA, 20 bp, artificial sequence
  primer pair
gccgagttcctttgtgatgc

SEQ ID NO: 61 - DNA, 20 bp, artificial sequence
  primer pair
gcccgagaacagcggattta

SEQ ID NO: 62 - DNA, 20 bp, artificial sequence
  primer pair
cgcaagaacaaacaggcgtt

SEQ ID NO: 63 - DNA, 20 bp, artificial sequence
  primer pair
tggcgttatggttgtcgttg

SEQ ID NO: 64 - DNA, 20 bp, artificial sequence
  primer pair
taaacacgcctctgactgct

SEQ ID NO: 65 - DNA, 20 bp, artificial sequence
  primer pair
ggcggagcgcaattatacac

SEQ ID NO: 66 - DNA, 20 bp, artificial sequence
  primer pair
caggaaagtgtctgtcgcct

SEQ ID NO: 67 - DNA, 20 bp, artificial sequence
  primer pair
gattggccgcttacacatgg

SEQ ID NO: 68 - DNA, 20 bp, artificial sequence
  primer pair
aacgtttgggcttctaccga

SEQ ID NO: 69 - DNA, 20 bp, artificial sequence
  primer pair
cagctcgtgtcgtgagatgt

SEQ ID NO: 70 - DNA, 20 bp, artificial sequence
  primer pair
agagtgcccaactgaatgct

SEQ ID NO: 71 - DNA, 21 bp, artificial sequence
  primer pair
gccctgcataaggaatttaac

SEQ ID NO: 72 - DNA, 23 bp, artificial sequence
  primer pair
agcttatggacatacgactgatg

SEQ ID NO: 73 - protein, 406 aa, Ashbya gossypii; identical to SEQ ID NO: 23

SEQ ID NO: 74 - DNA, 1221 bp, Ashbya gossypii
atggagttaggcttaggccgcatcacacaagtgctgagacaattacatagccctcatgaaagaatgcgtgtcttacatgttgcaggaacaaatggcaaaggaagcgtctgtgcgtatttagcggctgttttaagagcgggcggagaaagagttggcagatttacaagccctcacttagttcatccgcgcgatgctatcacagtcgacggcgaagttattggagcggcgacatatgctgcacttaaagctgaagtcgttgcggcaggcacatgcacggagtttgaagcacaaacggcggttgcgcttacgcattttgcaagacttgaatgcacatggtgtgtcgtcgaagtgggcgtcggcggcagattagacgctacaaatgtcgtccctggcggacgcaaactgtgtgcaattacaaaggttggattagatcatcaggcgttacttggcggaacactggctgttgttgcaagagagaaggccggcattgtggttccgggagtgcgctttgtcgctgtcgacggcacgaacgcaccttcagttctggcggaggttcgggcggctgcagcgaaagttggcgcagaggtccatgagacaggaggcgcgccggtttgcacagtcagctggggtgcggttgctgcaagcgcacttccgttagcgggagcttaccaggtacaaaacgcgggcgttgcacttgcactgcttgatcatcttcaacaactgggagagatctcagtcagccatgcagcactggaaagaggactgaaagcagtcgaatggcctggcagacttcaacaagttgagtatgaccttggaggcgtccatgtcccgctgttatttgacggagcacacaatccgtgtgcagcggaagagcttgcaagattcttaaacgagagataccgcggaccgggaggatctccgctgatctatgtgctggctgtcacgtgtggcaaagagatcgacgcacttcttgcacctcttctgaaaccgcacgatagagtcttcgcaaccagctttggcgcggttgagtctatgccgtgggtcgcagcgatggcaagcgaggatgtcgcagcggcggcgagacgctacacagcccacgtttcagcggttgcggacccgctggacgcgttacgcgccgcagcggcagcacgcggcgatgctaatctggtcgtctgcggatcattatatcttgtcggcgaacttctgcgccgcgaacattaa

SEQ ID NO: 75 - protein, 419 aa, Lactobacillus reuteri; identical to SEQ ID NO: 22

SEQ ID NO: 76 - DNA, 1260 bp, Lactobacillus reuteri
atgagaacatacgaacaaattaatgcaggatttaatcgccagatgctgggcggccagagagacagagtcaagttccttagacgcatccttacgagacttggaaaccctgatcagcgctttaaaattattcatatcgcgggaacgaacggcaaaggatcaacaggcactatgttagaacagggccttcagaatgcgggataccgcgtcggctactttagctctcctgcgctggttgatgatcgcgaacaaattaaagtcaatgatcaccttatcagcaagaaagattttgcgatgacctatcagaaaattacggagcatctgcctgctgaccttctgcctgatgatattacaatctttgagtggtggacgttaatcatgcttcaatactttgcggatcaaaaggttgactgggcggtgattgaatgtggtcttggcggccaagacgatgcgacaaacatcatctcagcgccgttcatttcagtcattacccatatcgctcttgaccacacccgtatcctgggccctacaattgcgaagattgcgcaagctaaggcaggcattataaagacagggactaaacaggttttcctggcaccacatcaagagaaggatgcgttaacaatcattcgcgaaaaagcgcaacagcaaaaggtcggactgacgcaggcagatgcacagagcattgtggacggaaaagctattttaaaagtgaatcacaagatttacaaggtcccttttaatctgctgggcacatttcagtcagaaaacctgggaacggttgttagcgtctttaactttctgtatcagcgccgtcttgtcacgtcatggcaaccgttacttagcacactggcaacagttaaaattgcaggaagaatgcaaaaaatcgcggatcatcctccgatcattcttgatggcgcacataatccggatgctgcaaagcagcttacaaagacaattagcaaactcccacataataaagtcataatggtgttaggcttccttgctgacaaaaacatttcacagatggtcaagatttaccaacagatggcggatgaaattatcattacaacgcctgaccatcctacaagagcgctggacgcctcagcccttaaatcagtcttaccgcaagcaattattgcgaataatcctcgtcagggactggttgttgctaagaaaattgcagagccgaacgatcttatcatcgtcacgggcagcttctacacaatcaaggatattgaggcaaatttagatgagaaataa

SEQ ID NO: 77 - protein, 190 aa, Bacillus subtilis; identical to SEQ ID NO: 7

SEQ ID NO: 78 - DNA, 573 bp, Bacillus subtilis
atgaaagaagtcaataaagaacaaattgaacaggcagtgagacagattcttgaagcaattggagaagatccgaacagagagggcttacttgatacaccgaaaagagttgctaaaatgtatgcggaagtcttttcaggcttaaatgaagatccgaaagagcattttcagacaattttcggagaaaaccatgaagagctggtccttgtgaaagatattgcgtttcactcaatgtgcgaacatcacctggtgccgttttacggcaaggcacacgttgcgtatattcctagaggcggaaaagttacaggcttgtcaaaattagcacgcgcagttgaagctgttgcaaaaagaccgcaattacaggaacgcattacatctacaattgcggaatcaattgtcgagacattagaccctcatggcgttatggttgtcgttgaagctgaacacatgtgcatgacaatgcgcggcgtcagaaaacctggcgcaaaaacagtcacatcagcagtcagaggcgtgtttaaagatgatgcggcagctcgtgcggaagtcctggaacatattaaacgccaggactga

SEQ ID NO: 79 - protein, 120 aa, Bacillus subtilis; identical to SEQ ID NO: 8

SEQ ID NO: 80 - DNA, 363 bp, Bacillus subtilis
atggataaagtttatgtggaaggaatggaattttatggctatcatggcgtcttcacagaagagaacaaattgggacaacgcttcaaagtagatctgacagcagaactggatttatcaaaagcaggacaaacagacgaccttgaacagacaattaactatgcagagctttaccatgtctgtaaagacattgtcgaaggcgagccggtcaaattggtagagacccttgctgagcggatagctggcacagttttaggtaaatttcagccggttcaacaatgtacggtgaaagttattaaaccagatccgccgattcctggccactataaatcagtagcaattgaaattacgagaaaaaagtcataa

SEQ ID NO: 81 - protein, 167 aa, Bacillus subtilis; identical to SEQ ID NO: 9

SEQ ID NO: 82 - DNA, 504 bp, Bacillus subtilis
atgaacaacattgcgtacattgcgcttggctctaatattggagatagagaaacgtatctgcgccaggccgttgcgttactgcatcaacatgctgcggtcacagttacaaaagtcagctcaatttatgaaacagatccggtcggctatgaagaccaagcccagtttttaaatatggcggttgaaattaaaacaagcctgaatccgtttgaacttctggaactgacacagcaaatcgaaaacgaactgggccgcacacgcgaagttagatggggcccgagaacagcggatttagacattctgctgtttaacagagaaaacattgaaacagagcagttaattgtcccgcatcctcgcatgtatgaacgcctgtttgttcttgcgccgcttgcggaaatttgccagcaggtcgagaaagaagcgacaagcgcggaaacggatcaagaaggagttagagtttggaaacaaaaatcaggcgttgacgaatttgtacatagcgaaagctga

SEQ ID NO: 83 - protein, 285 aa, Bacillus subtilis; identical to SEQ ID NO: 10

SEQ ID NO: 84 - DNA, 858 bp, Bacillus subtilis
atggcgcagcacacaatagatcaaacacaagtcattcatacgaaaccgagcgcgctttcatataaagaaaaaacactggtcatgggcattcttaacgttacacctgattcttttagcgatggtggaaaatatgacagcttggacaaggcgcttctgcatgccaaagaaatgatcgacgacggcgcgcacattattgacataggaggcgagagcacaagaccgggagctgaatgcgtcagcgaagacgaagaaatgtctcgggtcattccggtcattgaacgcatcacaaaggaactcggcgtcccgatttcagtggatacatataaagcatctgtggcagacgaagcagtcaaagcgggcgcatctattatcaatgacatttggggagcgaaacatgatccgaagatggcaagcgtcgcagcggaacataacgttccaattgtcctgatgcacaatcggccagaacggaattataacgaccttcttccggatatgctgagcgaccttatggaatcagtcaaaattgcggttgaggcgggcgtggatgagaaaaatattattttagatccgggcatcggcttcgcgaagacataccatgataatcttgcagtgatgaataagttagagatcttcagcggacttggctatcctgtcctgctggctacatctcgtaaaagatttatcggaagagttcttgatttaccgcctgaagagagagcagagggcacaggagcgacagtctgcttgggcattcagaaaggatgcgacatagtgcgtgttcatgatgtcaagcaaattgccagaatggcgaaaatgatggacgcgatgctgaataagggaggggtgcaccatggatga

SEQ ID NO: 85 - protein, 168 aa, Bacillus subtilis; identical to SEQ ID NO: 12

SEQ ID NO: 86 - DNA, 507 bp, Bacillus subtilis
atgatttcatttattttcgcaatggacgcgaatagactgataggcaaagacaatgatctgccgtggcatttaccgaatgacctggcttattttaaaaaaattacaagcggccatagcatcattatgggacgtaaaacatttgagtcaattggcagacctcttccgaacagaaaaaacattgttgtcacatctgcgccggattcagaatttcagggctgcacagtcgtctcaagccttaaagacgttcttgatatttgcagcggaccggaagagtgttttgtcattggcggagcgcaattatacacagatctttttccgtacgcggatagactgtatatgacaaaaatccaccatgaatttgaaggcgacagacactttcctgaatttgacgagagcaactggaaactcgtgtctagcgaacagggcacgaaggatgagaaaaatccgtatgactatgaatttcttatgtatgaaaagaaaaacagcagcaaagcgggaggcttttga

(19) United States
(12) Patent Application Publication
(10) Pub. No.: US 2022/0282207 A1
(43) Pub. Date: Sep. 8, 2022

(54) FOLATE PRODUCING STRAIN AND THE PREPARATION AND APPLICATION THEREOF

(71) Applicant: Chifeng Pharmaceutical Co., Ltd., Chifeng, Inner Mongolia (CN)

(72) Inventors: Ming'An SHI, Pudong New Area, Shanghai (CN); Jia SUN, Pudong New Area, Shanghai (CN); Xiangyu SUN, Pudong New Area, Shanghai (CN); Fei SHAO, Pudong New Area, Shanghai (CN); Zhigang CAI, Pudong New Area, Shanghai (CN); Guoyin ZHANG, Pudong New Area, Shanghai (CN); Marko BLAZIC, Ljubljana (SI); Tina KOGEJ, Ljubljana (SI); Gregor KOSEC, Ljubljana (SI); Stefan FUJS, Ljubljana (SI); Alen CUSAK, Ljubljana (SI); Jaka HORVAT, Ljubljana (SI)

(21) Appl. No.: 17/637,443

(22) PCT Filed: May 13, 2020
(86) PCT No.: PCT/CN2020/090084

§ 371 Date: Feb. 22, 2022

(30) Foreign Application Priority Data
Aug. 23, 2019 (CN) PCT/CN2019/102317

(51) Int. Cl. C12N 1/20; C12P 17/18

(52) U.S. Cl. CPC C12N 1/205; C12P 17/182; C12N 2510/02

(57) ABSTRACT

Provided is a folate producing strain and the preparation and application thereof, in particular, the expression level of the endogenous folC gene in the engineered strain of the present invention is decreased, and the exogenous folC gene is introduced, and the production capacity of the folate, the precursor, or the intermediate thereof in the engineered strain is significantly improved compared to the starting strain.

### TECHNICAL FIELD

The invention relates to the field of biotechnology engineering, in particular to folate producing strain and the preparation and application thereof.

### BACKGROUND

Folate is a general term for folic acid and a number of its derivatives; they differ in the state of oxidation, one-carbon substitution of the pteridine ring and in the number of γ-linked glutamate residues (shown in FIG. 1). The pteridine moiety of folates can exist in three oxidation states: fully oxidized (folic acid), or as the reduced 7,8-dihydrofolate (DHF), or 5,6,7,8-tetrahydrofolate (THF) (see structure I). THF is the co-enzymatically active form of the vitamin that accepts, transfers, and donates C1 groups, which are attached either at the N5 or N10 position or by bridging these positions. The C1 groups also differ in their oxidation state, with folates existing as derivatives of formate (5-formyl-THF (5-FTHF or folinic acid), 10-formyl-THF, 5,10-methenyl-THF, and 5-forminino-THF), methanol (5-methyl-THF) or formaldehyde (5,10-methylene-THF). In addition, most naturally occurring folates exist as γ-linked polyglutamate conjugates.

Folic acid (pteroyl-L-glutamic acid) is a synthetic compound, which does not exist in nature. Folic acid is not active as a coenzyme and has to undergo several metabolic steps within the cell to be converted into the metabolically active THF form. However, folic acid is the commercially most important folate compound, produced industrially by chemical synthesis. Mammals cannot synthesize folates and depend on dietary supplementation to maintain normal levels of folates. Low folate status may be caused by low dietary intake, poor absorption of ingested folate and alteration of folate metabolism due to genetic defects or drug interactions. Most countries have established recommended intakes of folate through folic acid supplements or fortified foods. Folates used in diet supplementation include folic acid, folinic acid (5-FTHF, Leucovorin) or 5-MTHF (Scaglione and Panzavolta 2014). Two salt forms of 5-MTHF are currently produced as supplements. Merck Millipore produces Metafolin®, a calcium salt of 5-MTHF, which is a stable crystalline form of the naturally-occurring predominant form of folate. Gnosis S.p.A. developed and patented a glucosamine salt of (6S)-5-MTHF, brand named Quatrefolic®.

Currently, folic acid is industrially primarily produced through chemical synthesis while, unlike other vitamins, microbial production of folic acid on industrial scale is not exploited due to the low yields of folic acid produced by current bacterial strains (Rossi et al., 2016). Although chemically produced folic acid is not a naturally occurring molecule human beings are able to metabolize it into biological active forms of folates by the action of the enzyme dihydrofolate reductase (DHFR). Several reasons support the replacement of chemical synthesis methods by microbial fermentation for commercial production of folates: first, reduced forms of folic acid can be produced by microorganisms, which can be used by humans more efficiently. Most importantly, a single step fermentation process can in principle be much more efficient and environmentally friendly than a multi-stage chemical process.

Previous studies have been done to elucidate folate/folic acid production in microorganisms. Most of microbial application for the production of folates is limited to the fortification of fermented dairy products and to folate-producing probiotics. The optimization of the culture conditions to improve the synthesis of folates have been also carried out, reaching folic acid yields of about 150 μg/g (Hjortmo et al., 2008; Sybesma et al., 2003b). A few studies have described genetically modified strains either of lactic acid bacteria (Sybesma et al., 2003a), yeasts (Walkey et al., 2015) or filamentous fungus (Serrano-Amatriain et al. 2016), which are able to produce folic acid with titers of up to 6.6 mg/L. Another successfully used approach for microbial production of folates is cultivation of yeast or bacterial strains in the presence of para-aminobenzoic acid (pABA). Total folate content of up to 22 mg/L was measured in supernatants of these cultures.

Therefore, there is an urgent need to develop a new folate producing strain for enhancing the production capacity of a folate, a salt, a precursor, or an intermediate thereof.

### SUMMARY OF THE INVENTION

The object of the present invention is to provide a folate producing strain and the preparation and application thereof.

In the first aspect of the present invention, it provides a genetically engineered strain for the synthesis of a folate, a salt thereof, a precursor thereof, or an intermediate thereof, wherein the expression level of the endogenous folC gene in the engineered strain is decreased, and an exogenous folC gene is introduced and the engineered strain has a significantly improved production capacity of a folate, a precursor, or an intermediate thereof compared to its starting strain.

In another preferred embodiment, the structural formula of a folate, a salt, a precursor, or an intermediate thereof is as shown in Formula I:

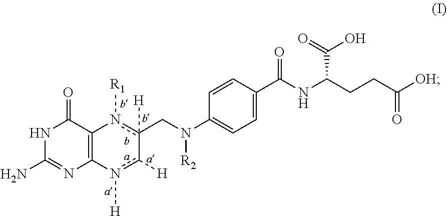

wherein, when a is single bond, a′ is none or when a′ is a single bond, a is none;

when b is a single bond, b′ is none or when b′ is a single bond, b is none;
R1 is selected from the group consisting of: —H, —CH₃ (5-methyl), —CHO (5-formyl), —CH═ or ═CH— (5,10-methenyl), —CH₂— (5,10-methylene), —CH═NH (5-formimino-) and a combination thereof;

R2 is selected from the group consisting of: —H. —CHO (10-formyl), —CH═, ═CH— (5, 10-methenyl), —CH₂— (5,10-methylene) and a combination thereof.

In another preferred embodiment, the starting strain of the engineered strain is selected from the group consisting of Escherichia coli, Lactococcus lactis, Bacillus subtilis, Candida famata and Ashbya gossypii.

In another preferred embodiment, the starting strain of the engineered strain comprises Bacillus subtilis.

In another preferred embodiment, the genetically engineered strain is a bacterium.

In another preferred embodiment, the genetically engineered strain is a bacterium of the genus Bacillus.

In another preferred embodiment, the genetically engineered strain is a bacterium of species Bacillus subtiltis.

In another preferred embodiment, the decreased expression level of the endogenous folC gene means that the expression level of the endogenous folC gene in the engineered strain is reduced by at least 50%, preferably by at least 60%, 70%, 80%, 90%, or 100% compared to the starting strain (wild type).

In another preferred embodiment, the exogenous folC gene is derived from Ashbya gossypii, or Lactobacillus reuteri.

In another preferred embodiment, the expression product of the exogenous folC gene comprises a polypeptide or a derivative polypeptide thereof selected from the group consisting of: dihydrofolate synthase (DHFS-EC 6.3.2.12).

In another preferred embodiment, the amino acid sequence of the dihydrofolate synthase is as shown in SEQ ID NO.: 22 or 23.

In another preferred embodiment, the polynucleotide sequence coding for the dihydrofolate synthase is as shown in SEQ ID NO.: 24 or 25.

In another preferred embodiment, the exogenous folC gene comprises the gene, which is ≥80% identical to the exogenous folC gene, preferably ≥90%, more preferably ≥95%, more preferably, ≥98%, more preferably, ≥99% (note: on the level of nucleotide).

In another preferred embodiment, the exogenous folC gene is shown in SEQ ID NO.:24 or 25.

In another preferred embodiment, the dihydrofolate synthase comprises an amino acid sequence having at least 70%, such as at least 80%, at least 85%, at least 90%, at least 95%, at least 98% or at least 99%, sequence identity with SEQ ID NO: 22 or 23.

In another preferred embodiment, the “significantly improved” means that compared with the starting strain, the fermentation yield of folic acid in the engineered strain is at least more than 0.01 g/L, preferably at least 0.01-0.1 g/L; more preferably, at least 0.1-1 g/L, according to the volume of fermentation broth, per liter; and/or

the “significant improved” means that the folate production capacity in the engineered strain is increased or improved by 100%; preferably by 200-50000%; compared to the starting strain.

In another preferred embodiment, the “significant improved” means that the folate production capacity in the engineered strain is increased or improved by at least 50%, such as at least 100%, at least 200%, at least 500%, at least 1000%, at least 2000%, at least 5000%, at least 10000%, at least 20000% or at least 50000%, compared to the starting strain.

In another preferred embodiment, a gene encoding a folate biosynthetic enzyme is introduced or up-regulated in the engineered strain.

In another preferred embodiment, the up-regulation means that compared with the starting strain (wide type), in the engineered strain that the folate biosynthetic gene is introduced or up-regulated, the expression level of the folate biosynthetic gene has at least a 80% increase, and more preferably, at least 100%, 200%, 300%, 400%, 500%, 600% or 800%.

In another preferred embodiment, the up-regulation means that compared with the starting strain (wide type), in the engineered strain that the folate biosynthetic gene is introduced or up-regulated, the expression level of the folate biosynthetic gene has at least 50%, such as by at least 100%, at least 200%, at least 500%, at least 1000%, at least 2000%, at least 5000%, at least 10000%, at least 20000% or at least 50000%, compared to the starting strain (wide type).

In another preferred embodiment, the folate biosynthetic gene is selected from the group consisting of folE/mtrA, folB, folK, folP/sul, folA/dfrA, and a combination thereof.

In another preferred embodiment, the folate biosynthetic gene is at least one gene (such as at least two, at least three, at least four, or at least five genes) selected from the group consisting of folE/mtrA, folB, folK, folP/sul and folA/dfrA.

In another preferred embodiment, the folate biosynthetic gene is derived from a bacterium or fungus, preferably selected from the genus Bacillus, Lactococcus and Ashbya.

In another preferred embodiment, the folate biosynthetic gene is derived from a bacterium, preferably from a bacterium of the Bacillus species, most preferably from Bacillus subtilis or Lactococcus lactis or Ashbya gossypii.

In another preferred embodiment, the expression product of the folate biosynthetic gene comprises a polypeptide or the derivatives thereof selected from the group consisting of: GTP cyclohydrolase, 7,8-dihydroneopterin aldolase, 2-amino-4-hydroxy-6-hydroxymethyldihydropteridine pyrophosphokinase, dihydropteroate synthase, dihydrofolate reductase, and a combination thereof.

In another preferred embodiment, the expression product of the folate biosynthetic gene is at least one enzyme involved in the biosynthesis of folic acid.

In another preferred embodiment, the at least one enzyme involved in the biosynthesis of folic acid is heterologous to the genetically engineered microorganism.

In another preferred embodiment, the at least one enzyme involved in the biosynthesis of folic acid is derived from a bacterium or fungus, preferably selected from the genus Bacillus, Lactococcus, Shewanella, Vibrio and Ashbya.

In another preferred embodiment, the at least one enzyme involved in the biosynthesis of folic acid is derived from Bacillus subtiltis, Lactobacillus lactis, Shewanella violacea, Vibrio natriegens or Ashbya gossypii.

In another preferred embodiment, the polypeptide having GTP cyclohydrolase activity comprises an amino acid sequence having at least 70%, such as at least 80%, at least 85%, at least 90%, at least 95%, at least 98% or at least 99%, sequence identity with SEQ ID NO: 7.

In another preferred embodiment, the polypeptide having 7,8-dihydroneopterin aldolase activity comprises an amino acid sequence having at least 70%, such as at least 80%, at least 85%, at least 90%, at least 95%, at least 98% or at least 99%, sequence identity with SEQ ID NO: 8.

In another preferred embodiment, the polypeptide having 2-amino-4-hydroxy-6-hydroxymethyl-dihydropteridine pyrophosphokinase activity comprises an amino acid sequence having at least 70%, such as at least 80%, at least 85%, at least 90%, at least 95%, at least 98% or at least 99%, sequence identity with SEQ ID NO: 9.

In another preferred embodiment, the polypeptide having dihydropteroate synthase activity comprises an amino acid sequence having at least 70%, such as at least 80%, at least 85%, at least 90%, at least 95%, at least 98% or at least 99%, sequence identity with SEQ ID NO: 10.

In another preferred embodiment, the polypeptide having dihydrofolate reductase activity comprises an amino acid sequence having at least 70%, such as at least 80%, at least 85%, at least 90%, at least 95%, at least 98% or at least 99%, sequence identity with SEQ ID NO: 12.

In another preferred embodiment, the amino acid sequence of the GTP cyclohydrolase is as shown in SEQ ID NO.: 7.

In another preferred embodiment, the coding sequence of the GTP cyclohydrolase is as shown in SEQ ID NO.: 1.

In another preferred embodiment, the amino acid sequence of the 7,8-dihydroneopterin aldolase is as shown in SEQ ID NO.: 2.

In another preferred embodiment, the coding sequence of the 7,8-dihydroneopterin aldolase is as shown in SEQ ID NO.:8.

In another preferred embodiment, the amino acid sequence of the 2-amino-4-hydroxy-6-hydroxymethyldihydropteridine pyrophosphokinase is as shown in SEQ ID NO.: 3.

In another preferred embodiment, the coding sequence of the 2-amino-4-hydroxy-6-hydroxymethyldihydropteridine pyrophosphokinase is as shown in SEQ ID NO.: 9.

In another preferred embodiment, the amino acid sequence of the dihydropteroate synthase is as shown in SEQ ID NO.: 4.

In another preferred embodiment, the coding sequence of the dihydropteroate synthase is as shown in SEQ ID NO.: 10.

In another preferred embodiment, the amino acid sequence of the dihydrofolate reductase is as shown in SEQ ID NO.: 6.

In another preferred embodiment, the coding sequence of the dihydrofolate reductase is as shown in SEQ ID NO.: 12.

In another preferred embodiment, the engineered strain is obtained by the following method:

(a) Decreasing the expression level and/or activity of the endogenous folC gene in the starting strain, and introducing the exogenous folC gene.

In another preferred embodiment, the method further comprises the step (b) of introducing or upregulating a folate biosynthetic gene in the starting strain.

In another preferred embodiment, the production capacity includes: fermentation yield (productivity).

In the second aspect, it provides a method for preparing a folate, a salt thereof, a precursor thereof, or an intermediate thereof, comprising the steps of:

(i) providing the engineered strain of claim 1;

(ii) cultivating the engineered strain described in the step (i), thereby obtaining a fermentation product containing one or more compounds of the folate, the salt thereof, the precursor thereof, or the intermediate thereof;

(iii) Optionally, the fermentation product obtained in the step (ii) is subjected to separation and purification to further obtain one or more compounds of the folate, the salt thereof, the precursor thereof, or the intermediate thereof;

(iv) Optionally, the product obtained in the steps (ii) or (iii) is subjected to acidic or alkaline conditions to further obtain a different compound of the folate, the salt thereof, the precursor thereof, or the intermediate thereof;

wherein the structural formula of a folate, a salt, a precursor, or an intermediate thereof is as shown in Formula I:

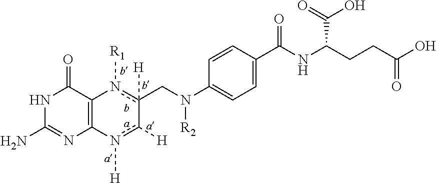

(I); and R₁, R₂, a, a′, b, b′ are defined as above.

In another preferred embodiment, the folate, the salt thereof, the precursor thereof, or the intermediate thereof is folic acid.

In another aspect, it provides a method for preparing a folate a precursor, or an intermediate thereof, comprising the steps of:

(i) providing the engineered strain of claim 1;

(ii) cultivating the engineered strain described in the step (i), thereby obtaining a folate-containing fermentation product;

(iii) Optionally, the fermentation product obtained in the step (ii) is subjected to separation and purification to further obtain a folate, a precursor, or an intermediate thereof.

In another preferred embodiment, the structural formula of a folate, a salt, a precursor, or an intermediate thereof is as shown in Formula I:

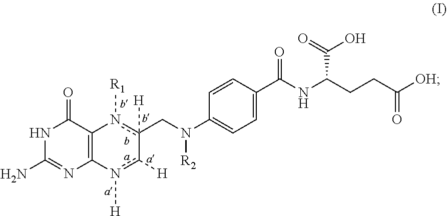

wherein, when a is single bond, a′ is none or when a′ is a single bond, a is none;

when b is a single bond, b′ is none or when b′ is a single bond, b is none;

R1 is selected from the group consisting of: —H, —CH₃ (5-methyl), —CHO (5-formyl), —CH═ or ═CH— (5,10-methenyl), —CH₂— (5,10-methylene), —CH═NH (5-formimino-) and a combination thereof;

R2 is selected from the group consisting of: —H. —CHO (10-formyl), —CH═, ═CH— (5, 10-methenyl), —CH₂— (5,10-methylene) and a combination thereof.

In another preferred embodiment, the culture temperature of the engineered strain is 32-42° C., preferably 34-39° C., more preferably 36-39° C., such as at about 37° C.

In another preferred embodiment, the culture time of the engineered strain is 10-70 h, preferably 24-60 h, more preferably, 36-50 h.

In another preferred embodiment, the pH of the culture of the engineered strain is 6-8, preferably 6.5-7.5, more preferably 6.8-7.2.

In another preferred embodiment, the method further comprises the step of adding para-aminobenzoic acid (PABA) during the cultivation process of step (ii).

In another preferred embodiment, the para-aminobenzoic acid (PABA) is selected from the group consisting of: potassium paraaminobenzoate, sodium para-aminobenzoate, methyl paraaminobenzoate, ethyl para-aminobenzoate, butyl para-aminobenzoate, and a combination thereof.

In another preferred embodiment, further comprising subjecting the product obtained in the steps (i) or (ii) or (iii) to acidic or alkaline conditions to further obtain a derivative compound.

In the third aspect, it provides a method of preparing the engineered strain according to the first aspect of the present invention, comprising the steps of:

(a) decreasing the expression level of the endogenous folC gene in the starting strain, and introducing the exogenous folC gene, thereby obtaining the engineered strain of claim 1.

In another preferred embodiment, the method further comprises the step (b) of introducing or up-regulating a folate synthesis regulatory gene in the starting strain.

In another preferred embodiment, the method comprises the steps of:

(a1) knocking out an endogenous folC gene in a host cell;

(b1) cultivating the host cell; and

the method comprises the steps of:

(a2) providing an expression vector carrying an exogenous folC gene;

(b2) transferring the expression vector into a host cell;

(c2) cultivating the host cell.

In another preferred embodiment, the vector is a plasmid, a cosmid or a nucleic acid fragment.

In the fourth aspect, it provides a use of an engineered strain according to the first aspect of the present invention, which is used as an engineered strain for fermentative production of a folate, a salt, a precursor or an intermediate thereof.

In the fifth aspect, it provides a genetically engineered microorganism, preferably bacterium or yeast, which has been modified to i) have a decreased expression level of the endogenous gene encoding a polypeptide having both dihydrofolate synthase activity and folylpolyglutamate synthetase activity compared to an otherwise identical microorganism (reference microorganism), and ii) express a heterologous polypeptide having only dihydrofolate synthase activity.

In another preferred embodiment, the expression level of the endogenous gene is decreased by at least 50%, such as by at least 60%, at least 70%, at least 80%, at least 90% or at least 100% compared to the otherwise identical microorganism.

In another preferred embodiment, the endogenous gene encoding a polypeptide having both dihydrofolate synthase activity and folylpolyglutamate synthetase activity has been inactivated.

In another preferred embodiment, the endogenous gene encoding a polypeptide having both dihydrofolate synthase activity and folylpolyglutamate synthetase activity has been inactivated by deletion of part of or the entire gene sequence.

In another preferred embodiment, the endogenous gene encoding a polypeptide having both dihydrofolate synthase activity and folylpolyglutamate synthetase activity is the gene folC.

In another preferred embodiment, the endogenous gene encoding a polypeptide having both dihydrofolate synthase activity and folylpolyglutamate synthetase activity is the endogenous gene folC.

In another preferred embodiment, the endogenous gene encoding a polypeptide having both dihydrofolate synthase activity and folylpolyglutamate synthetase activity comprises a nucleic acid sequence having at least 70%, such as at least 80%, at least 85%, at least 90%, at least 95%, at least 98% or at least 99%, sequence identity with the nucleic acid sequence set forth in SEQ ID NO: 5.

In another preferred embodiment, the polypeptide having both dihydrofolate synthase activity and folylpolyglutamate synthetase activity encoded by the endogenous gene comprises an amino acid which has at least 70%, such as at least 80, at least 85%, at least 90%, at least 95%, at least 98% or at least 99%, sequence identity with the amino acid sequence set forth in SEQ ID NO: 11.

In another preferred embodiment, the heterologous polypeptide having only dihydrofolate synthase activity is derived from a bacterium or fungus, preferably selected from Lactobacillus reuteri and Ashbya gossypii.

In another preferred embodiment, the heterologous polypeptide having only dihydrofolate synthase activity comprises an amino acid sequence having at least 70%, such as at least 80%, at least 85%, at least 90%, at least 95%, at least 98% or at least 99%, sequence identity with SEQ ID NO: 22 or 23.

In another preferred embodiment, the genetically engineered microorganism has been further modified to have a significantly improved production capacity of a folate, a precursor or an intermediate thereof compared to an otherwise identical microorganism (reference microorganism).

In another preferred embodiment, the production capacity of a folate, a precursor or an intermediate thereof is increased by at least 50%, such as at least 100%, at least 200%, at least 500%, at least 1000%, at least 2000%, at least 5000%, at least 10000%, at least 20000% or at least 50000%, compared to an otherwise identical microorganism.

In another preferred embodiment, the genetically engineered microorganism has been further modified to have an increased expression level of at least one gene (such as at least two, at least three, at least four, or at least five genes) encoding an enzyme involved in the biosynthesis of folic acid compared to an otherwise identical microorganism.

In another preferred embodiment, the expression level of at least one gene (such as at least two, three four, or five genes) encoding an enzyme involved in the biosynthesis of folic acid is increased by at least 50%, such as by at least 100%, at least 200%, at least 500%, at least 1000%, at least 2000%, at least 5000%, at least 10000%, at least 20000% or at least 50000%, compared to an otherwise identical microorganism.

In another preferred embodiment, the at least one gene encoding an enzyme involved in the biosynthesis of folic acid is selected from the group consisting of folE/mtrA, folB, folK, folP/sul, and folA/dfrA.

In another preferred embodiment, the enzyme involved in the biosynthesis of folic acid is selected from selected from the group consisting of: a polypeptide having GTP cyclohydrolase activity, a polypeptide having 7,8-dihydroneopterin aldolase activity, a polypeptide having 2-amino-4-hydroxy-6-hydroxymethyldihydropteridine pyrophosphokinase activity, a polypeptide having dihydropteroate synthase activity, and a polypeptide having dihydrofolate reductase activity.

In another preferred embodiment, the at least one gene encoding an enzyme involved in the biosynthesis of folic acid is heterologous to the genetically engineered microorganism.

In another preferred embodiment, the at least one gene encoding an enzyme involved in the biosynthesis of folic acid is derived from a bacterium or fungus, preferably selected from the genus Bacillus, Lactococcus and Ashbya.

In another preferred embodiment, the at least one gene encoding an enzyme involved in the biosynthesis of folic acid is derived from a bacterium or fungus selected from Bacillus subtiltis, Lactobacillus lactis and Ashbya gossypii.

In another preferred embodiment, the genetically engineered microorganism has been further modified to have an increased expression level of at least one enzyme (such as at least two, at least three, at least four, or at least five enzymes) involved in the biosynthesis of folic acid compared to an otherwise identical microorganism.

In another preferred embodiment, said at least one enzyme involved in the biosynthesis of folic acid is selected from the group consisting of: a polypeptide having GTP cyclohydrolase activity, a polypeptide having 7,8-dihydroneopterin aldolase activity, a polypeptide having 2-amino-4-hydroxy-6-hydroxymethyldihydropteridine pyrophosphokinase activity, a polypeptide having dihydropteroate synthase activity, and a polypeptide having dihydrofolate reductase activity.

In another preferred embodiment, the at least one enzyme involved in the biosynthesis of folic acid is heterologous to the genetically engineered microorganism.

In another preferred embodiment, the at least one enzyme involved in the biosynthesis of folic acid is derived from a bacterium or fungus, preferably selected from the genus Bacillus, Lactococcus, Shewanella, Vibrio and Ashbya.

In another preferred embodiment, the at least one enzyme involved in the biosynthesis of folic acid is derived from Bacillus subtiltis, Lactobacillus lactis, Shewanella violacea, Vibrio natriegens or Ashbya gossypii.

In another preferred embodiment, the polypeptide having GTP cyclohydrolase activity comprises an amino acid sequence having at least 70%, such as at least 80%, at least 85%, at least 90%, at least 95%, at least 98% or at least 99%, sequence identity with SEQ ID NO: 7.

In another preferred embodiment, the polypeptide having 7,8-dihydroneopterin aldolase activity comprises an amino acid sequence having at least 70%, such as at least 80%, at least 85%, at least 90%, at least 95%, at least 98% or at least 99%, sequence identity with SEQ ID NO: 8.

In another preferred embodiment, the polypeptide having 2-amino-4-hydroxy-6-hydroxymethyl-dihydropteridine pyrophosphokinase activity comprises an amino acid sequence having at least 70%, such as at least 80%, at least 85%, at least 90%, at least 95%, at least 98% or at least 99%, sequence identity with SEQ ID NO: 9.

In another preferred embodiment, the polypeptide having dihydropteroate synthase activity comprises an amino acid sequence having at least 70%, such as at least 80%, at least 85%, at least 90%, at least 95%, at least 98% or at least 99%, sequence identity with SEQ ID NO: 10.

In another preferred embodiment, the polypeptide having dihydrofolate reductase activity comprises an amino acid sequence having at least 70%, such as at least 80%, at least 85%, at least 90%, at least 95%, at least 98% or at least 99%, sequence identity with SEQ ID NO: 12.

In another preferred embodiment, the genetically engineered microorganism is a bacterium.

In another preferred embodiment, the genetically engineered microorganism is a bacterium of the genus Bacillus.

In another preferred embodiment, the genetically engineered microorganism is a bacterium of species Bacillus subtiltis.

In the sixth aspect, it provides a method for preparing folate or a salt, precursor or intermediate thereof, comprising i) cultivating a genetically engineered microorganism according to the fifth aspect of the present invention in a culture medium under suitable culture conditions to obtain a fermentation product containing said folic acid, precursor or intermediate thereof; and ii) optionally, separating and/or purifying said folic acid, precursor or intermediate thereof.

In another preferred embodiment, step i) is carried out at a culture temperature in a range from 32 to 42° C., preferably in a range from 34 to 39° C., more preferably in a range from 36 to 39° C., such as at about 37° C.

In another preferred embodiment, step i) is carried out for a period in the range from 10 to 70 h, preferably in a range from 24 to 60 h, more preferably in a range from 36 to 50 h.

In another preferred embodiment, wherein step i) is carried out at a pH in the range of 6 to 8, preferably in a range of 6.5 to 7.5, more preferably in a range from 6.8 to 7.2.

In another preferred embodiment, the folate or salt, precursor or intermediate thereof is a compound of Formula I:

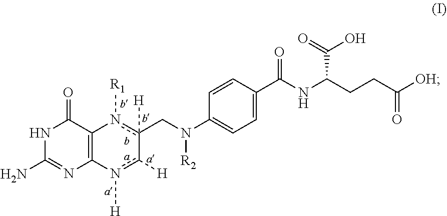

wherein, when a is single bond, a′ is none or when a′ is a single bond, a is none;

when b is a single bond, b′ is none or when b′ is a single bond, b is none;
R1 is selected from the group consisting of: —H, —CH₃ (5-methyl), —CHO (5-formyl), —CH═ or ═CH— (5,10-methenyl), —CH₂— (5,10-methylene), and —CH═NH (5-formimino-);
   R2 is selected from the group consisting of: —H, —CHO (10-formyl), —CH═, ═CH— (5,10-methenyl), and —CH₂— (5,10-methylene).
   In another preferred embodiment, further comprising the step of adding para-aminobenzoic acid (PABA) during the cultivation step (i).
   In another preferred embodiment, the para-aminobenzoic acid (PABA) is selected from the group consisting of: potassium paraaminobenzoate, sodium para-aminobenzoate, methyl paraaminobenzoate, ethyl para-aminobenzoate, butyl para-aminobenzoate, and a combination thereof.
   In another preferred embodiment, further comprising subjecting the product obtained in the steps (i) or (ii) to acidic or alkaline conditions to further obtain a derivative compound.
   In another preferred embodiment, comprising the steps of (a) decreasing the expression level of the endogenous gene encoding a polypeptide having both dihydrofolate synthase activity and folylpolyglutamate synthetase activity compared to an otherwise identical microorganism (reference microorganism), and b) expressing a heterologous polypeptide having only dihydrofolate synthase activity.
   In another preferred embodiment, comprising the steps of aa) inactivating, such as by deleting part of or the entire gene sequence, the endogenous gene encoding a polypeptide having both dihydrofolate synthase activity and folylpolyglutamate synthetase activity in said microorganism; and/or bb) introducing into said microorganism an exogenous nucleic acid molecule comprising a nucleic acid sequence encoding a heterologous polypeptide having only dihydrofolate synthase activity.

It should be understood that, within the scope of the present invention, each technical feature of the present invention described above and in the following (as examples) may be combined with each other to form a new or preferred technical solution, which is not listed here due to space limitations.

### DESCRIPTION OF FIGURES

FIG. 1 shows the core structure of folates. In natural folates, the pterin ring exists in tetrahydro form (as shown) or in 7,8-dihydro form. The ring is fully oxidized in chemically produced folic acid. Folates usually have a γ-linked polyglutamyl tail of up to about eight residues attached to the first glutamate. One-carbon unit (formyl, methyl, etc.) can be coupled to the N5 and/or N10 positions resulting in synthesis of 5-formyl folates, 10-formyl folates or 5-methyl folates.

FIG. 2 shows schematic representation of an example of a folic acid operon consisting of L. lactis genes.

FIG. 3 shows schematic representation of an example of a folic acid operon consisting of A. gossypii genes.

FIG. 4 shows schematic representation of an example of a folic acid operon consisting of B. subtilis genes.

FIG. 5 shows schematic presentation of the FolC disruption cassettes with tetracycline resistance gene (TetR), heterologous folC2-LR or folC2-AG gene under P_{veg} promoter and flanking homology ends for native folC target gene disruption. The position of the primers used for PCR amplification of the DNA disruption cassette are denoted as lines.

FIG. 6 shows chromatogram of 10-formyl folic acid standard. Black: UV signal, red: MS/MS signal.

FIG. 7 shows SRM fragments originating from m/z 470 at CE 20 V.

FIG. 8 shows chromatogram of 5-formyl-THF standard. Black: UV signal, red: MS/MS signal.

FIG. 9 shows SRM fragments originating from m/z 474 at CE 20 V.

FIG. 10 shows chromatogram of 5-methyl-THF standard. Black: UV signal, red: MS/MS signal.

FIG. 11 shows SRM fragments originating from m/z 460 at CE 20 V and chromatogram of fermentation broth sample. Black: UV signal, red: MS scan signal.

FIG. 12 shows SRM fragments originating from m/z 472 at CE 20 V. Identity of new peak at RT=10 min is confirmed as 10-dihydro-formyl folic acid.

FIG. 13 shows chromatogram of fermentation broth sample. Black: UV signal, red: MS scan signal.

FIG. 14 shows schematic representation of oxidation of 10-formyldihydrofolic acid to 10-formylfolic acid in the presence of oxygen, schematic representation of oxidation of 10-formyldihydrofolic acid to 10-formylfolic acid in the presence of hydrogen peroxide and schematic representation of oxidation of 10-formyldihydrofolic acid to 10-formylfolic acid in the presence of sodium periodate.

FIG. 15 shows schematic representation of deformylation of 10-formylfolic acid to folic acid in acidic medium.

FIG. 16 shows schematic representation of deformylation of 10-formylfolic acid to folic acid in alkaline medium.

FIG. 17 shows Folates production bioprocess profile. Folates (mg/L): full stars; Glucose concentration (g/L): empty squares; Acetoin concentration (g/L): full squares; PABA concentration (mg/L): empty circles; PABA feed (mg/L): vertical bars; Optical density: full circles.

FIG. 18 shows total folate production titers of B. subtilis strain w.t. 168, strain VBB38, strain FL21 and FL23 at the shaker 5 ml scale experiments.

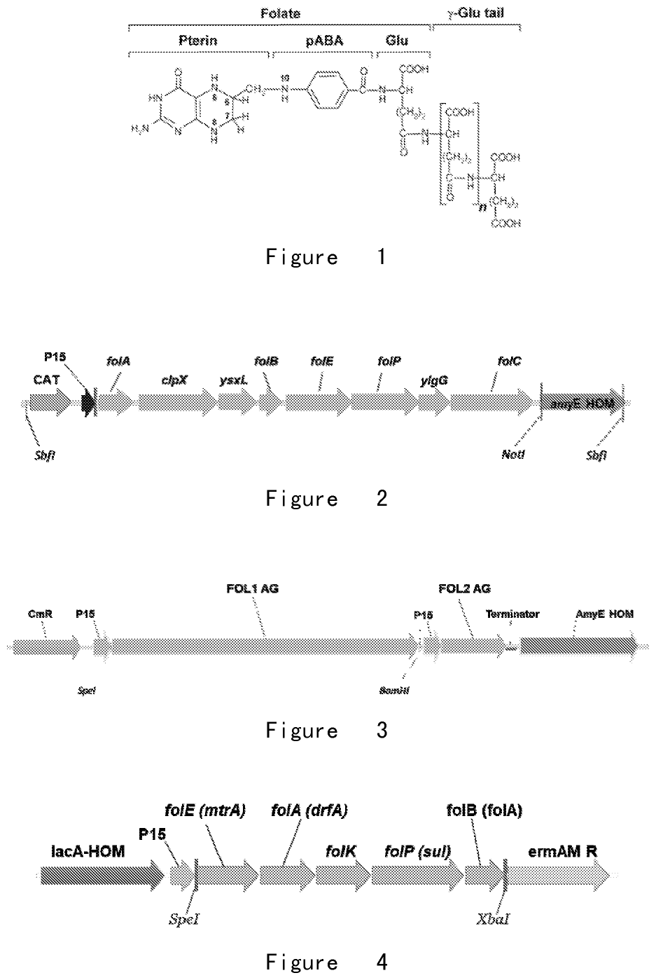

### DETAILED DESCRIPTION

After extensive and intensive research and a lot of screening, the inventors have unexpectedly discovered that if the expression level of the endogenous folC gene is reduced in the starting strain, and the exogenous folC gene is simultaneously introduced, and only one glutamate is added on the biosynthesized folate, and the production capacity of a folate, a salt, a precursor, or an intermediate thereof is significantly increased. In addition, the present inventors have also found that introduction or up-regulation of folate biosynthetic genes (such as, folE/mtrA, folB, folK, folP/sul, folA/dfrA) in the starting strain can also significantly increase the production capacity of a folate, a salt, a precursor, or an intermediate thereof. The inventors have also unexpectedly discovered that the addition of para-aminobenzoic acid (PABA) during the cultivation of the strain, obtained as described above, can significantly further increase the production capacity of a folate, a salt, a precursor, or an intermediate thereof. On the basis of this, the inventors completed the present invention.

“Heterologous” as used herein means that a polypeptide is normally not found in or made (i.e. expressed) by the host organism, but derived from a different species.

“Inactivating” as used herein that the gene in question no longer expresses a functional protein. It is possible that the modified DNA region is unable to naturally express the gene due to the deletion of a part of or the entire gene sequence, the shifting of the reading frame of the gene, the introduction of missense/nonsense mutation(s), or the modification of an adjacent region of the gene, including sequences controlling gene expression, such as a promoter, enhancer, attenuator, ribosome-binding site, etc. Preferably, a gene of interest is inactivated by deletion of a part of or the entire gene sequence, such as by gene replacement.

The presence or absence of a gene on the chromosome of a bacterium can be detected by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the protein encoded by the gene can be measured by well-known methods, including SDS-PAGE followed by an immunoblotting assay (Western blotting analysis), and the like.

In the present invention, the terms “genetically engineered strain” and “the genetically engineered microorganism” can be used interchangeably.

Starting Strain

As used herein, the terms “the starting strain of the present invention” or “the starting microorganism of the present invention” can be used interchangeably and refer to any bacterium or fungus encoding in its genome a polypeptide having both dihydrofolate synthase activity and folylpolyglutamate synthetase activity such as any Bacillus species e.g. Bacillus subtilis.

In a preferred embodiment, the starting strain is obtained or purchased from the Russian National Collection of Industrial Microorganisms at the Institute of Genetics and Selection of Industrial Microorganisms, numbered VKPM B-2116, alternative names VNIIGenetika-304 or VBB38.

The physiological and biochemical properties of the starting strains of the present invention are: deregulation of the biosynthesis of riboflavin, deregulation of the biosynthesis of purine bases, capacity to grow in the presence of 8-azaguanine capacity to grow in the presence of roseoflavin.

It should be understood that the starting strain not only includes the strain with the numbering of VKPM B-2116. The strain also includes its derived strains.

Folate, the Salt, the Precursor or the Intermediate Thereof

In the present invention, folate, the salt, the precursor or the intermediate thereof is as shown in formula I:

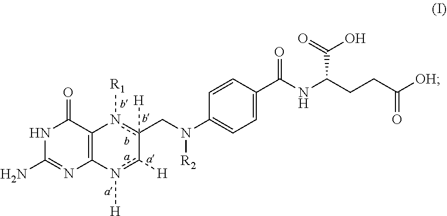

wherein, when a is single bond, a′ is none or when a′ is a single bond, a is none;

when b is a single bond, b′ is none or when b′ is a single bond, b is none;

R1 is selected from the group consisting of: —H, —CH₃ (5-methyl), —CHO (5-formyl), —CH═ or ═CH— (5,10-methenyl), —CH₂— (5,10-methylene), —CH═NH (5-formimino-), and a combination thereof;

R2 is selected from the group consisting of: —H. —CHO (10-formyl), —CH═, ═CH— (5, 10-methenyl), —CH₂— (5,10-methylene) and a combination thereof.

Folate is an important vitamin from the group of B vitamins, which is widely used for food and animal feed fortification and production of dietary supplements. Folate is often used as a supplement by women during pregnancy to reduce the risk of neural tube defects in the baby. Long-term supplementation is also associated with small reductions in the risk of stroke and cardiovascular disease.

“Folate” is the term used to name the many forms of the vitamin-namely folic acid and its congeners, including tetrahydrofolic acid (the activated form of the vitamin), methyltetrahydrofolate (the primary form found in the serum), methenyltetrahydrofolate, folinic acid, and folacin.

The traditional folate production is based on chemical synthesis. The major three components, 2,4,5-triamino-6-hydroxypyrimidine, 1,1,3-trichloroacetone and N-(4-aminobenzoyl)-L-glutamic acid are condensed to produce pteroic acid mono glutamate via acid precipitation and alkali refining. This chemical production process of folic acid has disadvantages, such as low yield, generation of huge amounts of waste water, leading to serious environmental pollution.

The inventors have found that by genetically engineering a starting strain, the production capacity of a folate, a salt, a precursor or an intermediate thereof in the strain can be significantly improved.

The “production capacity of the folate, the salt, the precursor or the intermediate thereof” of the present invention refers to the production capacity of the folate compounds, the salts, the precursors or the intermediates thereof, that is, which is equivalent to the “industrial production grade”, “industrial potential”, “industrial production capacity”, “production capacity” of the precursor or the intermediate thereof, which can be used interchangeably, referring to the fermentation yield is at least 0.01 g/L, preferably at least 0.05-0.1 g/L; more preferably at least 0.5-1 g/L according to the total volume of the fermentation broth, and any integer and non-integer values in this range, which are not mentioned here.

The experiment of the present invention shows that the genetically engineered strain of the present invention (such as Bacillus subtilis) significantly increases the synthesis ability of folate, the salt, the precursor, or the intermediate thereof, and yield can reach 333 mg/L in shake flask experiment. In the wild-type strain (such as Bacillus subtilis), the synthesis ability of folate, the precursor, or the intermediate thereof is very low, and the yield only can reach 0.31 mg/L. This is very unexpected.

folC Gene

In some bacteria, such as Bacillus subtilis, the addition of L-glutamate to dihydropteroate (dihydrofolate synthetase (DHFS) activity, EC 6.3.2.12) and the subsequent additions of L-glutamate to tetrahydrofolate through gamma carboxyl groups (folylpolyglutamate synthetase (FPGS) activity, EC 6.3.2.17) are catalyzed by the same enzyme, FolC. In contrast, in eukaryotes and some other bacteria DHFS and FPGS enzymatic activities are encoded in different genes. B. subtilis, as many other bacteria, adds gamma-linked poly-glutamate tails to folates in order to increase solubility and prevent the loss of this essential cofactor into the environment. Thus, the Bacillus subtilis FolC possesses folyl-poly-glutamate synthetase (FPGS) activity which catalyzes the polyglutamylation of folates through their gamma-carboxyl groups in addition to its role as dihydrofolate synthase in the de novo folate biosynthetic pathway. The folate polyanions cannot be exported out of cells, resulting in enhanced intracellular retention (Sybesma et al., 2003c). In addition, the products of the FPGS enzyme, folyl-polyglutamates, are strong inhibitors of the folate biosynthetic enzymes (McGuire and Bertino, 1981). Therefore, in order to increase the production of folates, we have abolished the polyglutamylation of folates by knocking-out the native folC gene and replaced it with a heterologous folC gene encoding only for the essential dihydrofolate synthetase (DHFS) activity, resulting in the addition of only one essential glutamate moiety. Homologs of FolC with only the dihydrofolate synthetase (DHFS) and without folylpolyglutamate (FGPS) synthetase activity can be found in many bacteria species like Lactobacillus reuteri and many eukaryotic organisms like Ashbya gossypii.

Folate Biosynthetic Gene

In the present invention, the folate biosynthetic genes include folE/mtrA, folB, folK, folP/sul, and folA/dfrA.

The folate molecule contains one pterin moiety, originating from guanosine triphosphate (GTP), bound to para aminobenzoic acid (pABA) and at least one molecule of glutamic acid. Thus, de novo biosynthesis of folate requires three precursors: GTP, pABA and glutamic acid.

Folate biosynthesis proceeds via the conversion of GTP to the 6-hydroxymethyl-7,8-dihydropterin pyrophosphate (DHPPP) in four consecutive steps. The first step is catalyzed by GTP cyclohydrolase I (EC 3.5.4.16) (gene folE/mtrA) and involves an extensive transformation of GTP, to form a pterin ring structure. Following dephosphorylation, the pterin molecule undergoes aldolase (EC 4.1.2.25) (gene folB) and pyrophosphokinase reactions (EC 2.7.6.3) (gene folK), which produce the activated pyrophosphorylated DHPPP. Following the first condensation of para-aminobenzoic acid (pABA) with DHPPP catalyzed by dihydropteroate synthase (EC 2.5.1.15) (gene folP/sul) to produce dihydropteroate. The second condensation is reaction of glutamate with dihydropteroate to form dihydrofolate by dihydrofolate synthase (DHFS) (EC 6.3.2.12) (gene folC). Then, DHF is reduced by DHF reductase-DHFR (EC 1.5.1.3) (gene folA/dfrA) to the biologically active cofactor tetrahydrofolate (THF).

In the present invention, information on the folate biosynthetic gene is shown in Table 1.

TABLE 1
Folate biosynthetic genes

| | | | | Nucleotide |
|---|---|---|---|---|
| | | | | sequence |
| | Microo | | | *optimised |
| Gene name | organism | NCBI | | sequence for |
| synonyme and | (gene | access | Protein | Bacillus subtil |
| enzymatic activity | source) | number | sequence | codon usage |
| folC2-AG/FOL3 | Ashbya | NP_984550 | >NP_984550.1 | atggagttaggcttaggccgcatc |
| (dihydrofolate | gossypii | | AEL310Cp | acacaagtgctgagacaattacata |
| synthetase) | | | [Eremothecium | gccctcatgaaagaatgcgtgtctt |
| | | | gossypii ATCC 10895] | acatgttgcaggaacaaatggcaa |
| | | | MELGLGRITQVLRQL | aggaagcgtctgtgcgtatttagcg |
| | | | HSPHERMRVLHVAG | gctgttttaagagcgggcggagaa |
| | | | TNGKGSVCAYLAAV | agagttggcagatttacaagccctc |
| | | | LRAGGERVGRFTSPH | acttagttcatccgcgcgatgctat |
| | | | LVHPRDAITVDGEVI | cacagtcgacggcgaagttattgg |
| | | | GAATYAALKAEVVA | agcggcgacatatgctgcacttaa |
| | | | AGTCTEFEAQTAVAL | agctgaagtcgttgcggcaggcac |
| | | | THFARLECTWCVVE | atgcacggagtttgaagcacaaac |
| | | | VGVGGRLDATNVVP | ggcggttgcgcttacgcattttgca |
| | | | GGRKLCAITKVGLDH | agacttgaatgcacatggtgtgtcg |
| | | | QALLGGTLAVVARE | tcgaagtgggcgtcggcggcaga |
| | | | KAGIVVPGVRFVAV | ttagacgctacaaatgtcgtccctg |
| | | | DGTNAPSVLAEVRA | gcggacgcaaactgtgtgcaatta |
| | | | AAAKVGAEVHETGG | caaaggttggattagatcatcaggc |
| | | | APVCTVSWGAVAAS | gttacttggcggaacactggctgtt |
| | | | ALPLAGAYQVQNAG | gttgcaagagagaaggccggcatt |
| | | | VALALLDHLQQLGEI | gtggttccgggagtgcgctttgtcg |
| | | | SVSHAALERGLKAVE | ctgtcgacggcacgaacgcacctt |
| | | | WPGRLQQVEYDLGG | cagttctggcggaggttcgggcgg |
| | | | VHVPLLFDGAHNPC | ctgcagcgaaagttggcgcagag |
| | | | AAEELARFLNERYRG | gtccatgagacaggaggcgcgcc |
| | | | PGGSPLIYVLAVTCG | ggtttgcacagtcagctggggtgc |
| | | | KEIDALLAPLLKPHD | ggttgctgcaagcgcacttccgtta |
| | | | RVFATSFGAVESMP | gcgggagcttaccaggtacaaaac |
| | | | WVAAMASEDVAAA | gcgggcgttgcacttgcactgcttg |
| | | | ARRYTAHVSAVADP | atcatcttcaacaactgggagagat |
| | | | LDALRAAAAARGDA | ctcagtcagccatgcagcactgga |
| | | | NLVVCGSLYLVGELL | aagaggactgaaagcagtcgaat |
| | | | RREH | ggcctggcagacttcaacaagttg |
| | | | (SEQ ID NO.: 73) | agtatgaccttggaggcgtccatgt |
| | | | | cccgctgttatttgacggagcacac |
| | | | | aatccgtgtgcagcggaagagctt |
| | | | | gcaagattcttaaacgagagatac |
| | | | | cgcggaccgggaggatctccgct |
| | | | | gatctatgtgctggctgtcacgtgt |
| | | | | ggcaaagagatcgacgcacttctt |
| | | | | gcacctcttctgaaaccgcacgata |
| | | | | gagtcttcgcaaccagctttggcgc |
| | | | | ggttgagtctatgccgtgggtcgca |
| | | | | gcgatggcaagcgaggatgtcgc |
| | | | | agcggcggcgagacgctacacag |
| | | | | cccacgtttcagcggttgcggacc |
| | | | | cgctggacgcgttacgcgccgca |
| | | | | gcggcagcacgcggcgatgctaa |
| | | | | tctggtcgtctgcggatcattatatc |
| | | | | ttgtcggcgaacttctgcgccgcg |
| | | | | aacattaa |
| | | | | (SEQ ID NO.: 74) |
| folC2-LR | Lactobacillus | BAG_25726 | >BAG25726.1 | atgagaacatacgaacaaattaatg |
| (dihydrofolate | reuteri | | folylpoly glutamate | caggatttaatcgccagatgctgg |
| synthetase) | | | synthase [Lactobacillus | gcggccagagagacagagtcaag |
| | | | reuteri JCM 1112] | ttccttagacgcatccttacgagact |
| | | | MRTYEQINAGFNRQ | tggaaaccctgatcagcgctttaaa |
| | | | MLGGQRDRVKFLRRI | attattcatatcgcgggaacgaacg |
| | | | LTRLGNPDQRFKIIHI | gcaaaggatcaacaggcactatgt |
| | | | AGTNGKGSTGTMLE | tagaacagggccttcagaatgcgg |
| | | | QGLQNAGYRVGYFS | gataccgcgtcggctactttagctc |
| | | | SPALVDDREQIKVND | tcctgcgctggttgatgatcgcgaa |
| | | | HLISKKDFAMTYQKI | caaattaaagtcaatgatcaccttat |
| | | | TEHLPADLLPDDITIF | cagcaagaaagattttgcgatgac |
| | | | EWWTLIMLQYFADQ | ctatcagaaaattacggagcatctg |
| | | | KVDWAVIECGLGGQ | cctgctgaccttctgcctgatgatat |
| | | | DDATNIISAPFISVITH | tacaatctttgagtggtggacgttaa |
| | | | IALDHTRILGPTIAKI | tcatgcttcaatactttgcggatcaa |
| | | | AQAKAGIIKTGTKQV | aaggttgactgggcggtgattgaat |
| | | | FLAPHQEKDALTIIRE | gtggtcttggcggccaagacgatg |
| | | | KAQQQKVGLTQADA | cgacaaacatcatctcagcgccgtt |
| | | | QSIVDGKAILKVNHK | catttcagtcattacccatatcgctct |
| | | | IYKVPFNLLGTFQSE | tgaccacacccgtatcctgggccc |
| | | | NLGTVVSVFNFLYQR | tacaattgcgaagattgcgcaagct |
| | | | RLVTSWQPLLSTLAT | aaggcaggcattataaagacagg |
| | | | VKIAGRMQKIADHPP | gactaaacaggttttcctggcacca |
| | | | IILDGAHNPDAAKQL | catcaagagaaggatgcgttaaca |
| | | | TKTISKLPHNKVIMV | atcattcgcgaaaaagcgcaacag |
| | | | LGFLADKNISQMVKI | caaaaggtcggactgacgcaggc |
| | | | YQQMADEIIITTPDHP | agatgcacagagcattgtggacgg |
| | | | TRALDASALKSVLPQ | aaaagctattttaaaagtgaatcac |
| | | | AIIANNPRQGLVVAK | aagatttacaaggtcccttttaatct |
| | | | KIAEPNDLIIVTGSFY | gctgggcacatttcagtcagaaaa |
| | | | TIKDIEANLDEK | cctgggaacggttgttagcgtcttt |
| | | | (SEQ ID NO.: 75) | aactttctgtatcagcgccgtcttgt |
| | | | | cacgtcatggcaaccgttacttagc |
| | | | | acactggcaacagttaaaattgca |
| | | | | ggaagaatgcaaaaaatcgcggat |
| | | | | catcctccgatcattcttgatggcg |
| | | | | cacataatccggatgctgcaaagc |
| | | | | agcttacaaagacaattagcaaact |
| | | | | cccacataataaagtcataatggtg |
| | | | | ttaggcttccttgctgacaaaaacat |
| | | | | ttcacagatggtcaagatttaccaa |
| | | | | cagatggcggatgaaattatcatta |
| | | | | caacgcctgaccatcctacaagag |
| | | | | cgctggacgcctcagcccttaaat |
| | | | | cagtcttaccgcaagcaattattgc |
| | | | | gaataatcctcgtcagggactggtt |
| | | | | gttgctaagaaaattgcagagccg |
| | | | | aacgatcttatcatcgtcacgggca |
| | | | | gcttctacacaatcaaggatattga |
| | | | | ggcaaatttagatgagaaataa |
| | | | | (SEQ ID NO.: 76) |
| folE/mtrA | Bacillus | NP_390159 | >NP_390159.1 GTP | atgaaagaagtcaataaagaacaa |
| (GTP cyclohydrolase) | subtilis | | cyclohydrolase I | attgaacaggcagtgagacagatt |
| | | | [Bacillus subtilis subsp. | cttgaagcaattggagaagatccg |
| | | | subtilis str. 168] | aacagagagggcttacttgataca |
| | | | MKEVNKEQIEQAVR | ccgaaaagagttgctaaaatgtatg |
| | | | QILEAIGEDPNREGLL | cggaagtcttttcaggcttaaatga |
| | | | DTPKRVAKMYAEVF | agatccgaaagagcattttcagac |
| | | | SGLNEDPKEHFQTIF | aattttcggagaaaaccatgaaga |
| | | | GENHEELVLVKDIAF | gctggtccttgtgaaagatattgcg |
| | | | HSMCEHHLVPFYGK | tttcactcaatgtgcgaacatcacct |
| | | | AHVAYIPRGGKVTGL | ggtgccgttttacggcaaggcaca |
| | | | SKLARAVEAVAKRP | cgttgcgtatattcctagaggcgga |
| | | | QLQERITSTIAESIVET | aaagttacaggcttgtcaaaattag |
| | | | LDPHGVMVVVEAEH | cacgcgcagttgaagctgttgcaa |
| | | | MCMTMRGVRKPGA | aaagaccgcaattacaggaacgc |
| | | | KTVTSAVRGVFKDD | attacatctacaattgcggaatcaat |
| | | | AAARAEVLEHIKRQD | tgtcgagacattagaccctcatggc |
| | | | (SEQ ID NO.: 77) | gttatggttgtcgttgaagctgaac |
| | | | | acatgtgcatgacaatgcgcggcg |
| | | | | tcagaaaacctggcgcaaaaaca |
| | | | | gtcacatcagcagtcagaggcgtg |
| | | | | tttaaagatgatgcggcagctcgtg |
| | | | | cggaagtcctggaacatattaaac |
| | | | | gccaggactga (SEQ ID |
| | | | | NO.: 78) |
| folB | Bacillus | NP_387959 | >NP_387959.1 | Atggataaagtttatgtggaagga |
| (7,8-dihydroneopterin | subtilis | | dihydroneopterin | atggaattttatggctatcatggcgt |
| aldolase) | | | aldolase [Bacillus | cttcacagaagagaacaaattggg |
| | | | subtilis subsp. subtilis | acaacgcttcaaagtagatctgaca |
| | | | str. 168] | gcagaactggatttatcaaaagca |
| | | | MDKVYVEGMEFYGY | ggacaaacagacgaccttgaaca |
| | | | HGVFTEENKLGQRFK | gacaattaactatgcagagctttac |
| | | | VDLTAELDLSKAGQT | catgtctgtaaagacattgtcgaag |
| | | | DDLEQTINYAELYHV | gcgagccggtcaaattggtagaga |
| | | | CKDIVEGEPVKLVET | cccttgctgagcggatagctggca |
| | | | LAERIAGTVLGKFQP | cagttttaggtaaatttcagccggtt |
| | | | VQQCTVKVIKPDPPIP | caacaatgtacggtgaaagttatta |
| | | | GHYKSVAIEITRKKS | aaccagatccgccgattcctggcc |
| | | | (SEQ ID NO.: 79) | actataaatcagtagcaattgaaatt |
| | | | | acgagaaaaaagtcataa |
| | | | | (SEQ ID NO.: 80) |
| folK | Bacillus | NP_387960 | >NP_387960.1 | Atgaacaacattgcgtacattgcg |
| (2-amino-4-hydroxy-6- | subtilis | | 7,8-dihydro-6-hydroxy | cttggctctaatattggagatagag |
| hydroxymethyl- | | | methylpterin | aaacgtatctgcgccaggccgttg |
| dihydropteridine | | | pyrophosphokinase | cgttactgcatcaacatgctgcggt |
| pyrophosphokinase) | | | [Bacillus subtilis subsp. | cacagttacaaaagtcagctcaatt |
| | | | subtilis str. 168] | tatgaaacagatccggtcggctatg |
| | | | MNNIAYIALGSNIGD | aagaccaagcccagtttttaaatat |
| | | | RETYLRQAVALLHQ | ggcggttgaaattaaaacaagcct |
| | | | HAAVTVTKVSSIYET | gaatccgtttgaacttctggaactg |
| | | | DPVGYEDQAQFLNM | acacagcaaatcgaaaacgaactg |
| | | | AVEIKTSLNPFELLEL | ggccgcacacgcgaagttagatg |
| | | | TQQIENELGRTREVR | gggcccgagaacagcggatttag |
| | | | WGPRTADLDILLFNR | acattctgctgtttaacagagaaaa |
| | | | ENIETEQLIVPHPRMY | cattgaaacagagcagttaattgtc |
| | | | ERLFVLAPLAEICQQ | ccgcatcctcgcatgtatgaacgc |
| | | | VEKEATSAETDQEGV | ctgtttgttcttgcgccgcttgcgga |
| | | | RVWKQKSGVDEFVH | aatttgccagcaggtcgagaaaga |
| | | | SES | agcgacaagcgcggaaacggatc |
| | | | (SEQ ID NO.: 81) | aagaaggagttagagtttggaaac |
| | | | | aaaaatcaggcgttgacgaatttgt |
| | | | | acatagcgaaagctga |
| | | | | (SEQ ID NO.: 82) |
| folP/sul | Bacillus | NP_387958 | >NP_387958.1 | Atggcgcagcacacaatagatca |
| (dihydropteroate | subtilis | | dihydropteroate | aacacaagtcattcatacgaaacc |
| synthase) | | | synthase [Bacillus | gagcgcgctttcatataaagaaaa |
| | | | subtilis subsp. subtilis | aacactggtcatgggcattcttaac |
| | | | str. 168] | gttacacctgattcttttagcgatgg |
| | | | MAQHTIDQTQVIHTK | tggaaaatatgacagcttggacaa |
| | | | PSALSYKEKTLVMGI | ggcgcttctgcatgccaaagaaat |
| | | | LNVTPDSFSDGGKYD | gatcgacgacggcgcgcacattat |
| | | | SLDKALLHAKEMIDD | tgacataggaggcgagagcacaa |
| | | | GAHIIDIGGESTRPGA | gaccgggagctgaatgcgtcagc |
| | | | ECVSEDEEMSRVIPVI | gaagacgaagaaatgtctcgggtc |
| | | | ERITKELGVPISVDTY | attccggtcattgaacgcatcacaa |
| | | | KASVADEAVKAGASI | aggaactcggcgtcccgatttcagt |
| | | | INDIWGAKHDPKMA | ggatacatataaagcatctgtggca |
| | | | SVAAEHNVPIVLMH | gacgaagcagtcaaagcgggcgc |
| | | | NRPERNYNDLLPDM | atctattatcaatgacatttggggag |
| | | | LSDLMESVKIAVEAG | cgaaacatgatccgaagatggcaa |
| | | | VDEKNIILDPGIGFAK | gcgtcgcagcggaacataacgttc |
| | | | TYHDNLAVMNKLEIF | caattgtcctgatgcacaatcggcc |
| | | | SGLGYPVLLATSRKR | agaacggaattataacgaccttctt |
| | | | FIGRVLDLPPEERAEG | ccggatatgctgagcgaccttatg |
| | | | TGATVCLGIQKGCDI | gaatcagtcaaaattgcggttgag |
| | | | VRVHDVKQIARMAK | gcgggcgtggatgagaaaaatatt |
| | | | MMDAMLNKGGVHH | attttagatccgggcatcggcttcg |
| | | | G | cgaagacataccatgataatcttgc |
| | | | (SEQ ID NO. 83) | agtgatgaataagttagagatcttc |
| | | | | agcggacttggctatcctgtcctgc |
| | | | | tggctacatctcgtaaaagatttatc |
| | | | | ggaagagttcttgatttaccgcctg |
| | | | | aagagagagcagagggcacagg |
| | | | | agcgacagtctgcttgggcattca |
| | | | | gaaaggatgcgacatagtgcgtgt |
| | | | | tcatgatgtcaagcaaattgccaga |
| | | | | atggcgaaaatgatggacgcgatg |
| | | | | ctgaataagggaggggtgcaccat |
| | | | | ggatga (SEQ ID NO.: |
| | | | | 84) |
| folA/dfrA | Bacillus | NP_390064 | >NP_390064.1 | Atgatttcatttattttcgcaatgga |
| (dihydrofolate | subtilis | | dihydrofolate reductase | cgcgaatagactgataggcaaaga |
| reductase) | | | [Bacillus subtilis subsp. | caatgatctgccgtggcatttaccg |
| | | | subtilis str. 168] | aatgacctggcttattttaaaaaaat |
| | | | MISFIFAMDANRLIG | tacaagcggccatagcatcattatg |
| | | | KDNDLPWHLPNDLA | ggacgtaaaacatttgagtcaattg |
| | | | YFKKITSGHSIIMGRK | gcagacctcttccgaacagaaaaa |
| | | | TFESIGRPLPNRKNIV | acattgttgtcacatctgcgccgga |
| | | | VTSAPDSEFQGCTVV | ttcagaatttcagggctgcacagtc |
| | | | SSLKDVLDICSGPEEC | gtctcaagccttaaagacgttcttg |
| | | | FVIGGAQLYTDLFPY | atatttgcagcggaccggaagagt |
| | | | ADRLYMTKIHHEFEG | gttttgtcattggcggagcgcaatt |
| | | | DRHFPEFDESNWKLV | atacacagatctttttccgtacgcg |
| | | | SSEQGTKDEKNPYDY | gatagactgtatatgacaaaaatcc |
| | | | EFLMYEKKNSSKAG | accatgaatttgaaggcgacagac |
| | | | GF | actttcctgaatttgacgagagcaa |
| | | | (SEQ ID NO.: 85) | ctggaaactcgtgtctagcgaaca |
| | | | | gggcacgaaggatgagaaaaatc |
| | | | | cgtatgactatgaatttcttatgtatg |
| | | | | aaaagaaaaacagcagcaaagcg |
| | | | | ggaggcttttga |
| | | | | (SEQ ID NO.: 86) |

Engineered Strain and Preparation Method Thereof

The “engineered bacteria”, “engineered strain” and “genetically engineered strain” of the present invention can be used interchangeably, and both refer to reducing the expression level of the endogenous folC gene, and introducing the exogenous folC gene. In a preferred embodiment, folate synthesis regulatory genes (e.g., folE/mtrA, folB, folK, folP/sul, folA/dfrA) can also be introduced or upregulated.

Wherein, the engineered strain of the present invention has a significantly improved production capacity of a folate, a precursor thereof, or an intermediate thereof, compared with the starting strain, wherein the structure of the folate, the precursor, or the intermediate thereof is as shown in Formula I.

The starting strain that can be used to transform to the engineered strain of the present invention is a strain belonging to the genus Bacillus, particularly Bacillus subtilis. The synthesis ability of the folate, the precursor or the intermediate in the wild type starting strain is poor (Zhu et al., 2005), or it does not have the synthesis ability of the industrially required amount of folic acid, the precursor or the intermediate thereof. After genetic modification, in the engineered strain of the present invention, only one Glu residue is added to the produced folate, the precursor or the intermediate thereof, thereby enhancing the phenotype of folate excretion from the cell to the fermentation medium, and the production capability of folate, the precursor or the intermediate thereof is significantly increased, or this ability is greatly increased compared to the starting strain. Preferably, the “significantly increased” means that compared to its starting strain, the production capacity of the folate, the salt, the precursor or the intermediate thereof in the engineered strain is enhanced or increased by at least 100%, preferably, at least 200-50000%.

In addition, the starting strains that can be transformed to the engineered strains of the invention may also include the strains in the Table 3 below.

The engineered strain of the present invention can be obtained by the following methods:

(a1) knocking out an endogenous folC gene in a host cell;

(b1) cultivating the host cell; and

the method includes the steps of:

(a2) providing an expression vector carrying an exogenous folC gene;

(b2) transferring the expression vector into a host cell;

(c2) cultivating the host cell;

wherein the host cell is the starting strain.

Here we could have a section that any folate compound produced by the Bacillus subtilis strain, can then be converted to different derivatives, particularly folate using chemical steps and described in examples below.

Pharmaceutical Composition and Mode of Administration

The folate, the precursor or the intermediate thereof in the fermentation product of the strain of the present invention can be used for the preparation of a medication. The compounds of the invention may be administered to a mammal, such as a human, and may be administered orally, rectally, parenterally (intravenously, intramuscularly or subcutaneously), topically, and the like. The compounds can be administered alone or in combination with other pharmaceutically acceptable compounds. It is to be noted that the compounds of the present invention may be administered in combination.

Solid dosage forms for oral administration include capsules, tablets, pills, powders, and granules. In these solid dosage forms, the active compound is mixed with at least one conventional inert excipient (or carrier), such as sodium citrate or dicalcium phosphate, or mixed with the following components: (a) a filler or compatibilizer, for example, a starch, lactose, sucrose, glucose, mannitol and silicic acid; (b) binders such as hydroxymethylcellulose, alginates, gelatin, polyvinylpyrrolidone, sucrose and gum arabic; (c) humectants, for example, glycerin; (d) a disintegrant such as an agar, calcium carbonate, potato starch or tapioca starch, alginic acid, certain complex silicates, and sodium carbonate; (e) a slow solvent such as paraffin; (f) absorbing accelerators, for example, quaternary amine compounds; (g) wetting agents, such as cetyl alcohol and glyceryl monostearate; (h) adsorbents, for example, kaolin; and (i) lubricants, for example, talc, calcium stearate, magnesium stearate, solid polyethylene glycol, sodium lauryl sulfate, or a mixture thereof. In capsules, tablets and pills, the dosage form may also contain a buffer.

Solid dosage forms such as tablets, sugar pills, capsules, pills, and granules can be prepared with coatings and shells such as enteric coatings and other materials known in the art. They may contain opacifying agents and the release of the active compound or compound in such compositions may be released in a portion of the digestive tract in a delayed manner. Examples of embedding components that can be employed are polymeric and waxy materials. If necessary, the active compound may also be in microencapsulated form with one or more of the above-mentioned excipients.

Liquid dosage forms for oral administration include pharmaceutically acceptable emulsions, solutions, suspensions, syrups or elixirs. In addition to the active compound, the liquid dosage form may contain inert diluents conventionally employed in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil or a mixture of these substances.

In addition to these inert diluents, the compositions may contain adjuvants such as wetting agents, emulsifying and suspending agents, sweetening agents and perfumes.

In addition to the active compound, the suspension may contain suspending agents, for example, ethoxylated isostearyl alcohol, polyoxyethylene sorbitol and isosorbide dinitrate, microcrystalline cellulose, aluminum methoxide and agar or mixtures of these and the like.

Compositions for parenteral injection may comprise a physiologically acceptable sterile aqueous or nonaqueous solution, dispersion, suspension or emulsion, and a sterile powder for reconstitution into a sterile injectable solution or dispersion. Suitable aqueous and nonaqueous vehicles, diluents, solvents or excipients include water, ethanol, polyols and suitable mixtures thereof.

Dosage forms for the compounds of the present invention for topical administration include ointments, powders, patches, propellants and inhalants. The active ingredient is admixed under sterile conditions with a physiologically acceptable carrier and any preservatives, buffers, or, if necessary, propellants.

When a pharmaceutical composition is used, a safe and effective amount of a compound of the present invention is administered to a mammal (e.g., a human) in need of treatment wherein the dosage is a pharmaceutically effective dosage, for an individual of 60 kg body weight, the daily dose to be administered is usually from 1 to 1000 mg, preferably from 20 to 500 mg. Of course, the specific dose should also consider the route of administration, the health of the individual and other factors, which are within the skill of the skilled physician.

The main advantages of the invention include:

(1) A strain genetically engineered by the method of the present invention adds only one Glu residue on the produced the folate, the salt, the precursor or the intermediate thereof, thereby enhancing the phenotype of folic acid excretion from the cell to the fermentation medium, and can significantly increase the production capacity of folate, the precursor or the intermediate thereof; in addition, the strain is characterized by overexpression of folate biosynthetic genes, which further increase production capacity;

(2) The engineered strains are genetically stable and not susceptible to mutation;

(3) The engineered strains show comparable growth in standard fermentation media to other industrial B. subtilis strains.

The present invention is further described below with reference to specific embodiments. It should be understood that these examples are only for illustrating the present invention and not intended to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions described in the Journal of Microbiology: An Experimental Handbook (edited by James Cappuccino and Natalie Sherman, Pearson Education Press) or the manufacturer's proposed conditions. Unless otherwise specified, percentages and parts are percentages by weight and parts by weight.

Unless otherwise specified, the materials used in the examples are all commercially available products.

### Example 1: Identification of Folate Biosynthetic Genes in the Genome of Bacillus subtilis

Genes and enzymes involved in the folate biosynthetic pathway are known in the literature and are described in detail in the KEGG database (www.genome.jp/kegg/pathway.html). Nucleotide and protein sequences of key folate biosynthetic genes of B. subtilis were obtained by investigating the genome and protein databases of B. subtilis using the BLAST algorithm. Sequences of folate biosynthetic genes and enzymes were introduced as “query” and the corresponding B. subtilis sequences were identified as “hits.” Sequences of folate biosynthetic genes are presented in Table 2 below.

TABLE 2
Genes and enzymes involved in folate biosynthesis in Bacillus subtilis

| | | NCBI | Nucleotide | Protein |
|---|---|---|---|---|
| Gene | | accession | sequence | sequence |
| name: | Enzymatic activity | number | ID | ID |
| folE | GTP cyclohydrolase | NP_390159 | SEQ ID NO: 1 | SEQ ID NO: 7 |
| | (Bacillus subtilis) | | | |
| folB | 7,8-dihydroneopterin aldolase | NP_387959 | SEQ ID NO: 2 | SEQ ID NO: 8 |
| | (Bacillus subtilis) | | | |
| folK | 2-amino-4-hydroxy- | NP_387960 | SEQ ID NO: 3 | SEQ ID NO: 9 |
| | 6-hydroxymethyldihyd | | | |
| | ropteridine pyrophosphokinase | | | |
| | (Bacillus subtilis) | | | |
| sul | dihydropteroate synthase | NP_387958 | SEQ ID NO: 4 | SEQ ID NO: 10 |
| | (Bacillus subtilis) | | | |
| folC | bifunctional | NP_390686 | SEQ ID NO: 5 | SEQ ID NO: 11 |
| | folylpolyglutamate | | | |
| | synthetase/ | | | |
| | dihydrofolate synthetase | | | |
| | (Bacillus subtilis) | | | |
| dfrA | dihydrofolate reductase | NP_390064 | SEQ ID NO: 6 | SEQ ID NO: 12 |
| | (Bacillus subtilis) | | | |

### Example 2: Synthesis of Synthetic Genes for Folic Acid Biosynthesis, Optimized for Bacillus subtilis

The amino acid sequences (SEQ ID NOs: 7, 8, 9, 10 and 12) were used for gene codon optimization (Codon Optimization Tool from IDT Integrated DNA Technologies) in order to improve protein expression in B. subtilis. The synthesized DNA fragments (SEQ ID NOs: 13, 14, 15, 16 and 17, respectively) were designed with addition of RBS sequences, regulatory promoter sequence (such as p15 SEQ ID NO:38) for gene overexpression and short adapter sequences at both ends needed for further assembly of folic acid operon expression cassette.

### Example 3: Assembly of Folic Acid Operons

### Folic Acid Operon Assembled from Bacillus subtilis Genes

Key folate biosynthetic genes from Bacillus subtilis genes synthesized as DNA fragments (SEQ ID NOs: 13, 14, 15, 16 and 17) were used for assembly of folic acid operon (FOL-OP-BS2). For integration of folic acid operon into B. subtilis genome two additional DNA fragments with lacA homologies and erythromycin selectable marker (SEQ ID NO: 18 and 19) were designed and synthesized for stabile genome integration.

In the first step of the folic acid operon assembly PCR amplification of separate DNA fragments was performed with specific set of primers (primer pair SEQ ID NO:26 and SEQ ID NO:27 for fragment SEQ ID NO:13; primer pair SEQ ID NO:32 and SEQ ID NO:28 for fragment SEQ ID NO:17; primer pair SEQ ID NO:33 and SEQ ID NO:29 for fragment SEQ ID NO:15; primer pair SEQ ID NO:34 and SEQ ID NO:30 for fragment SEQ ID NO:16; primer pair SEQ ID NO:35 and SEQ ID NO:31 for fragment SEQ ID NO: 14).

Fragments were amplified using Eppendorf cycler and Phusion polymerase (Thermo Fisher) with buffer provided by the manufacturer with addition of 200 μM dNTPs, 5% DMSO, 0.5 μM of each primer and approximately 20 ng of template in a final volume of 50 μl for 32 cycles.

Used program: 98° C. 2 min
32 cycles of (98° C. 30 s, 65° C. 15 s, 72° C. 30 s)
72° C. 5 min
10° C. hold

PCR of each fragment was run on 0.8% agarose gel and cleaned from gel by protocol provided in Wizard PCR cleaning kit (Promega). The fragments were assembled into artificial folate operon by repetitive steps of restriction and ligation. A combination of NdeI and AseI restriction sites were used in order to assure compatible restriction ends for successful ligation. After each step of ligation, the combined fragments were used as a new template for next PCR amplification. Restriction was done in 50 μl volume with addition of 5 μl FD green buffer, 3 μl of selected enzyme and approximately 1500 ng of PCR fragment. Fragments were cleaned after restriction with Wizard SV Gel and PCR Clean-up system and first two were used in ligation. We used 2.5 U T4 DNA ligase (Thermo Fisher) with buffer provided by manufacturer and addition of 5% PEG 4000 and both fragments in 1:1 molar ratio to final volume 15 μl. In the next step 1 μl of inactivated ligation was used as a template in new 50 μL PCR with primers SEQ ID NO:26 and SEQ ID NO:28 and same program (with longer elongation time) and mix as used above. PCR was run on 0.8% agarose gel, fragment was excised from gel and cleaned. Cleaned new fragment (assembly of SEQ ID NO:13 and SEQ ID NO:17) was cut with Asel restriction enzyme and after additional cleaning used in ligation with third fragment (SEQ ID NO:15), already cut with Ndel and cleaned after. Following new PCR on ligation as a template, we also added fragment four and five by same protocol to make fragment of up to five folate biosynthetic genes.

Constructed folic acid operon assembled from Bacillus subtilis genes (shown in FIG. 4), was used for transformation (see Example 5) in order to generate strain FL722, after cultivation measurements of total folate was performed (see Example 13).

Folic Acid Operon from Lactococcus lactis subsp. lactis Genes

Heterologous genes (folA, clpX, ysxL, folB, folE, folP, ylgG and folC) from Lactococcus lactis subsp. lactis operon FOL-OP-LL (SEQ ID NO:49) were amplified by PCR and isolated genomic DNA was used as a template. Primers for PCR amplification were designed for two separate PCR reactions, where in the 1^{st} PCR reaction primers (SEQ ID NO:45 and SEQ ID NO:46) were used for specific amplification of genes from genomic DNA and in the 2^{nd} PCR reaction primers (SEQ ID NO:47 and SEQ ID NO:48) were used to additionally restriction sites (NheI and NotI) were introduced at both ends of the operon. The PCR product was subcloned into a low copy vector pFOL1 and the strong constitutive promoter P₁₅ (SEQ ID NO:38) was added at the start of the FOL-OP-LL operon. For construction of integration cassette for FOL-OP-LL operon, chloramphenicol resistance cassette and downstream homology for amyE locus was introduced. In the final step, the integration cassette was realised from cloning vector by using SbfI restriction enzyme and used for self-ligation to achieve multi copy genome integration. Constructed folic acid operon assembled from Lactococcus lactis subsp. lactis genes (shown in FIG. 2), was used for transformation in order to generate strain FL84, after cultivation measurements of total folate was performed (see Example 13).

Folic Acid Operon from Ashbya gossypii (Eremothecium gossypii) Genes

The expression cassette (FOL-OP-AG) from Ashbya gossypii (Eremothecium gossypii), a known B2 vitamin-producing filamentous fungus, was constructed using two synthetic folate biosynthesis genes, fol1-AG (SEQ ID NO:50) and fol2-AG (SEQ ID NO:51). The genes were codon-optimized for B. subtilis optimal expression and synthesized as two separate DNA fragments FOL1-AG (SEQ ID NO:52) and FOL2-AG (SEQ ID NO:53) where additional regulatory promoter sequence (promoter P₁₅) was introduced. The FOL1-AG fragment was first subcloned into a low copy vector pFOL1 using SpeI/BamHI restriction sites downstream of the chloramphenicol resistance cassette and strong constitutive promoter P₁₅. In the second step the FOL2-AG fragment was subcloned into a low copy vector pFOL2 upstream of the homology for amyE locus using EcoRV restriction site. In the next step DNA fragment containing P₁₅-fol2-AG and amyE homology was PCR amplified using primers (SEQ ID NO:54 and SEQ ID NO:55) and cloned into plasmid pFOL1 downstream of the chloramphenicol resistance cassette and P₁₅-fol1-AG using BamHI restriction site. In the final step, the assembled integration cassette FOL-OP-AG was PCR amplified using primers (SEQ ID NO:56 and SEQ ID NO:57) and PCR product was used for transformation of the cell. Constructed folic acid operon assembled from Ashbya gossypii genes (shown in FIG. 3), was used for transformation in order to generate strain FL260, after cultivation measurements of total folate was performed (see Example 13).

### Example 4: Assembly of Genetic Construct for folC Replacement

In order to replace the native folylpolyglutamate synthase (folC), which is capable of attaching multiple glutamate residues to folates, with the variant, capable of attaching only the first glutamate residue in folate biosynthesis we set out to generate the corresponding genetic constructs. The folC disruption cassettes were assembled by using folC homology ends amplified by PCR from gDNA B. subtilis VBB38 by using the corresponding primer pairs SEQ ID NO:43 and SEQ ID NO:44. PCR mix was made with Phusion polymerase (Thermo Fisher) and buffer provided by manufacturer with addition of 5% DMSO, 200 μM dNTPs and 0.5 μM of each primer to final volume of 50 μL for 32 cycles (annealing temperature 65° C., elongation time 2 min). The amplified PCR fragment was excised from 0.8% agarose gel, cleaned with Wizard Gel and PCR Clean-up system kit and phosphorylated with T4 polynucleotide kinase (Thermo Fisher) in buffer A, provided by manufacturer, with addition of 1 mM ATP.

Prepared fragment was ligated in low copy plasmid pET-29c (Novagen), which was previously cut with FspAl and Xhol, blunt-ended with DNA polymerase 1, Large (Klenow) fragment (Thermo Fisher) and dephosphorylated with FastAP Thermosensitive Alkaline Phosphatase (Thermo Fisher).

Tetracycline resistance cassette (SEQ ID NO:21) was used to disrupt folC gene sequence. Tetracycline resistance cassette was inserted into folC sequence by cutting plasmid with Bsp119l restriction enzyme, blunt-ended with DNA polymerase 1, Large (Klenow) fragment (Thermo Fisher), dephosphorylated, using FastAP and ligated using T4 DNA ligase (Thermo Fisher).

Further, heterologous folC2 protein sequences from Lactobacillus reuteri (folC2-LR) (SEQ ID NO:22) and from Ashbya gossypii (folC2-AG) (SEQ ID NO:23) were used for design codon optimized DNA sequence for folC2-LR (Lactobacillus reuteri) (SEQ ID NO:24) and for folC2-AG (Ashbya gossypii) (SEQ ID NO:25) heterologous gene expression. DNA fragments were synthesized (IDT Integrated DNA Technologies) and used for construction of two integration cassettes (shown in FIG. 5). First, we generated a blunt-ended fragment containing the Pveg promotor (SEQ ID NO:37) using DNA polymerase 1, Large (Klenow) fragment (Thermo Fisher) and ligated it in the plasmid with folC homology, previously cut with Xbal and blunt-ended with DNA polymerase 1, Large (Klenow) fragment (Thermo Fisher).

Next, newly constructed plasmid was cut with Bcul and FspAl restriction enzymes and dephosphorylated, using FastAP. After that, plasmid was ligated with ordered optimized sequences folC2-AG in folC2-LR, previously cut with Bcul and FspAl restriction enzymes. In this plasmid tetracycline resistance, previously cut with EcoRl restriction enzyme and blunt-ended, was ligated, after restriction of plasmid with FspAl and dephosphorylated. Constructed plasmids were used as a template for PCR primers SEQ ID NO:43 and SEQ ID NO:44 in order to generate folC disruption/replacement cassette for transformation.

### Example 5 Assembly of Folic Acid Operon Constructs for Transformation

After assembly of folic acid operon (see Example 3) DNA fragments with folate biosynthetic genes were further cut with Xbal restriction enzyme and ligated with synthetized DNA fragment for erythromycin resistance cassette (SEQ ID NO:58) with primers SEQ ID NO:40 and SEQ ID NO:41 (62° C., 40 s) and cut with XbaI to ensure compatible DNA ends for ligation. After ligation whole fragment was PCR amplified with primers (SEQ ID NO:36 and SEQ ID NO:39).

In the final step of assembly fragment (SEQ ID NO:18) with lacA homology and regulatory promoter region was added. Fragments were cut with Spel restriction enzyme and used in ligation. Ligation mixture was used as PCR template with primers (SEQ ID NO:42 and SEQ ID NO:39), with which we finish assembly of artificial folate operon (shown in FIG. 4) as an expression cassette (SEQ ID NO:20) for genome transformation into B. subtilis strains.

### Example 6: Selection of Possible Bacillus subtilis Host Strains for Engineering of Folate Production

Different Bacillus strains can be used as starting strains for engineering of folate production (Table 3). Bacillus strains can be isolated from nature or obtained from culture collections. Among others, starting strains for folate production can be selected among Bacillus subtilis strains that have already been subjected to classical methods of mutagenesis and selection in order to overproduce metabolites related to the purine biosynthetic pathway. For example, strains overproducing riboflavin, inosine and guanosine may be selected. Strains subjected to random mutagenesis and toxic metabolic inhibitors from purine and riboflavin pathway are preferred and are included in Table 3.

TABLE 3
Potential non-GMO starting strains of B. subtilis
that could be used for development of folate production.

| | | Alternative | | | |
|---|---|---|---|---|---|
| Species | Name | name | Product | Availability | Remarks |
| B. subtilis | 168 | ATCC6051 | none | yes | Type strain |
| B. subtilis | W23 | ATCC 23059/ | none | yes | Type strain |
| subsp. | | NRRLB-14472 | | | |
| spizizenii | | | | | |
| B. subtilis | RB50 | NRRL B18502 | riboflavin | yes | Developed by |
| | | | | | Roche/DSM |
| B. subtilis | RB58 | ATCC55053 | riboflavin | yes | Containing |
| | | | | | additional copy |
| | | | | | of rib operon |
| B. subtilis | VNII | VKPM B2116, | riboflavin | yes | Developed by |
| | Genetika 304 | VBB38 | | | VNII Genetika |
| B. subtilis | FERM-P | | riboflavin | no | Ajinomoto |
| | 1657 | | | | |
| B. subtilis | FERM-P | | riboflavin | no | Ajinomoto |
| | 2292 | | | | |
| B. subtilis | AJ12644 | FERM BP-3855 | riboflavin | no | Ajinomoto |
| B. subtilis | AJ12643 | FERM BP-3856 | riboflavin | no | Ajinomoto |
| B. subtilis | ATCC13952 | | inosine | yes | |
| B. subtilis | ATCC19221 | IFO 14123 | guanosine | yes | |
| B. subtilis | ATCC13956 | IFO 14124 | inosine | yes | |

VKPM B2116 strain is a hybrid strain of B. subtilis 168 strain (most common B. subtilis host strain with approx. 4 Mbp genome) with a 6.4 kbp island of DNA from the strain B. subtilis W23. Such architecture is common for most B. subtilis industrial strains and was obtained by transforming the 168 strain (tryptophan auxotroph trpC−) with W23 (prototrophic TrpC+) DNA. It has a 6.4 kbp W23 island in the genome, which is the same as in the commonly used strain B. subtilis SMY, which is one of the B. subtilis legacy strains with genome publicly available (Ziegler et al., The origins of 168, W23 and other Bacillus subtilis legacy strains, Journal of Bacteriology, 2008, 21, 6983-6995). VKPM B2116 strain is a direct descendant of the SMY strain, obtained by classical mutagenesis and selection. Another name for this strain is B. subtilis VNII Genetika 304. The description of construction of the strain in described in Soviet Union patent SU908092, filed in 1980. The mutations were obtained by subsequent mutagenesis and selection on metabolic inhibitors. The strain VKPM B2116 is resistant to roseoflavin, a toxic analogue of vitamin B2, due to a mutation in the ribC gene, encoding a flavin kinase. This strain is also resistant to 8-azaguanine, toxic analogue of purine bases.

### Example 7: Replacement of folC and Generation of the Optimum Host Strain for Folic Acid Production

After construction of heterologous folC2 (folC2-AG or folC2-LR) gene expression cassette (see example 4 and FIG. 5) we have performed transformation of B. subtilis VBB38 and B. subtilis VBB38Δrib. Expression cassette with homologies for native folC gene disruption, was amplified by PCR with primers SEQ ID NO:43 and SEQ ID NO:44. After transformation colonies resistant to tetracycline were selected and native folC gene replacement, by a heterologous folC2 gene (A. gossypii or L. reuteri), was genetically confirmed with cPCR and sequencing of obtained PCR product. New strains were used to test the production yields of the total folates (see FIG. 18), and to compare the distribution of the total folates between the supernatant and the cell biomass.

### Example 8: Transformation of Bacillus subtilis

i) Bacillus subtilis natural competence transformation

10 mL of SpC medium is inoculated from fresh plate of B. subtilis and cultured overnight. 1.3 mL of overnight culture is diluted into 10 mL of fresh SpC medium (9× dilution). OD450 is measured and is expected to be around 0.5. Cultures are grown for 3 h 10 min at 37° C. 220 RPM. OD450 is measured again and is expected to be between 1.2-1.6. Cultures are diluted 1:1 with SpII (starvation medium). 3.5 ml of culture is mixed with 3.5 ml of starvation medium and tryptophan in concentration 50 ug/ml is added. Cultures are grown for additional 2 h at 37° C., 220 RPM. After incubation cultures are maximally competent for 1 h. 500 μl of competent cells is mixed with DNA (5-20 μl, depending on concentration) in 2 mL Eppendorf tube and incubated for 30 min at 37° C. with shaking. 300 μl of fresh LB is added for the recovery of competent cells and incubated for additional 30 min at 37° C. Eppendorf tubes are centrifuged at 3000 RPM, 5 min. Pellet is resuspended and plated on LB plates with appropriate antibiotic.

Medium:

10× T-base

150 mM ammonium sulfate

800 mM K₂HPO₄

440 mM KH₂PO₄

35 mM sodium citrate

SpC (minimal culture media)

100 mL 1× T-base

1 mL 50% glucose

1.5 mL 1.2% MgSO₄

2 ml 10% yeast extract

2.5 ml 1% casamino acids

SpII (starvation media)

100 ml 1× T-base

1 ml 50% glucose

7 ml 1.2% MgSO₄

1 ml 10% yeast extract

1 ml 1% casamino acids

0.5 ml 100 mM CaCl₂

### Example 10: Determination of Folate Operon Copy Number Using qPCR

We used real time quantitative PCR (qPCR) technique for determination of the number of copies of the integrated B. subtilis artificial folate operon genes. The copy numbers of the genes folP, folK, folE, dfrA and KnR (the gene for kanamycin resistance) in the artificial folate operon in the folate-producing B. subtilis transformants was estimated by (qPCR) with SYBR Green I detection. The copy number of the gene for kanamycin resistance (KnR) and the copy number of the folate biosynthesis genes folP, folK, folE, dfrA on artificial B. subtilis folate operon were quantified by qPCR. Genomic DNA of the B. subtilis strains was isolated with SW Wizard Genomic DNA Purification Kit (Promega). The concentration and purity of gDNA were evaluated spectrophotometrically at OD260 and OD280. The amount of gDNA used in all experiments was equal to the amount of gDNA of the reference strain. A B. subtilis with a single copy of artificial folate operon containing the genes folP, folK, folE, dfrA and KnR was used as a reference strain for relative quantification of the gene copy numbers. A housekeeping gene DxS, a single-copy gene in the B. subtilis genome, was used as the endogenous control gene. Quantification of gene copy number for the folate biosynthesis genes was performed using specific set of primers (primer pair SEQ ID NO:59 and SEQ ID NO:60 for folP gene, primer pair SEQ ID NO:61 and SEQ ID NO:62 for folK gene, primer pair SEQ ID NO:63 and SEQ ID NO:64 for folE gene, primer pair SEQ ID NO:65 and SEQ ID NO:66 for dfrA gene) for quantification of kanamycin resistance marker attached to folate operon (primer pair SEQ ID NO:67 and SEQ ID NO:68) and for reference DxS gene primer pair SEQ ID NO:71 and SEQ ID NO:72 were used. The qPCR analysis was run on StepOne™ Real-Time PCR System and quantification was performed by using the 2^{-ΔΔCT} method.

The gene copy numbers of the genes in the artificial BS-FOL-OP strains were quantified relatively to the strain with one copy of the genes. The KnR gene of the B. subtilis strain with one copy number was used as the reference strain for relative quantification of the gene copy numbers of genes in the artificial folate operon in B. subtilis transformed strains. The qPCR relative quantification of the genes folP, folK, folE, dfrA and KnR genes showed 6-fold increase in RQ values compared to B. subtilis strain with single copy genes. Folate overproducing strains FL179 and FL722 were confirmed to have multi-copy integration of folic acid synthetic operon.

### Example 11: Cultivation of Bacillus subtilis Strains

Serial dilutions from frozen cryovial are made and plated on to MB plates with appropriate antibiotic and incubated for approximately 48 h at 37° C. For further testing use at least 10-20 single colonies from MB plates for each strain. First re-patch 10-20 single colonies on fresh MB plates (with the same concentration of antibiotics) for testing.

For vegetative stage MC medium is used and inoculated with 1 plug per falcon tube (or 5 plugs per baffled Erlenmeyer flask or small portion of patch for microtiter plates). Appropriate antibiotics are added into medium. For microtiter plates 500 μl of medium is used in 96 deep well, for falcon tubes is used 5 ml of medium (in 50 ml falcon tube) and for Erlenmeyer flask 25 ml (in 250 ml flask). Cultures are incubated at 37° C. for 18-20 h at 220 RPM.

Inoculation into production medium (MD) is after 18-20 h in vegetative medium. 10% inoculum is used (50 μl for MW, 0.5 ml for falcon tube and 2.5 ml Erlenmeyer flask). Each strain is tested in two aliquots. For microtiter plates 500 μl of medium is used in 48 deep well, for falcon tubes is used 5 ml of medium and for baffled Erlenmeyer flask 25 ml. Wires are used in falcon tubes for better aeration, as are gauzes used instead of the stoppers on Erlenmeyer flasks. Cultures are incubated at 37° C. for 48 h at 220 RPM. After 24 and 48 hours titer of total folates was measured using the microbiological assay, according to the developed procedures

Best candidate strains are retested in the same manner and after several confirmations prepared for testing in bioreactors. 100 μl of frozen culture of selected strain for bioreactor testing is spread on to MB plates with appropriate antibiotic and incubated for approximately 48 h at 37° C. Complete biomass is collected with 2 ml of sterile 20% glycerol per plate. Collected biomass is distributed into 100 μl aliquots and frozen at −80° C. This is used as working cell bank for bioreactor testing.

Medium Composition:

1) MB (plates)

Trypton 10 g/l

Yeast extract 5 g/l

NaCl 5 g/l

Maltose 20 g/l

Agar 20 g/l

pH 7.2-7.4

Autoclaved 30 min, 121° C.

After autoclaving and cooling down appropriate antibiotics are added.

2) MC (Vegetative Medium)

Molasses 20 g/l

CSL 20 g/l

Yeast extract 5 g/l

MgSO₄*7H₂O 0.5 g/l

(NH₄)₂SO₄ 5 g/l

Ingredients are mixed together and pH set to 7.2-7.4. KH₂PO₄—K₂HPO₄ solution is then added in final concentration for KH₂PO₄ 1.5 g/l and K₂HPO₄ 3.5 g/l. Medium is distributed into falcon tubes (5 ml/50 ml-falcon tubes) or Erlenmeyer flasks (25 ml/250 ml-baffled Erlenmeyer flask) and autoclaved 30 min, 121° C. Sterile glucose is added after autoclaving in final concentration 7.5 g/l. Antibiotics are added prior to inoculation.

3) MD (production medium)

Yeast 20 g/l

Corn steep liquor (CSL) 5 g/l

MgSO₄*7H₂O 0.5 g/l

para-aminobenzoic acid (pABA) 0.5 g/L

Ingredients are mixed together and pH set to 7.2-7.4. KH₂PO₄—K₂HPO₄ solution is then added in final concentration for KH₂PO₄ 1.5 g/l and K₂HPO₄ 3.5 g/l The medium is autoclaved at 121° C. for 30 min. Sterile urea solution (20 ml of stock solution, final concentration is 6 g/L), sterile glucose solution (250 ml of stock solution, final concentration is 100 g/L glucose), sterile pABA solution (100 ml of stock solution, final concentration is 0.5 g/L) and 150 ml of sterile water are added after autoclaving to obtain 1 L of MD+pABA500 medium. Appropriate antibiotics were added prior to inoculation. Medium is then distributed into sterile Erlenmeyer flasks (25 ml/250 ml-baffled Erlenmeyer flask.

### Example 12: Microbiological Assay for Quantification of Total Folates in Fermentation Broths

A microbiological assay using Enterococcus hirae NRRL B-1295 was used for detection of the total folates produced in the strains of Bacillus subtilis. The microbiological assay was used for the evaluation of the intracellular (retained in the biomass) and extracellular (released into the culture medium) total folates produced by B. subtilis. For the microbiological assay, the indicator organism Enterococcus hirae NRRL B-1295 is used, which is auxotrophic for folates or folic acid. E. hirae is precultured in the rich growth medium, containing folates (Lactobacilli AOAC broth) at 37° C. for 18-24 h. It is then washed in the growth medium without folates (folic acid assay medium) to remove the residual folates. The washed E. hirae culture is inoculated into the assay medium without folic acid. The microbiological assay is set up in 96-well microtiter plates. Appropriately diluted media samples to be assayed and the standard solutions of folic acid are added to the growth medium containing the indicator strain, and the plate is incubated at 37° C. for 20 h. The growth response of the indicator organism is proportional to the amount of folic acid/folates present in the media samples/controls. The standard curve is constructed for each assay by adding a set of standard solutions of folic acid to the growth medium and the indicator strain. The growth is measured by measuring the optical density (OD) at 600 nm wavelength. The growth response of E. hirae to the test samples is compared quantitatively to that of the known standard solutions. A dilution series containing various concentrations of folic acid is prepared and assayed as described above. The standard curve is obtained by plotting the measured OD₆₀₀ at known concentrations of folic acid. The standard curve is used to calculate the amounts of total folates in the test samples. The indicator organism E. hirae NRRL B-1295 is used to detect the concentrations of total folates in the range from 0.05 to 0.7 ng/mL in the measured sample. The total extracellular and intracellular folates produced by B. subtilis strains can be estimated by adding appropriately diluted test samples to the indicator organism E. hirae in folic acid assay medium.

### Example 13: Analysis of Total Folate Yields of Different Starting Strains and Initial folC-Replaced and Folic Acid Operon Amplified Strains

The transformants in which folC gene was replaced by a heterologous folC2 gene from either A. gossypii (B. subtilis strain FL21) or L. reuteri (B. subtilis strain FL23) and transformants with amplified folic acid operon were tested for total folate amounts at the shaker scale (5 ml production medium MD). After the fermentation, the samples of the fermentation broth (200 μl) was carefully collected to obtain a homogeneous sample and diluted 10 times in the ice-cold extraction buffer (0.1 M phosphate buffer with 1% (w/v) ascorbic acid). The samples were centrifuged at 14,000 rpm and 4° C. for 10 min and filter-sterilized (0.22 μm pore size). For the microbiological assay samples were serially diluted in the extraction buffer and kept at 4° C. until the microbiological assay was set up. In the Table 4 results for selected strains measured by the microbiological assay are presented.

TABLE 4
Total folate production of different Bacillus
subtills strains in experiments at shaker scale (5 ml)

| | Total folate |
|---|---|
| | production |
| Description of strain | (mg/L) |
| B. subtilis w.t. 168 | 0.31 |
| VBB38 (B. subtilis VKPM B2116) | 1.24 |
| FL23 (B. subtilis VBB38 folC::tetR P-veg/folC2-LR) | 3.4 |
| FL1027 (B. subtilis VBB38 folC::tetR P-veg/folC2-LR; | 238.0 |
| lacA::ermAM P-15/FOL-OP-BS2) | |
| FL21 (B. subtilis VBB38 folC::tetR P-veg/folC2-AG) | 6.1 |
| FL260 (B. subtilis VBB38 folC::tetR P-veg/folC2-AG; | 45.7 |
| amyE::cat P-15/FOL-OP-AG) | |
| FL84 (B. subtilis VBB38 folC::tetR P-veg/folC2-AG; | 55.2 |
| amyE::cat P-15/FOL-OP-LL) | |
| FL179 (B. subtilis VBB38 folC::tetR P-veg/folC2-AG; | 573.0 |
| amyE::kanR P-veg/FOL-OP-BS1) | |
| FL722 (B. subtilis VBB38 folC::tetR P-veg/folC2-AG; | 587.4 |
| lacA::ermAM P-15/FOL-OP-BS2) | |

### Example 14: Determination of Concentrations Folate Forms and Related Compounds Using LC-MS and Identification of 10-Formyl-Dihydrofolic Acid and 10-Formyl Folic Acid as Two Main Products

In addition to the microbiological assay, our aim was to develop sensitive and versatile analytical method, with reasonably short analytical run time. The method had to be LCMS compatible with volatile mobile phase, and also had to enable UV detection and give good chromatographic separation of as many folate-related analytes as possible.

Instruments and Materials:

The method was developed on Thermo Accela 1250 HPLC instrument with PDA detector, coupled with MS/MS capable mass spectrometer Thermo TSQ Quantum Access MAX, equipped with hESI source. Method has been set-up on Thermo Acclaim RSLC PA2, 150×2.1 mm HPLC column with 2.2 μm particle size. PDA detector is set at 282 nm, with bandwidth 9 nm and 80 Hz scan rate, and also DAD scan from 200-800 nm. Column oven is set at 60° C. and tray cooling at 12° C. Injection solvent is 10% methanol in water, with wash and flush volume: 2000 μl. Injection volume is set at 10 μl and can also be set at 1 μl when higher concentrations of analytes are expected. Mobile phase A is 650 mM acetic acid in water, and mobile phase B is methanol. Mobile phase flow is 0.5 ml/min and total run time is 20 min. Method is using gradient program in Table 5 and MS spectrometer parameters described in Table 6.

TABLE 5
Gradient program for the chromatographic analysis

| Time/min | % A | % B |
|---|---|---|
| 0.00 | 100 | 0 |
| 2.00 | 100 | 0 |
| 16.00 | 82 | 18 |
| 16.01 | 100 | 0 |
| 20.00 | 100 | 0 |

TABLE 6
MS spectrometer tune parameters and other MS/MS relevant parameters

| Tune parameters: | | Other method parameters: | |
|---|---|---|---|
| Ionization | hESI + | Polarity: | Positive |
| Spray voltage | 4000 V | Scan width | 5.000 |
| | | (m/z): | |
| Vaporizer temperature | 350° C. | Scan time (s): | 0.100 |
| Sheath gas pressure | 55 | Q1 (FWHM): | 0.70 |
| Aux gas pressure | 5 | Micro scans: | 1 |
| Capillary temperature | 300° C. | Data type: | Centroid |
| Tube lens offset | SRM table | Chrom filter | none |
| | | P.W. | |
| Skimmer offset | 0 | MS Acquire | 15 |
| | | time (min): | |
| Collision pressure | 1.0 torr | Divert valve: | none |

LCMS detector is coupled after DAD detector, and analytes are observed in scan from 400-600 m/z mode, in SIM mode at their M.W.+1 and MS/MS mode (Table 6). Standards were prepared with weighting and dissolving in 0.1 M NaOH solution (Table 7 and Table 8) and immediately put to HPLC instrument.

TABLE 7
Available standards

| Analyte: | Purity: | Source: | Abbreviation: |
|---|---|---|---|
| Folic acid | 91.3% | Pharmacopoeia | FA |
| Dihydro folic acid | >80.0% | Sigma | DHF |
| Tetrahydro folate | >65.5% | Sigma | THF |
| 5-methyl | >81.0% | Carbosynth | 5M-THF, 5-methyl THF |
| tetrahydro folate | | | |
| 10-formyl folic acid | 91.4% | EDQM | 10F-FA, 10-formyl FA |
| 5-formyl | >90.0% | EDQM | 5F-THF, 5-formyl THF |
| tetrahydro folate | | | |

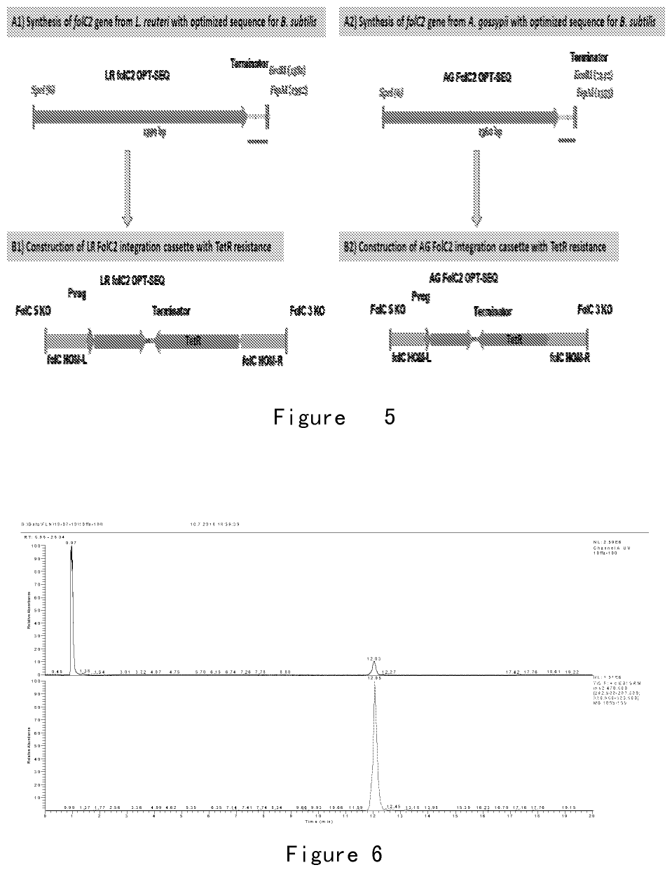

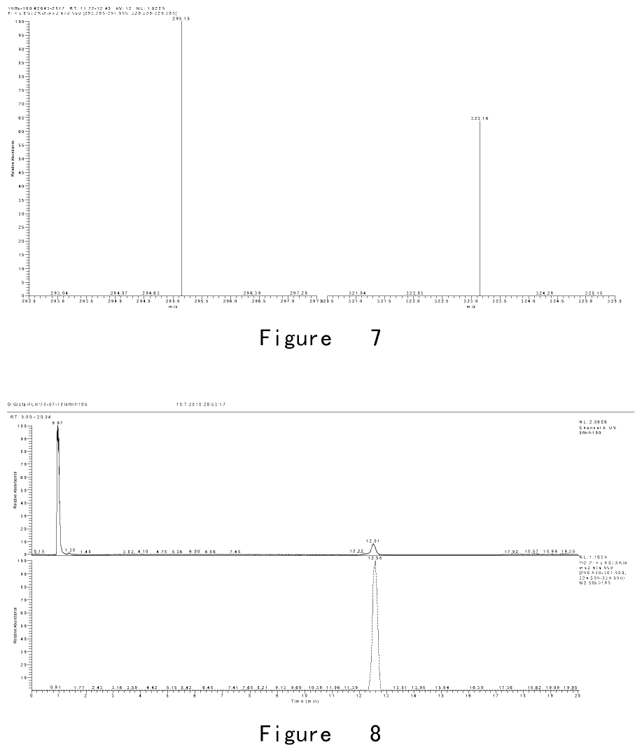

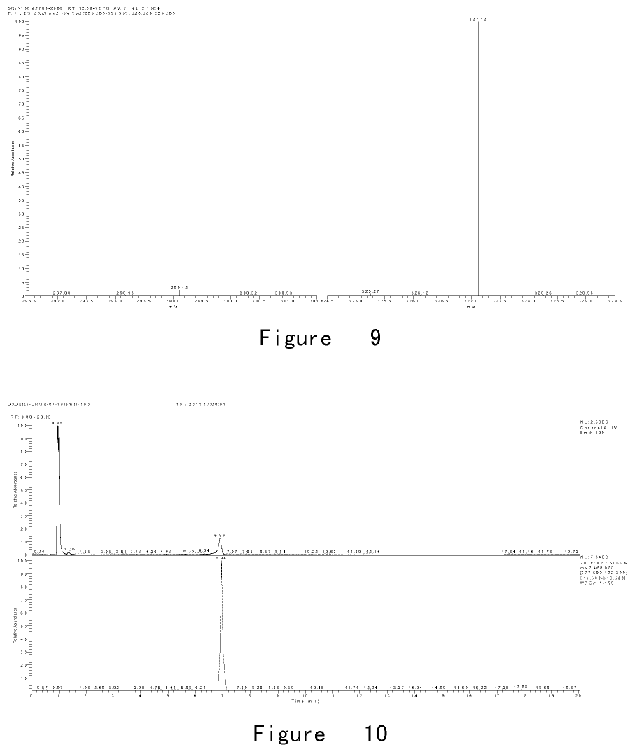

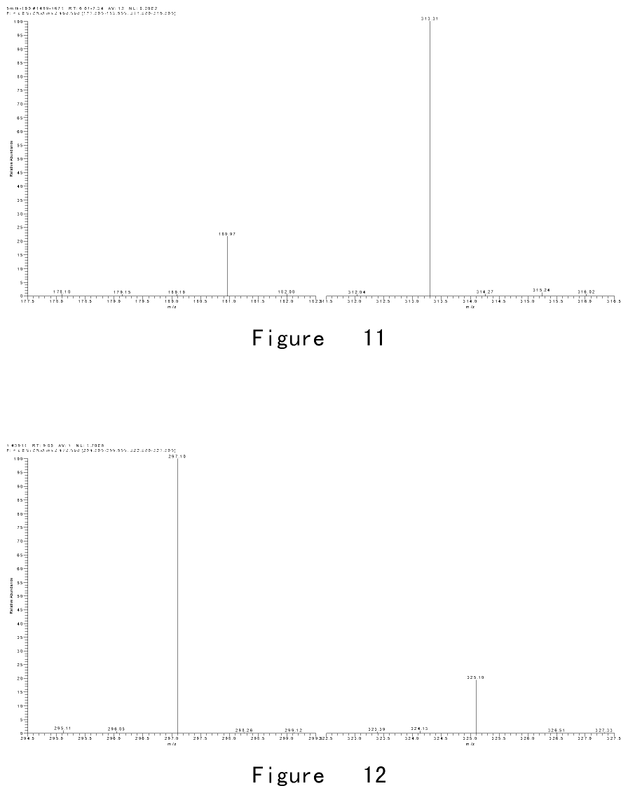

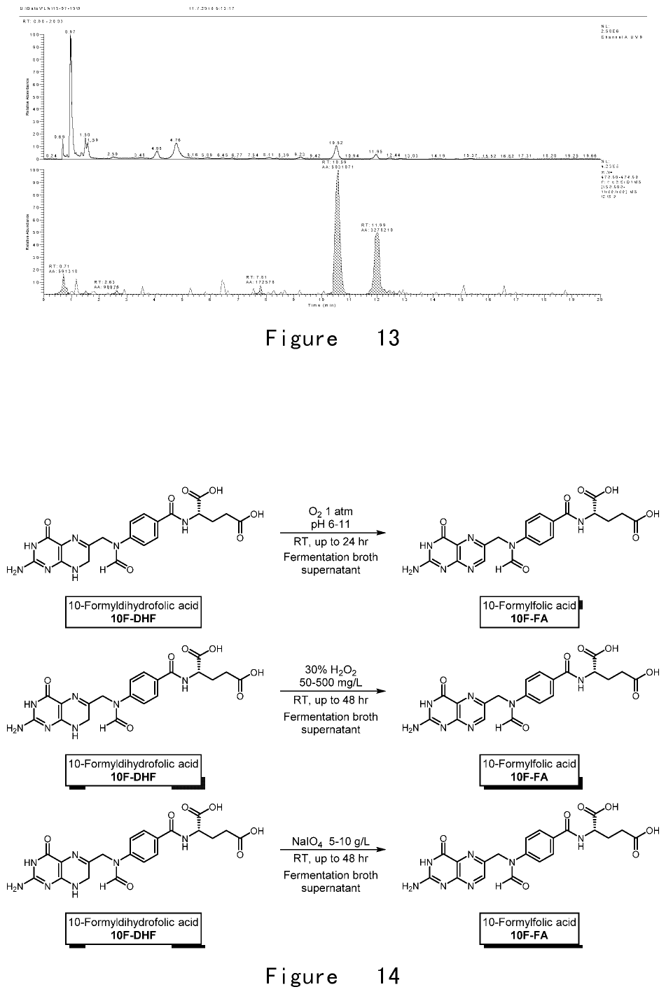

TABLE 8
Observed standards and their related MS/MS method settings

| | Parent | Product(s) | | Tube | |
|---|---|---|---|---|---|
| Analyte: | m/z: | m/z: | Collision E: | lens: | Chromatogram |
| Folic acid | 442 | 295 | 19 | 50 | |
| Dihydro f.a. | 444 | 178, 297 | 19 | 50 | |
| Tetrahydro f.a. | 446 | 299, 318, 361, 387 | 20 | 50 | |
| 5-methyl THF | 460 | 180, 314 | 20 | 50 | FIG. 10 |
| | | | | | and |
| | | | | | FIG. 11 |
| 10-formyl f.a. | 470 | 295, 323 | 20 | 50 | FIG. 6 |
| | | | | | and |
| | | | | | FIG. 7 |
| 10-formyl | 472 | 297, 325 | 20 | 50 | FIG. 12 |
| dihydro f.a. | | | | | |
| 5-formyl THF | 474 | 299, 327 | 20 | 50 | FIG. 8 |
| | | | | | and |
| | | | | | FIG. 9 |

Method has linear response for MS/MS detection up to 1000 mg/L of analyte, with correlations above 90% for all standards.

### Example 15: Different Ratio of Folic Acid and Derivatives Production Through Genetically Modified Bacillus subtilis

The transformants in which folC gene was replaced by a heterologous folC2 gene from either A. gossypii (B. subtilis strain FL21) or L. reuteri (B. subtilis strain FL23) and transformants with amplified folic acid operon were tested for total folate amounts at the shaker scale (5 ml production medium MD).

The strains were patched on MB plates with appropriate antibiotics and incubated at 37° C. for 2 days. For shake-flasks experiments, the grown strains were transferred to 5 ml of MC (seed) medium in Falcon 50 mL conical centrifuge tubes (1 plug/5 ml) and cultivated on a rotary shaker at 220 RPM and 37° C. for 16-18 h. A 10% inoculum of the seed culture was used to inoculate 5 mL of the production medium (MD+pABA500). The strains were cultivated on a rotary shaker at 220 RPM and 37° C. for 48 h in the dark. After the fermentation, the samples of the fermentation broth (200 μl) was carefully collected to obtain a homogeneous sample and diluted 10 times in the ice-cold extraction buffer (0.1 M phosphate buffer with 1% (w/v) ascorbic acid). The samples were centrifuged at 14,000 rpm and 4° C. for 10 min and filter-sterilized (0.22 μm pore size). For the quantification of different folate species HPLC method was used as described in Example 14. Results of different B. subtilis strain are shown in Table 9 and representative HPLC chromatogram of fermentation broth sample is shown in FIG. 13.

TABLE 9
Total folate production of different Bacillus subtills
strains in experiments at shaker scale (5 ml)

| | | 5M-THF | FA | 10F-DHF | 10F-FA |
|---|---|---|---|---|---|
| Strain | Strain description | (mg/L) | (mg/L) | (mg/L) | (mg/L) |
| wt 168 | B. subtillis wild type | 0.16 | 0.15 | 0.01 | 0.86 |
| VBB38 | B. subtilis VKPM B2116 | 0.35 | 0.01 | 0.02 | 0.09 |
| FL 23 | VBB38 folC::tetR P-veg/folC2-LR | 1.11 | 0.01 | 0.01 | 0.03 |
| FL 21 | VBB38 folC::tetR P-veg/folC2-AG | 18.20 | 0.01 | 0.03 | 2.64 |
| FL 84 | FL21 amyE::cat P-15/FOL-OP-LL | 25.46 | 0.02 | 0.34 | 18.81 |
| FL 260 | FL21 amyE::cat P-15/FOL-OP-AG | 21.67 | 0.04 | 1.52 | 44.50 |
| FL 179 | FL21 amyE::kanR P-veg/FOL-OP-BS1 | 97.05 | 0.03 | 16.22 | 373.22 |
| FL 722 | FL21 lacA::ermAM P-15/FOL-OP-BS2 | 20.21 | 0.30 | 13.21 | 351.00 |

Strain FL179 with heterologous folC-AG and overexpressed folate biosynthetic genes from B. subtilis showed 43297% increased 10-formyl folic acid production compared to the wild type strain Bacillus subtilis 168.

### Example 16: Oxidative Conversion of 10-Formyldihydrofolic Acid to 10-Formyl Folic Acid

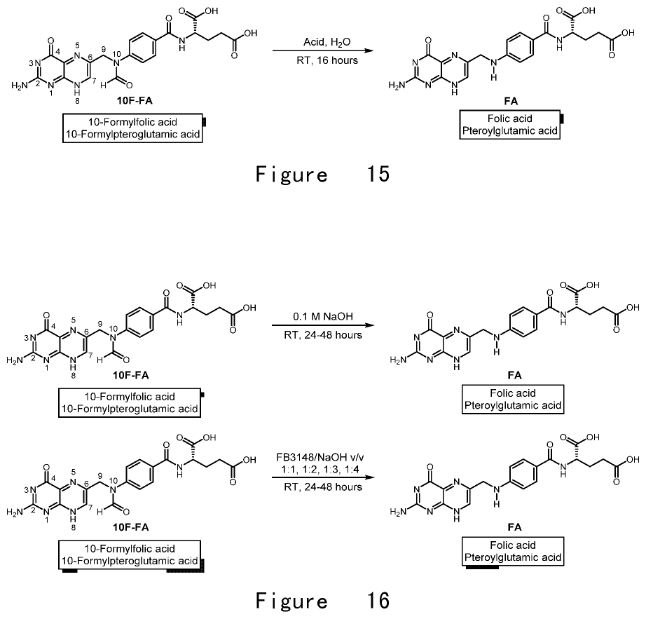

At the end of the fermentation, HPLC analysis of broth detected a relatively high amount (85 Area %) of 10-formyldihydrofolic acid (10F-DHF). Furthermore, we observed that 10-formyldihydrofolic acid can be oxidatively converted to 10-formylfolic acid (see FIG. 14). Accordingly, we started to develop a protocol, which will provide a quantitative conversion to 10-formylfolic acid. We anticipate the subsequent deformylation step will provide a folic acid in the highest possible yield. Literature search revealed a report describing the oxidation of tetrahydrofolic acid by air in aqueous solutions at specific pH values (Reed1980). Based on this report, at pH values 4, 7 and 10 the major products of oxidation are p-aminobenzoylglutamic acid (PABG) and 6-formylpterin. In addition, 7,8-dihydrofolate intermediate was only detected at pH=10. We carried out the series of oxidation experiments on the fermentation broth supernatant to facilitate a swift conversion of 10-formyldihydrofolic acid to 10-formylfolic acid. We examined several oxidation reagents such as O₂, H₂O₂ and NaIO₄ (see FIG. 14).

TABLE 10
Effect of pH on oxidation of 10-formyldihydrofolic acid to
10-formylfolic acid in the fermentation broth supernatant with oxygen

| | | | | | | | | SUM |
|---|---|---|---|---|---|---|---|---|
| | | | | | 10F-DHF | 10F-FA | FA | FOL |
| exp | pH | oxidant | time | temp | mg/L | mg/L | mg/L | mg/L |
| 1 | 7 | — | 0 hr | 25° C. | 782.9 | 180.2 | 12.4 | 975.5 |
| 2 | 6 | O₂ 1atm | 48 hr | 25° C. | 140.0 | 368.2 | 7.3 | 515.5 |
| 3 | 7 | O₂ 1atm | 48 hr | 25° C. | 253.5 | 366.1 | 10.3 | 611.9 |
| 4 | 8 | O₂ 1atm | 48 hr | 25° C. | 268.1 | 376.5 | 12.0 | 656.5 |
| 5 | 9 | O₂ 1atm | 48 hr | 25° C. | 199.4 | 293.6 | 0 | 493 |
| 6 | 10 | O₂ 1atm | 48 hr | 25° C. | 80.7 | 288.4 | 0 | 369.1 |
| 7 | 11 | O₂ 1atm | 48 hr | 25° C. | 0 | 351.3 | 0 | 351.3 |

Experiments were conducted in 50 mL round bottom flasks using 10 mL of the fermentation broth supernatant. pH values were set by 1.0 M and 0.1 M NaOH solution. Progress of reaction and results were measured by HPLC. The HPLC samples were prepared in the extraction buffer (0.1 M phosphate buffer with 1% (w/v) ascorbic acid). All reactions were stirred protected from the light for 48 hours at ambient temperature (25° C.).

Required pH values were adjusted with 1 M and 0.1 M HCl or NaOH. Reactions at lower pH values are slower and maintain relatively high sum of folates (Table 10, entries 2-4). On the contrary, reactions at higher pH values (Table 10, entries 5-7) improve the consumption of 10-formyldihydrofolic acid albeit significantly reduce the sum of the folates. We anticipate we could use alternative reagents for oxidation such as hydrogen peroxide or sodium periodate.

Representative Experimental Procedure:

Fermentation broth was centrifuged at 4,500 rpm and the supernatant decanted. The 10 mL of fermentation broth supernatant was pipetted into the 50 mL round bottom flasks equipped with stirring bars, pH meter and aluminum foil for light protection. Sodium hydroxide or hydrochloric acid (1.0 M and 0.1 M for fine tuning) was added dropwise to set the pH value and reaction was stirred vigorously for 24 hours under the ambient temperature (25° C.). The reaction mixture was purged with an air from the balloon. After 48 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4,500 rpm, filtered through 0.22 μm filter and analyzed on HPLC.

TABLE 11
Effect of hydrogen peroxide concentration on oxidation
of 10-formyldihydrofolic acid to
10-formylfolic acid in the fermentation broth supernatant

| | | | | | | | | SUM |
|---|---|---|---|---|---|---|---|---|
| | | | | | 10F-DHF | 10F-FA | FA | FOL |
| exp | oxidant | mg/L | time | temp | mg/L | mg/L | mg/L | mg/L |
| 1 | — | 0 | 0 hr | 25° C. | 735.5 | 137.4 | 0 | 872.9 |
| 2 | H₂O₂ | 50 | 24 hr | 25° C. | 390.8 | 299.5 | 12 | 702.4 |
| 3 | H₂O₂ | 100 | 24 hr | 25° C. | 397.3 | 308.7 | 24.2 | 730.2 |
| 4 | H₂O₂ | 250 | 24 hr | 25° C. | 355 | 325.4 | 12.7 | 693.1 |
| 5 | H₂O₂ | 500 | 24 hr | 25° C. | 383.5 | 315.5 | 12.9 | 711.9 |
| 6 | H₂O₂ | 50 | 48 hr | 25° C. | 193.5 | 354.7 | 0 | 548.1 |
| 7 | H₂O₂ | 100 | 48 hr | 25° C. | 183.4 | 529.5 | 0 | 712.8 |
| 8 | H₂O₂ | 250 | 48 hr | 25° C. | 185.9 | 534.3 | 0 | 720.1 |
| 9 | H₂O₂ | 500 | 48 hr | 25° C. | 174.4 | 539.6 | 0 | 714.0 |

Experiments were conducted in 50 mL round bottom flasks using 10 mL of the fermentation broth supernatant. Hydrogen peroxide was added dropwise as 30% solution in water. Progress of reaction and results were measured by HPLC. The HPLC samples were prepared in the extraction buffer (0.1 M phosphate buffer with 1% (w/v) ascorbic acid). All reactions were stirred protected from the light for 48 hours at ambient temperature (25° C.).

Hydrogen peroxide, an alternative oxidant for the oxidative conversion of 10-formyldihydrofolic acid to 10-formylfolic acid was added in concentration range from 50-500 mg/L thus providing more advanced results (Table 11). During the first 24 hours of reaction, the concentration of 10-formyldihydrofolic acid dropped to 50% of its initial value. Prolongation of reaction to 48 hours provided a good conversion thus maintaining a relatively high sum of total folates.

Representative Experimental Procedure:

Fermentation broth was centrifuged at 4,500 rpm and the supernatant decanted. The 10 mL of fermentation broth supernatant was pipetted into the 50 mL round bottom flasks equipped with stirring bars, pH meter and aluminum foil for light protection. Hydrogen peroxide was added dropwise as 30% solution in water and the reaction mixture stirred vigorously for 24-48 hours under the ambient temperature (25° C.). After 48 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4,500 rpm, filtered through 0.22 μm filter and analyzed on HPLC.

TABLE 12
Effect of sodium periodate concentration on oxidation of
10-formyldihydrofolic acid to 10-formylfolic acid
in the fermentation broth supernatant

| | | | | | | | | SUM |
|---|---|---|---|---|---|---|---|---|
| | | | | | 10F-DHF | 10F-FA | FA | FOL |
| exp | oxidant | mg/L | time | temp | mg/L | mg/L | mg/L | mg/L |
| 1 | — | 0 | 0 hr | 25° C. | 735.5 | 137.4 | 0 | 872.9 |
| 2 | NaIO₄ | 5 | 24 hr | 25° C. | 278.5 | 326.5 | 34.1 | 639.1 |
| 3 | NaIO₄ | 5 | 48 hr | 25° C. | 111.8 | 376.7 | 40.3 | 528.8 |
| 4 | NaIO₄ | 10 | 24 hr | 25° C. | 84.6 | 449.6 | 212.6 | 746.8 |
| 5 | NaIO₄ | 10 | 48 hr | 25° C. | 0 | 575.6 | 251.1 | 826.7 |

Experiments were conducted in 50 mL round bottom flasks using 10 mL of the fermentation broth supernatant. Sodium periodate was added in a single portion. Progress of reaction and results were measured by HPLC. The HPLC samples were prepared in the extraction buffer (0.1 M phosphate buffer with 1% (w/v) ascorbic acid). All reactions were stirred protected from the light for 48 hours at ambient temperature (25° C.).

Sodium periodate is often used as the reagent of choice for capricious substrates. Our initial experimentation with this reagent revealed that the effective concentration for the oxidative conversion is between 1-10 g/L. Sodium periodate was added in two different concentrations, 5 g/L and 10 g/L. During the first 24 hours of reaction, the concertation of 10-formyldihydrofolic acid dropped significantly from its initial value (Table 12). Prolongation of reaction to 48 hours provided an excellent conversion thus maintaining a relatively high sum of total folates.

Representative Experimental Procedure:

Fermentation broth was centrifuged at 4,500 rpm and the supernatant decanted. The 10 mL of fermentation broth supernatant was pipetted into the 50 mL round bottom flasks equipped with stirring bars, pH meter and aluminum foil for light protection. Sodium periodate was added in a single portion and the reaction mixture stirred vigorously for 24 hours under the ambient temperature (25° C.). After 48 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4,500 rpm, filtered through 0.22 μm filter and analyzed on HPLC.

### Example 18: Production of Folates in 5 L Bioreactor Volume

The production of folates can be greatly improved in bioreactors where appropriate conditions are used for the cultivation and production of folates. The process includes the preparation of the pre-culture and the main fed-batch bioprocess.

i) Preparation of the Pre-Culture

The pre-culture medium (FOL-MC, Table 13) in flasks is seeded with the working cell bank of strain FL179 and cultivated on a rotary shaker at 37° C. and 220 RPM (2″ throw) for 11-14 hours.

ii) Fed-Batch Bioprocess

The production of folates is carried out in a 5 L bioreactor using the FOL-ME medium (Table 14). The bioreactor starting parameters are Agitation=600 RPM, Aeration=1 vvm, pH is controlled at 7 using ammonium hydroxide solution. The bioreactor is inoculated with 10% of the pre-culture. The DO is controlled by agitation and airflow to keep the air saturation above 30%. When glucose in the fermentation broth is depleted, feeding of a glucose and CSL mixture (Table 15) is started. The rate of feed addition needs to be carefully controlled and the feeding rate is controlled at a level, which does not lead to acetoin (not more than 10 g/L) accumulation. If no acetoin is detected in the fermentation broth the feeding rate is too low. para-aminobenzoic acid (PABA) concentration in the fermentation broth needs to be measured at regular intervals and kept above 500 mg/L by batch feeding of a concentrated PABA stock solution (50 g/L). The bioprocess is usually finished in 50 hours. Folates production bioprocess profile is shown in FIG. 17.

TABLE 13
FOL-MC pre-culture medium

| Component | Amount |
|---|---|
| Molasses | 20 g/L |
| Corn steep liquor (CSL) | 20 g/L |
| Yeast | 5 g/L |
| (NH4)2SO4 | 5 g/L |
| MgSO4 × 7H2O | 0.5 g/L |
| KH2PO4 | 1.5 g/L |
| K2HPO4 | 3.5 g/L |
| glucose | 7.5 g/L |
| Kanamycin | 10 mg/L |
| Tetracycline | 10 mg/L |

TABLE 14
FOL-ME production medium

| Component | Amount |
|---|---|
| Soybean powder | 25 g/L |
| Corn steep liquor (CSL) | 40 g/L |
| Yeast | 5 g/L |
| (NH4)2SO4 | 4 g/L |
| MgSO4 × 7H2O | 2.05 g/L |
| KH2PO4 | 1.5 g/L |
| K2HPO4 | 3.5 g/L |
| Glucose | 30 g/L |
| Kanamycin | 10 mg/L |
| Tetracycline | 10 mg/L |
| Sodium para- | 1 g/L |
| aminobenzoate | |
| (PABA) | |

TABLE 15
Feeding solution (glucose + CSL)

| Component | Amount |
|---|---|
| Glucose monohydrate | 400 g/L |
| Corn steep liquor (CSL) | 310 g/L |

### Example 19: Determination of Expression Levels of Folate Biosynthetic Genes Using qPCR

Culture growth conditions: B. subtilis culture was grown in LB medium to the exponential phase. The culture was mixed with 2 volumes of the RNA protect Bacteria Reagent (QIAGEN), centrifuged for 10 min at 4500 rpm and frozen at −80° C. or processed immediately. Cell pellet was resuspended in 200 μL of TE buffer containing 1 mg/mL lysozyme for 15 min in order to remove the cell wall. RNA was isolated by using QIAGEN Rneasy mini kit according to the manufacturer protocol. The obtained RNA was checked for concentration and quality spectrophotometrically. The isolated RNA was treated with DNase (Ambion kit) and reverse-transcribed to cDNA by using RevertAid H Minus First Strand cDNA Synthesis Kit (Thermo Scientific). The obtained cDNA was diluted and the final yield of cDNA is cca 2.5 ng/μL.

The obtained cDNA was analysed by qPCR analysis (StepOne Real-Time PCR System, Applied Biosystems) with SYBR Green I (Thermo Scientific) detection. The expression of the folate operon genes in the integrated B. subtilis artificial folate operon genes folP, folK, folE, dfrA was quantified by real time quantitative PCR (qPCR) technique.

Internal control gene used as reference for normalization of quantitative qPCR expression data, 16S rRNA gene from B. subtilis was used. The expression of the folate biosynthesis genes was determined using specific set of primers (primer pair SEQ ID NO:59 and SEQ ID NO:60 for folP gene, primer pair SEQ ID NO:61 and SEQ ID NO:62 for folK gene, primer pair SEQ ID NO:63 and SEQ ID NO:64 for folE gene, primer pair SEQ ID NO:65 and SEQ ID NO:66 for dfrA gene) and for 16S gene selected as internal control primer pair SEQ ID NO:69 and SEQ ID NO:70 were used. The qPCR analysis was run on StepOne™ Real-Time PCR System and quantification was performed by using the 2^{-ΔΔCT} method.

The best folate producing strain FL722 bearing multicopy of synthetic folate operons at two separate genome locations (amyE and lacA) was confirmed to have the strongest expression levels of folate biosynthetic genes.

### Example 20: Chemical Conversion of 10-Formyl Folic Acid to Folic Acid

Acid-Mediated Deformylation

Deformylation of 10-formylfolic acid was conducted on 0.01 mmol scale (5 mg). 10-formylfolic acid was weighed in the 2 mL Eppendorf tube equipped with a stirring bar and suspended in distilled water (1 mL). The suspension was treated with acid (50 equiv., 0.5 mmol) and allowed to stir for 16 hours at ambient temperature. Subsequently, a suspension (200 μL) was diluted with DMSO (800 μL), homogenized on the vortex stirrer and analyzed on HPLC. Results of deformylation are presented in Table 16.

TABLE 16
Effect of different acids on of N-deformylation of 10-formylfolic acid

| | | | | | | | | conv. |
|---|---|---|---|---|---|---|---|---|
| exp | solvent | acid | eq. | mmol | time | temp | mmol | to FA^{a} |
| 1 | H₂O | HCl | 50 | 0.5 | 16 hr | 25° C. | 0.01 | 98.8% |
| 2 | H₂O | DOWEX | 50 | 0.5 | 16 hr | 25° C. | 0.01 | n.d.^{b} |
| 3 | H₂O | TFA^{c} | 50 | 0.5 | 16 hr | 25° C. | 0.01 | 92.9% |
| 4 | H₂O | TCA^{d} | 50 | 0.5 | 16 hr | 25° C. | 0.01 | 95.3% |
| 5 | H₂O | HCOOH | 50 | 0.5 | 16 hr | 25° C. | 0.01 | 1.1% |
| 6 | H₂O | PTSA^{e} | 50 | 0.5 | 16 hr | 25° C. | 0.01 | 97.8% |
| 7 | H₂O | CH₃COOH | 50 | 0.5 | 16 hr | 25° C. | 0.01 | 0.7% |
| 8 | H₂O | H₂SO₄ | 50 | 0.5 | 16 hr | 25° C. | 0.01 | 100% |

All experiments were conducted in 2 mL Eppendorf tubes using 10-formylfolic acid (5 mg, 0.01 mmol).
^{a}Conversion was measured by HPLC.
^{b}n.d.—not detected. Neither 10-formylfolic acid nor folic acid were detected in this experiment due to a probable adsorption of the analyte to Dowex 50WX2 resin.
^{c}TFA—Trifluoroacetic acid.
^{d}TCA—Trichloroacetic acid.
^{e}PTSA—p-Toluenesulfonic acid.

Deformylation of 10-formylfolic acid with strong inorganic acids proceeded almost quantitatively to folic acid (Table 16, entries 1 and 8). Alternatively, deformylation with stronger organic acids provided folic acid with nearly equal efficiency (Table 16, entries 3, 4 and 6). As expected, deformylation with formic and acetic acid provided no conversion (Table 16, entries 5 and 7). HPLC analysis of deformylation using Dowex 50WX2 resin provided no detection for a starting material nor product since analyte probably remained adsorbed to the resin and requires elution.

Acid-Mediated N-Deformylation of 10-Formylfolic Acid in the Fermentation Broth

In previous experiments we have illustrated that deformylation of 10-formylfolic acid standard using a strong acid provided a clean conversion to folic acid shown in FIG. 15. Herein we applied the same principle on a more complex system, a fermentation broth. To continue experimenting on biological samples, we have selected a hydrochloric acid (HCl) as a deformylation reagent since it is highly effective and less expensive than other acids we studied. HPLC analysis of fermentation broth from Example 18 showed a substantial amount of 10-formylfolic acid among other folates formed during a biosynthesis (10-formylfolic acid 46% Area; 5-imidomethyltetrahydrofolic acid 47% Area and 5-methyltetrahydrofolic acid 7% Area). Samples of fermentation broth were treated with 1 M HCl up to different pH levels (pH=4, 3, 2, 1 and 0) and stirred for 24 hours at ambient temperature (25° C.) protected from light. According to our HPLC assay, only at lower pH levels (pH=1 and 0) deformylation provided a modest amount of folic acid. Based on these results, we are confident that acid-mediated deformylation strategy is potentially applicable during downstream processing of folic acid. In order to develop a cost-effective deformylation protocol of formyl folate species in a complex system such as fermentation broth, further optimization of acid amount and reaction temperature is essential.

Well-stirred fermentation broth from Example 18 was pipetted into six 100 mL round bottom flasks equipped with stirring bars and pH electrode. Hydrochloric acid was added dropwise with stirring to reach several pH values (pH=4, 3, 2, 1, 0) as described in the Table 17.

TABLE 17
Acid-mediated deformylation of the fermentation broth 3101

| exp | V_{FB} | V_{HCl} | V_{Total} | pH |
|---|---|---|---|---|
| 1 | 50 mL | 0.0 mL | 50 mL | 7.0 |
| 2 | 50 mL | 10.2 mL | 60.2 mL | 4.0 |
| 3 | 50 mL | 15.6 mL | 65.6 mL | 3.0 |
| 4 | 50 mL | 21.4 mL | 71.4 mL | 2.0 |
| 5 | 50 mL | 35.3 mL | 85.3 mL | 1.0 |
| 6 | 50 mL | 59.0 mL | 109.3 mL | 0.0 |

Fermentation mixtures were stirred for 24 hours at ambient temperature (25° C.) shielded from the UV light by a wrapping the flasks in the aluminum foil. A controlled sample was prepared under the exact conditions albeit with the absence of acid (experiment 1). After 24 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4500 rpm, filtered through 0.22 μm filter and analyzed on HPLC. The HPLC results were summarized in the Table 18. According to our HPLC assay, only at lower pH levels (pH=1 and 0) deformylation provided a modest amount of folic acid. In conclusion, we have developed an acid-mediated deformylation of 10-formylfolic acid, a major product of fermentation.

TABLE 18
HPLC-based results of acid-mediated deformylation
on the fermentation broth from Example 18

| | | 5-FTHF | 10-FFA | F SUM | FA |
|---|---|---|---|---|---|
| exp | pH | mg/L | mg/L | mg/L | mg/L |
| 1 | 7.0 | 432 | 487 | 919 | 0 |
| 2 | 4.0 | 171 | 567 | 738 | 0 |
| 3 | 3.0 | 97 | 632 | 729 | 0 |
| 4 | 2.0 | 76 | 529 | 605 | 0 |
| 5 | 1.0 | 54 | 326 | 549 | 169 |
| 6 | 0.0 | 37 | 116 | 402 | 249 |

Base-Mediated Deformylation

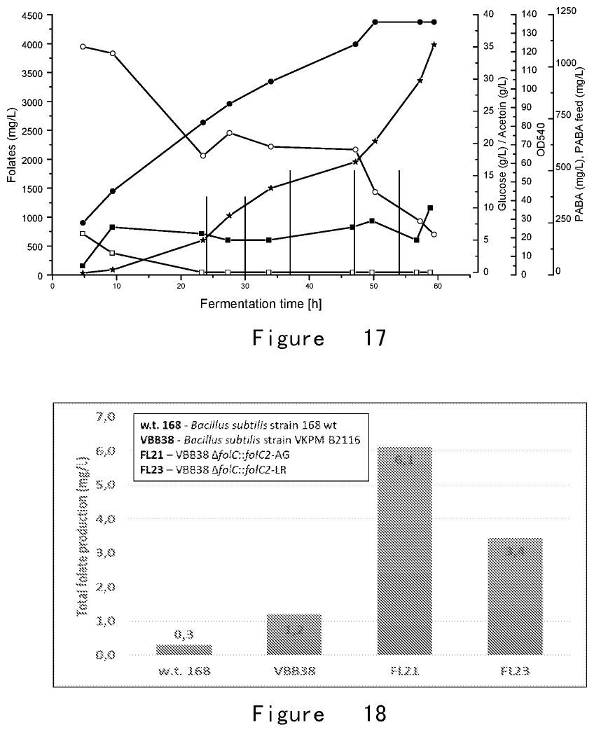

Browsing through the chemical literature, we identified a few reports describing that folic acid displays a greater stability at higher pH values. At such pH values, folic acid exhibit higher solubility which simplifies the synthetic manipulation, purification and downstream processing. Hence, in a series of N-deformylation experiments using 0.1 M NaOH, we are aiming toward clean and efficient conversion from 10-formyl folic acid to folic acid (see FIG. 16) which will simplify the isolation of target product from the fermentation broth. Initial deformylation experiments were carried out on the analytical standard of 10-formylfolic acid using 0.01 mmol scale (5 mg).

Representative Experimental Procedure:

10-formylfolic acid was weighed in the 10 mL round bottom flask equipped with a stirring bar and a rubber septum. The suspension was treated with 0.1 M sodium hydroxide (50 equiv., 0.5 mmol, 5 mL) and allowed to stir for 24-48 hours at ambient temperature protected from light. Subsequently, a solution (100 μL) was diluted with folic acid extraction buffer (900 μL), homogenized on the vortex stirrer and analyzed on HPLC. Three time-dependent aliquots were sampled analyzed on HPLC. Results of deformylation are presented in Table 19. Deformylation of 10-formylfolic acid with 0.1 M NaOH proceeded nearly quantitatively to folic acid during the first sampling after 24 hours (Table 19, entry 1). After stirring for 48 hours, the reaction proceeded to completion according to HPLC analysis. Prolonged stirring under the same conditions disclosed that newly formed folic acid did not undergo to decomposition even after 144 hours (6 days).

TABLE 19
Time scale of N-deformylation of 10-formylfolic acid to folic
acid in the presence 0.1M NaOH

| | | | | 10-FFA | | FA | |
|---|---|---|---|---|---|---|---|
| exp | reagent | time | temp | area | mg/L | area | mg/L |
| 1 | NaOH 0.1M | 24 hr | 25° C. | 16833 | 26 | 1960819 | 1167 |
| | NaOH 0.1M | 48 hr | 25° C. | 0 | 0 | 2095549 | 1247 |
| | NaOH 0.1M | 144 hr | 25° C. | 0 | 0 | 2062398 | 1228 |

Experiments were conducted in 10 mL round bottom flasks using 10-formylfolic acid (5 mg, 0.01 mmol). NaOH 0.1 M was added in excess, 50.0 equivalents, 5 mL. Mass concertation of 10-FFA at the beginning of the experiment is approximately 1000 mg/L. Progress of reaction was measured by HPLC. The HPLC samples were prepared in the extraction buffer (0.1 M phosphate buffer with 1% (w/v) ascorbic acid).

Base-Mediated N-Deformylation of 10-Formylfolic Acid in the Fermentation Broth

In previous experiments we have illustrated that deformylation of 10-formylfolic acid standard using 0.1 M NaOH provided a clean conversion to folic acid shown in FIG. 16. Herein we applied the same principle on a more complex system, a fermentation broth. HPLC analysis of fermentation broth from Example 18 before deformylation showed a substantial amount of 10-formyldihydrofolic acid (10F-DHF; 60% Area); and 10-formylfolic acid (10F-FA; 40% Area). Samples of fermentation broth from Example 18 (10 mL) were treated with different v/v ratios of 0.1 M NaOH (1:1, 1:2, 1:3 and 1:4) and stirred for 24 hours at ambient temperature (25° C.) protected from light. According to our HPLC assay, experiments with fermentation broth/NaOH v/v 1:1 and 1:2 did not lead to deformylation but to oxidative conversion of 10-formyldihydrofolic acid to of 10-formylfolic acid as displayed in Table 20 (entries 2 and 3). Subsequently, when the amount of NaOH was increased in respect to fermentation broth (1:3 and 1:4) a significant amount of folic acid was detected by HPLC as displayed in Table 20 (entries 4 and 5). Interestingly, higher amounts of NaOH somewhat hampered the oxidative conversion of 10F-DHF to 10F-FA since a substantial amount of 10F-DHF was detected by HPLC.

Representative Experimental Procedure:

Well-stirred fermentation broth from Example 18 (10 mL) was pipetted into the 50-100 mL round bottom flasks equipped with stirring bars and aluminum foil for light protection. Sodium hydroxide (0.1 M) was added dropwise and reaction was stirred vigorously for 24 hours under the ambient temperature (25° C.). After 24 hours of stirring, 1 mL of each fermentation broth was diluted in duplicates with 9 mL of extraction buffer (0.1 M phosphate buffer with 1% (w/v) ascorbic acid). The suspensions were stirred on vortex, centrifuged at 4500 rpm, filtered through 0.22 μm filter and analyzed on HPLC.

TABLE 20
Effect of addition of different amounts of NaOH on of
N-deformylation of 10-formylfolic acid in fermentation broth

| | | | | | | | SUM |
|---|---|---|---|---|---|---|---|
| | | | | 10F-DHF | 10F-FA | FA | FOL |
| exp | sample | time | temp | mg/L | mg/L | mg/L | mg/L |
| 1 | FB3148 | 0 hr | 25° C. | 455 | 302 | 0 | 757 |
| 2 | FB3148/NaOH | 24 hr | 25° C. | 87 | 428 | 0 | 515 |
| | 0.1M (1:1 v/v) | | | | | | |
| 3 | FB3148/NaOH | 24 hr | 25° C. | 0 | 623 | 0 | 623 |
| | 0.1M (1:2 v/v) | | | | | | |
| 4 | FB3148/NaOH | 24 hr | 25° C. | 47 | 451 | 302 | 800 |
| | 0.1M (1:3 v/v) | | | | | | |
| 5 | FB3148/NaOH | 24 hr | 25° C. | 350 | 284 | 222 | 856 |
| | 0.1M (1:4 v/v) | | | | | | |

Experiments were conducted in 50-100 mL round bottom flasks using the fermentation broth from Example 18 (FB3148, 10 mL). NaOH 0.1 M was added based on the volume/volume ratio in respect to FB3148 (1:1, 1:2, 1:3 and 1:4). Progress of reaction and results were measured by HPLC. The HPLC samples were prepared in the extraction buffer (0.1 M phosphate buffer with 1% (w/v) ascorbic acid). All reactions were stirred protected from the light for 24 hours at ambient temperature (25° C.).

### Example 21: Isolation of 10-Formyl Folic Acid

After harvesting, a fermentation broth containing 50 g of folic acid was adjusted to pH=12 using 5M aqueous NaOH. The solution was centrifuged at 10000 rpm for 15 minutes at 4 ̆C. To a supernatant, 50 g of calcium hydroxide was added and suspension was stirred at room temperature for 2 hours. The resulting suspension was allowed to settle, decanted and the supernatant liquid was filtered with the aid of 100 of diatomaceous earth (Celite). The filter cake was washed with 500 mL of water and filtered. The filtrates were combined and diluted to a final volume of 10 liters. The dilute alkaline solution of clarified folic acid was adjusted to a pH 7.0 with 1N HCl, heated to 70° C. and then cooled to a room temperature. Next, the solution was filtered to remove impurities that precipitate at neutral pH. A clarified filtrate was adjusted to pH=3 using 1N HCl and cooled on ice for 4 hours. The suspension was filtered off and redissolved in 8 L of hot alkaline solution with pH=12 (adjusted with 1M NaOH). To this solution, 50 grams of activated charcoal (1 equivalent/weight of folic acid) was added and the solution was heated to 50° C. and stirred for 30 minutes. The suspension was filtered, the filter cake was washed with 3 L of alkalinized aqueous solution (pH=12 adjusted with NaOH). Filtrates were combined and pH was adjusted to 3.0 utilizing 1N HCl, added during continuous stirring. The resulting slurry was cooled on ice for 24 h or overnight. The suspension was filtered off and resuspended in 1 L of acidified aqueous solution having a pH=3 (pH was adjusted with 1N HCl). The suspension was again filtered and the resulting filter cake was then frozen and dried to obtain 43 grams of folic acid, which contained 10% of moisture and assayed 90.1% folic acid on an anhydrous basis.

### Example 22 Isolation of Folic Acid

After harvesting, a fermentation broth containing 30 g of folic acid was adjusted to pH=10 using 1M aqueous NaOH. The solution was centrifuged at 10000 rpm for 15 minutes at 4 ̆C. The resulting supernatant was adjusted to a pH 4.0 with 1N HCl, heated to 70° C. and then cooled to a room temperature. Next, the solution was filtered with the aid of 100 g of Celite. Filter cake was resuspended in 5 L of alkaline solution with pH=10 (adjusted with 1M NaOH). To this solution, 50 grams of activated charcoal (1 equivalent/weight of folic acid) was added and the solution was heated to 50° C. and stirred for 30 minutes. The suspension was filtered, the filter cake was washed with 2 L of alkalinized aqueous solution (pH=12 adjusted with NaOH). Filtrates were combined and pH was adjusted to 3.0 utilizing 1N HCl, added during continuous stirring. The resulting precipitate was cooled on ice for 16-24 h or then filtered off and resuspended in 1 L of acidified aqueous solution having a pH=3 (pH was adjusted with 1N HCl). The suspension was again filtered and the resulting precipitate cake was dried to obtain 21 grams of 10-formyl folic acid, which was assayed 92%.

### Comparative Example 1

Total folate production was determined for B. subtilis wild type strain “168”, our starting non-GMO strain VBB38 (strain VKPM B2116=B. subtilis VNII Genetika 304) and its transformants in which native folC gene was replaced in one step by a heterologous folC2 (FOL3) gene from either A. gossypii (B. subtilis strain FL21) or L. reuteri (B. subtilis strain FL23). Strains were tested at the shaker scale (5 ml production medium MD) and total folates were determent by using standard microbiological assay for folate detection.

The result was shown that knockout mutants of deletion of B. subtilis native folC gene alone without simultaneous heterologous folC2 gene expression were not able to grow in standard cultivation conditions (T=37 C, aerobically in nutrient rich LB medium).

### LITERATURES

1. Hjortmo S, Patring J, Andlid T. 2008 Growth rate and medium composition strongly affect folate content in Saccharomyces cerevisiae. Int J Food Microbiol. 123(1-2):93-100.
2. McGuire J J and Bertino J R. 1981. Enzymatic synthesis and function of folylpolyglutamates. Mol Cell Biochem 38 Spec No (Pt 1):19-48.
3. Reed, L S, Archer M C. 1980. Oxidation of tetrahydrofolic acid by air. J Agric Food Chem. 28(4):801-805.
4. Rossi, M., Raimondi, S., Costantino, L., Amaretti, A., 2016. Folate: Relevance of Chemical and Microbial Production. Industrial Biotechnology of Vitamins, Biopigments, and Antioxidants. Wiley-VCH Verlag GmbH & Co. KGaA, Weinheim, Germany, pp. 103-128.
5. Scaglione and Panzavolta. 2014. Folate, folic acid and 5-methyltetrahydrofolate are not the same thing. Xenobiotica. 44(5):480-488.
6. Serrano-Amatriain C, Ledesma-Amaro R, López-Nicolás R, Ros G, Jiménez A, Revuelta J L. 2016. Folic acid production by engineered Ashbya gossypii. Metab Eng. 38:473-482.
7. Sybesma W, Starrenburg M, Kleerebezem M, Mierau I, de Vos W M, Hugenholtz J. 2003a. Increased production of folate by metabolic engineering of Lactococcus lactis. Appl Environ Microbiol. 69(6):3069-3076.
8. Sybesma, W., Starrenburg, M., Tijsseling, L., Hoefnagel, M. H. N., Hugenholtz, J., 2003b. Effects of cultivation conditions on folate production by lactic acid bacteria. Applied and Environmental Microbiology. 69(8):4542-4548.
9. Sybesma W, Van Den Born E, Starrenburg M, Mierau I, Kleerebezem M, De Vos W M, Hugenholtz J. 2003c. Controlled modulation of folate polyglutamyl tail length by metabolic engineering of Lactococcus lactis. Appl Environ Microbiol. 69(12):7101-7107.
10. Zeigler D R, Prágai Z, Rodriguez S, Chevreux B, Muffler A, Albert T, Bai R, Wyss M, Perkins J B. 2008. The origins of 168, W23 and other Bacillus subtilis legacy strains, Journal of Bacteriology. 190(21):6983-6995
11. Zhu T, Pan Z, Domagalski N, Koepsel R, Ataai M M, Domach M M. 2005. Engineering of Bacillus subtilis for enhanced total synthesis of folic acid. Appl Environ Microbiol. 71(11):7122-7129.12. Walkey C J, Kitts D D, Liu Y, van Vuuren H J J. 2015. Bioengineering yeast to enhance folate levels in wine. Process Biochem 50(2):205-210.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A genetically engineered strain for the synthesis of a folate, a salt thereof, a precursor thereof, or an intermediate thereof, wherein the expression level of the endogenous folC gene in the engineered strain is decreased, and an exogenous folC gene is introduced and the engineered strain has a significantly improved production capacity of a folate, a precursor, or an intermediate thereof compared to its starting strain.

2. The genetically engineered strain of claim 1, wherein the structural formula of a folate, a salt, a precursor, or an intermediate thereof is as shown in Formula I: 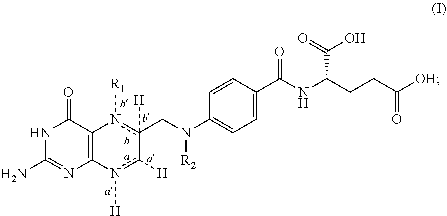
wherein, when a is single bond, a′ is none or when a′ is a single bond, a is none;
when b is a single bond, b′ is none or when b′ is a single bond, b is none;
R1 is selected from the group consisting of: —H, —CH₃ (5-methyl), —CHO (5-formyl), —CH═ or ═CH— (5,10-methenyl), —CH₂— (5,10-methylene), —CH═NH (5-formimino-) and a combination thereof;
R2 is selected from the group consisting of: —H. —CHO (10-formyl), —CH═, ═CH— (5,10-methenyl), —CH₂— (5,10-methylene) and a combination thereof.

3. The genetically engineered strain of claim 1, wherein the starting strain of the engineered strain is selected from the group consisting of Lactococcus lactis, Bacillus subtilis, Ashbya gossypii and a combination thereof.

4. The genetically engineered strain of claim 1, wherein the exogenous folC gene is derived from Ashbya gossypii, or Lactobacillus reuteri.

5. The genetically engineered strain of claim 1, wherein the expression product of the exogenous folC gene comprises a polypeptide or a derivative polypeptide thereof selected from the group consisting of: dihydrofolate synthase (DHFS-EC 6.3.2.12).

6. The genetically engineered strain of claim 5, wherein the amino acid sequence of the dihydrofolate synthase is as shown in SEQ ID NO.: 22 or 23.

7. The genetically engineered strain of claim 1, wherein a gene encoding a folate biosynthetic enzyme is introduced or up-regulated in the engineered strain.

8. The genetically engineered strain of claim 7, wherein the folate biosynthetic gene is selected from the group consisting of folE/mtrA, folB, folK, folP/sul, folA/dfrA, and a combination thereof.

9. The genetically engineered strain of claim 7, wherein the folate biosynthetic gene is derived from a bacterium, preferably from a bacterium of the Bacillus species, most preferably from Bacillus subtilis or Lactococcus lactis or Ashbya gossypii.

10. A method for preparing a folate, a salt thereof, a precursor thereof, or an intermediate thereof, comprising the steps of:
(i) providing the engineered strain of claim 1;
(ii) cultivating the engineered strain described in the step (i), thereby obtaining a fermentation product containing one or more compounds of the folate, the salt thereof, the precursor thereof, or the intermediate thereof;
(iii) Optionally, the fermentation product obtained in the step (ii) is subjected to separation and purification to further obtain one or more compounds of the folate, the salt thereof, the precursor thereof, or the intermediate thereof;
(iv) Optionally, the product obtained in the steps (ii) or (iii) is subjected to acidic or alkaline conditions to further obtain a different compound of the folate, the salt thereof, the precursor thereof, or the intermediate thereof;
wherein the structural formula of a folate, a salt, a precursor, or an intermediate thereof is as shown in Formula I: 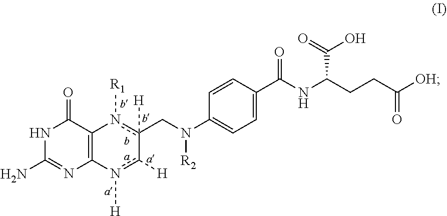
and R₁, R₂, a, a′, b, b′ are defined as above.

11. The method of claim 10, wherein the folate, the salt thereof, the precursor thereof, or the intermediate thereof is folic acid

12. A method for preparing a folate, a precursor, or an intermediate thereof, comprising the steps of:
(i) providing the engineered strain of claim 1;
(ii) cultivating the engineered strain described in the step (i), thereby obtaining a folate-containing fermentation product;
(iii) Optionally, the fermentation product obtained in the step (ii) is subjected to separation and purification to further obtain a folic acid, a precursor, or an intermediate thereof.

13. The method of claim 12, wherein the method further comprises the step of adding para-aminobenzoic acid (PABA) during the cultivation process of step (ii).

14. A method of preparing the engineered strain of claim 1, comprising the steps of:
(a) decreasing the expression level of the endogenous folC gene in the starting strain, and introducing the exogenous folC gene, thereby obtaining the engineered strain of claim 1.

15. The method of claim 14, wherein the method further comprises the step (b) of introducing or upregulating a folate synthesis regulatory gene in the starting strain.

16. Use of an engineered strain according to claim 1, which is used as an engineered strain for fermentative production of a folate, a salt, a precursor or an intermediate thereof.

17. A genetically engineered microorganism, which has been modified to i) have a decreased expression level of the endogenous gene encoding a polypeptide having both dihydrofolate synthase activity and folylpolyglutamate synthetase activity compared to an otherwise identical microorganism (reference microorganism), and ii) express a heterologous polypeptide having only dihydrofolate synthase activity.

18. A method for preparing folate or a salt, precursor or intermediate thereof, comprising i) cultivating a genetically engineered microorganism according to the fifth aspect of the present invention in a culture medium under suitable culture conditions to obtain a fermentation product containing said folic acid, precursor or intermediate thereof; and ii) optionally, separating and/or purifying said folic acid, precursor or intermediate thereof.
